(19) United States
(12) Patent Application Publication
(10) Pub. No.: US 2026/0290423 A1
(43) Pub. Date: Sep. 24, 2026

(54) REFRESH SCHEME WITH SAME TIER ACTIVATION

(71) Applicant: Micron Technology, Inc., Boise, ID (US)

(72) Inventors: Hiromasa Takeda, Tokyo (JP); Kyoichi Nagata, Kanagawa (JP); Hiroki Fujisawa, Sagamihara-shi (JP)

(73) Assignee: Micron Technology, Inc., Boise, ID (US)

(21) Appl. No.: 19/556,170

(22) Filed: Mar. 4, 2026

Related U.S. Application Data
(60) Provisional application No. 63/776,613, filed on Mar. 24, 2025.

(51) Int. Cl. G11C 11/406; G11C 11/408; G11C 11/4091; H10B 12/00

(52) U.S. Cl. CPC G11C 11/40615; G11C 11/4085; G11C 11/4091; H10B 12/50

(57) ABSTRACT

Apparatuses, systems, and methods for a refresh scheme that refreshes word lines in a same tier in 3D-DRAM. The 3D memory device includes multiple memory patches each of which includes a 3D array of memory cells arranged at the intersection of word lines running in a first direction, local digit lines (LDLs) running in a second direction, and global digit lines (GDLs) running in a third directions. The 3D memory array may be refreshed on a word line-by-word line basis as part of a refresh operation. During the refresh operation, one or more word lines may be refreshed in the 3D memory device. The word lines being refreshed may all be at a same tier of the 3D memory device. The tier may represent the depth at which the refreshed word line exists within a stack of word lines in the 3D memory array.

### CROSS REFERENCE TO RELATED APPLICATIONS

This application claims the benefit under 35 U.S.C. § 119 of the earlier filing date of U.S. Provisional Application Serial No. 63/776,613 filed Mar. 24, 2025. The aforementioned application is incorporated herein by reference, in its entirety, for any purpose.

### BACKGROUND

Memory devices, such as DRAM devices have generally decreased in size and increased in capacity over time. Many of these gains have been accomplished by miniaturizing circuits such as the sense amplifier, sub-word line driver, and so for forth across different generations of memory devices. However, it is difficult to decrease the size of components below certain size thresholds, which makes it difficult to continue to improve memory device capacity and size in this manner.

It may be possible to increase memory device size and capacity by arranging memory cells in a three-dimensional grid instead of a two-dimensional array. For example, multiple memory chips may be stacked on top of each other. However, this increases the thickness of the overall memory device to an extent that may be difficult to accommodate and the number of stacked chips is limited by concerns such as the length of signal lines through the stack, alignment of timing signals, and so forth. Other technologies may be used to generate 3D arrays of memory cells, such as the 3D arrays found in flash memory, but these may have limitations in the speed at which the memory device operates and are generally much slower than the speeds required of a DRAM device. There may be a need for 3D memory devices which operate at high speeds.

### BRIEF DESCRIPTION OF DRAWINGS

FIG. 1 is a block diagram of a 3D memory device according to some embodiments of the present disclosure.

FIG. 2A is a perspective drawing of a memory device according to some embodiments of the present disclosure.

FIGS. 2B-2G show example ‘top down’ views of different example layouts of a memory quilt of a memory device according to some example embodiments of the present disclosure.

FIG. 3 is a perspective schematic diagram of a portion of a 3D memory array according to some embodiments of the present disclosure.

FIG. 4 is a perspective view of a portion of a 3D memory device according to some embodiments of the present disclosure.

FIG. 5 is a cross sectional view of a 3D memory device according to some embodiments of the present disclosure.

FIG. 6 is a cross sectional view of a 3D memory device according to some embodiments of the present disclosure.

FIG. 7 is a cross sectional view of a 3D memory device according to some embodiments of the present disclosure.

FIG. 8 is a perspective view of a portion of a memory device according to some embodiments of the present disclosure.

FIG. 9 is a cross sectional view of a memory device according to some embodiments of the present disclosure.

FIG. 10 is a cross sectional view of a memory device according to some embodiments of the present disclosure.

FIG. 11 is a cross sectional view of a memory device according to some embodiments of the present disclosure.

FIG. 12 is a perspective view of a memory quilt of a memory device according to some embodiments of the present disclosure.

FIG. 13 is a top-down view of a memory quilt according to some embodiments of the present disclosure.

FIG. 14 is a cross sectional view of a memory device according to some embodiments of the present disclosure.

FIG. 15 is a cross sectional view of a memory device according to some embodiments of the present disclosure.

FIG. 16 is a perspective view of a memory quilt of a memory device according to some embodiments of the present disclosure.

FIG. 17 is a top-down view of a memory quilt according to some embodiments of the present disclosure.

FIG. 18 is a cross sectional view of a memory device according to some embodiments of the present disclosure.

FIG. 19 is a cross sectional view of a memory device according to some embodiments of the present disclosure.

FIG. 20 is a cross sectional view of a memory device according to some embodiments of the present disclosure.

FIG. 21 is a block diagram of a 3D memory device layout according to some example embodiments of the present disclosure.

FIG. 22 is a perspective block diagram of a portion of a 3D memory device according to some embodiments of the present disclosure.

FIG. 23 is a cross sectional schematic view of a 3D memory device according to some embodiments of the present disclosure.

FIG. 24 is a timing diagram for example refresh operations according to some embodiments of the present disclosure.

FIG. 25 is a flow chart of a refresh operation according to some embodiments of the present disclosure.

FIG. 26 is a flow chart of a refresh operation according to some embodiments of the present disclosure.

### DETAILED DESCRIPTION

The following description of certain embodiments is merely exemplary in nature and is in no way intended to limit the scope of the disclosure or its applications or uses. In the following detailed description of embodiments of the present apparatuses, systems, methods, and combinations thereof, reference is made to the accompanying drawings. The drawings are shown by way of illustration of specific example embodiments of how the described apparatuses, systems, methods, or combinations thereof may be practiced. These embodiments are described in sufficient detail to enable those skilled in the art to practice presently disclosed apparatuses, systems, methods, and combinations thereof, and it is to be understood that other embodiments may be utilized and that structural and logical changes may be made without departing from the spirit and scope of the disclosure. Moreover, for the purpose of clarity, detailed descriptions of certain features will not be discussed when they would be apparent to those with skill in the art so as not to obscure the description of embodiments of the disclosure. The following detailed description is therefore not to be taken in a limiting sense, and the scope of the disclosure is defined only by the appended claims.

A memory device includes a memory array. The memory array includes a number of memory cells which store information. For example each memory cell may store a single bit of information as a charge on a capacitive element. In a conventional 2D memory device, the memory cells are logically organized at the intersection of rows and columns. Conductive elements known as word lines couple memory cells along a row, and conductive elements known as digit lines or bit lines couple memory cells along a column. Accordingly, a row address may be used to specify a word line and a column address may be used to specify one or more of the digit lines.

The memory array may be formed of repeating units, referred to as ‘sections’. These sections include one or more patches of the memory array and various circuits which support the operation of that patch. For example, a section may include the patch, sense amplifiers (SA) coupled to the digit lines of that patch, and sub-word line drivers (SWD) coupled to the word lines of the patch. For example, in a conventional 2D memory device, the memory array patch may be generally rectangular with SAs positioned along a ‘top’ and ‘bottom’ edge of the patch and SWDs positioned along a ‘left’ and ‘right’ edge. The top row of sense amplifiers are coupled to either even or odd digit lines (in this and in an adjacent patch), the bottom row of sense amplifiers are coupled to the other digit lines (in this and in an adjacent patch). The SWDs along the edges are similarly coupled to alternate word lines in the patch.

While this layout may be efficient for 2D memory devices, when a third dimension is added, it may become more complicated to generate sections which are compact and which repeat across the memory device. For example, the need for connections to run in a third axis may complicate the placement of components such as SAs and SWDs compared to 2D memory. However, the expansion into a third dimension may also provide opportunities for new locations to place components, such as above the memory array. In addition, the expansion into a third dimension may increase the memory cell density enough that it is possible to relax the pitch, or spacing, between components, which may allow more flexible layouts to be used compared to the layouts which are possible with the stringent pitch requirements of 2D memory. For example, a 3D memory device may have generally greater spacing between components such as word lines, sense amplifiers, and sub-word line drivers compared to a 2D memory. There may be a need for new layouts in 3D-DRAM devices to take advantage of the flexibility that 3D affords.

The present disclosure is drawn to apparatuses, systems, and methods for the layouts and operations of various components in 3D-DRAM. An example 3D-DRAM device includes memory cells arranged at the intersection of word lines and local digit lines with the local digit lines coupled together by global digit lines. The word lines, local digit lines, and global digit lines may generally extend in directions which are mutually orthogonal to each other. The present application uses the convention that the word lines generally extend along an ‘x’ direction, the local digit lines generally extend along a ‘z’ direction, and the global digit lines generally extend along a ‘y’ direction. The x, y, and z directions may be orthogonal to each other.

In some aspects, the present application relates to the spatial arrangement of various components. As used herein, when a circuit element is referred to as extending in or along a direction or axis, it refers to a component that is primarily extended in that dimension. However, such an element need not be a perfect line and may have portions which do not exclusively run in the given direction. For example, while a word line may primarily extend along the x axis, it may have portions which briefly extend in the y and/or z directions. As used herein, when a component is referred to as being ‘above’ or ‘below’ another component, it means that at least a portion of the footprint of that component overlaps at least a portion of the footprint of the other component when projected on at least one plane but that they are offset in at least one axis. For example, in some embodiments the sense amplifier region may be above the array patch in the z direction. At least a portion of the xy footprint of the sense amplifier region overlaps at least a portion of the xy footprint of the array patch region, however the sense amplifier region occupies a different range of space long the z coordinate than the array patch region.

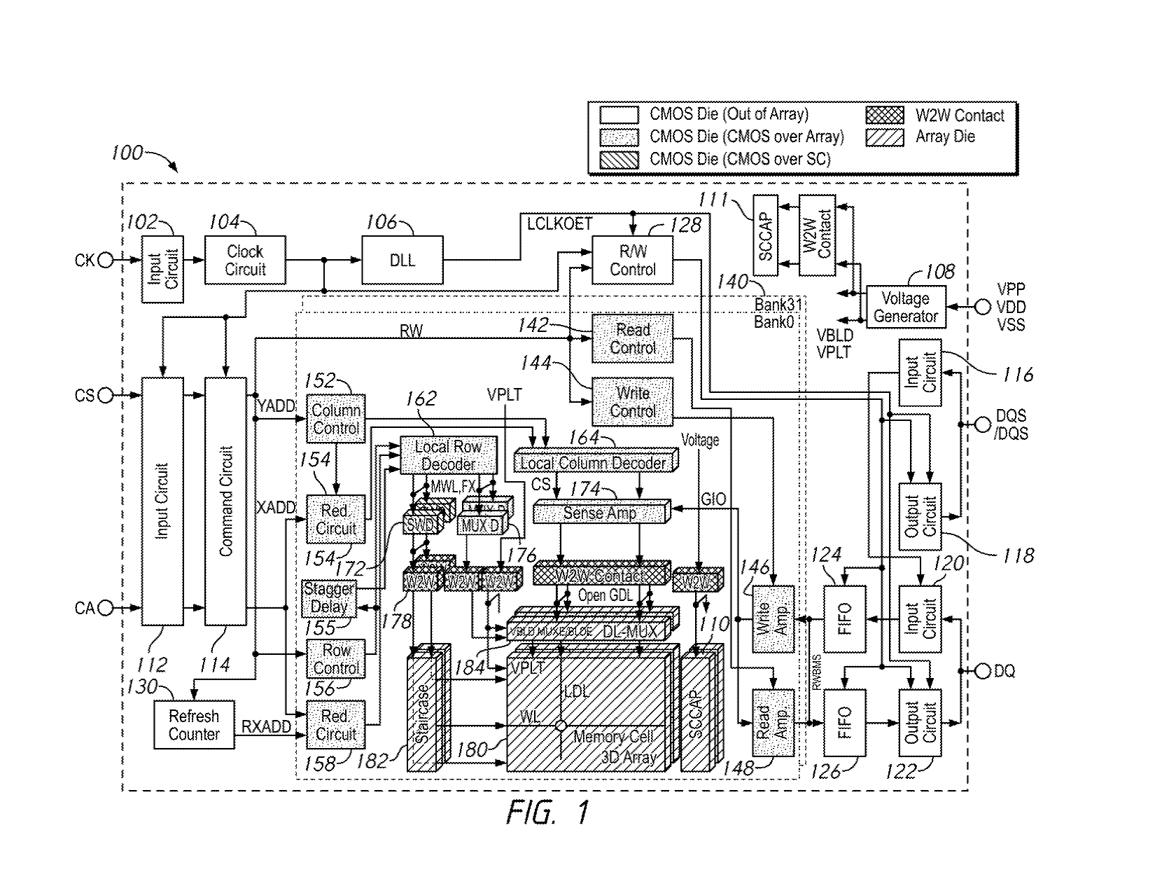

FIG. 1 is a block diagram of a 3D memory device according to some embodiments of the present disclosure. The view of the 3D memory device 100 in FIG. 1 is a block diagram representing the different components of the 3D memory device 100. It does not represent the spatial layout of the components of the device, except where otherwise noted.

The 3D memory device 100 may be coupled to a controller (not shown in FIG. 1) which provides various commands, data, and other signals to the memory device 100 to operate the memory. In some embodiments, the 3D memory device 100 may be a stand-alone device. In some embodiments the 3D memory device 100 may be part of a module that packages together several similar memory devices.

The 3D memory device 100 includes a number of external terminals which receive various signals which are used by the device 100. The signals which are received, as well as the signals within the memory device 100, are generally represented by voltages, which different levels of voltage representing different states of the signal. For example, many of the signals used by the memory device 100 may be binary signals, where a first voltage level represents a logical high and a second voltage level represents a logical low. Example terminals include a clock terminal CK, a chip select terminal CS, a command/address terminal CA, data strobe terminals DQS and /DQS, data terminals DQ, and voltage terminals such as VPP, VDD, and VSS. Any of those example terminals may include one or more individual terminals.

The clock signal CK is received by the clock terminal and provided through an input circuit 102 to a clock circuit 104. The clock signal CK is used to control the timing of operations in the memory device 100. The clock circuit 104 generates one or more internal clock signals based on the external clock signal CK and distributes them to various other components of the memory device 100. The clock circuit 104 also provides the clock signal to a delay locked loop (DLL) circuit 106 which generates a delayed clock signal LCLKOET. The delayed clock circuit may be used to match a timing it takes the clock signal to propagate through the memory device 100 and may be used to time read operations. The 3D memory device 100 also receives and provides data strobes DQS and /DQS through a respective input circuit 116 and output circuit 118. During write operations the memory receives a data strobe signal used to time operations of the input circuit 120 for the data DQ. During read operations the memory 100 provides the delayed clock signal LCLKOET to time the operations of a data output circuit 122 to the data terminal DQ. Buffer circuits 124 and 126 are used to serialize or deserialize data between the memory device and the DQ terminals.

The 3D memory device 100 receives voltages at voltage terminals. The voltages are provided to a voltage generator circuit 108 which generates one or more internal voltages based on the provided voltages. The provided voltages as well as the generated voltages are distributed to the various circuits of the memory device 100. For example, the memory device 100 may receive a ground voltage VSS and a system voltage VDD as well as a voltage VPP. The 3D memory device 100 includes one or more driver circuits which provide voltages to various circuits of the 3D memory device 100. The drivers may provide voltages based on the voltages provided by to the voltage generator 108. For example, the sub-word line drivers 172 provide voltages to drive the word lines in the array 180, the multiplexer drivers 176 provide voltages to driver multiplexer circuits 184, and sense amplifier drivers (not shown) provide voltages to the sense amplifiers 174. The voltages may be driven along conductive elements. In some embodiments, multiple circuits may be coupled in common to a same conductive element providing a voltage, which may be referred to as a ‘rail’.

The 3D memory device 100 includes capacitors 110 and 111 which help regulate voltages in the memory array 180. The capacitors 111 are used to regulate voltages from the voltage generator 108. The capacitors 110 are used to regulate voltages provided to the sense amplifiers. In the example implementation of FIG. 1, the capacitors 110 and 111 represent one or more capacitor regions positioned in the array die. The capacitors 110 may be positioned next to array patches 180. The capacitors 110 may be positioned in a peripheral region. Other arrangements of capacitors may be used in other example embodiments.

The memory receives commands and addresses along a command/address bus coupled to CA terminals. The 3D memory device 100 also receives a chip select signal CS, which is used to time signals along the CA terminals, as well as indicate which memory device 100 is receiving commands and addresses in embodiments where multiple memory devices are packaged together. The CS and CA signals are passed through an input circuit 112 to a command/address circuit 114. Examples of addresses include bank address BADD which specifies a bank of the memory device 100, row address XADD which specifies a row of the device, and column address YADD which specifies a column of the device. Examples of commands include activation commands ACT, pre-charge commands PRE, access commands such as read R or write W, or refresh commands. Certain commands and addresses may generally be received together. For example, the CA terminal may receive a row activation command ACT along with a row address XADD and bank address BADD. Access commands such as read or write may generally be received along with a column address YADD. A read/write control circuit 128 helps manage the RW commands.

The 3D memory device 100 is divided into one or more memory banks 180. In the embodiment of FIG. 1, there are 32 banks, labelled Bank0 to Bank31. More or fewer banks may be used in other example embodiments. Each bank is associated with a bank logic region 140, which includes the memory array 180 of the bank as well as various circuits associated with that bank. These circuits may generally be repeated on a bank-by-bank basis. An example bank logic region 140 may include a column control circuit 152 and column redundancy circuit 154, a row control circuit 156 and row redundancy circuit 158, local row decoder 162, local column decoder 164, sub-word line drivers 172, sense amplifiers 174, multiplexer drivers 176, the array 180 and staircase region 182, digit line multiplexers 184, write amplifiers 146 and read amplifiers 148.

The array 180 is a 3D array with memory cells coupled at the intersection of word lines WL and local digit lines LDL. The view of FIG. 1 shows an example slice of the array through an xz plane, showing an example WL and LDL. The LDLs are coupled together along a y axis by pairs of global digit lines GDL. The pair of GDLs coupled to a same sense amplifier of the sense amplifiers 174. Along the length of the GDL pair, a portion (e.g., a first half) of the LDLs are coupled to one GDL in the pair, and a portion (e.g., a second half) of the LDLs are couple to the other GDL in the pair. In some embodiments, multiplexers 184 are used to select which of the intersecting LDL(s) are coupled to the respective GDL, and through that GDL to the sense amplifier. The local row decoder 162 is used to selectively activate multiplexer drivers 176 based on the row address, which activates respective ones of the multiplexers 184. In some embodiments, the multiplexers 184 and their respective drivers 176 may not be used and may be eliminated.

The row control circuit 156 activates a word line of the memory array 180 based on the row address. The row control circuit 156 provides internal signals to the local row decoder circuits 162. The local row decoder circuit 162 activates a sub-word line driver 172 associated with the word line specified by XADD. The sub-word line driver 172 is coupled to an associated word line WL through the staircase 182. The sub-word line driver selected by the row address activates the associated word line, causing the memory cells along that word line to couple to the intersecting local digit lines LDLs. The LDLs are coupled to one of each of the intersecting pairs of GDLs. The sense amplifier compares the signal along the GDL of the pair coupled to the active word line (through the LDL) to a reference along the GDL of the pair which is not coupled to the active word line. In some embodiments, the local row decoder circuit 162 also activates a multiplexer driver 176 which in turn selects multiplexers 184 to couple the LDLs which intersect the selected word line to the GDLs.

The multiplexers 184 may be an optional component used in some example embodiments. In embodiments where multiplexers are used, the local row decoder circuit 162 receives the row address XADD and uses the row address to determine which multiplexer(s) 176 to activate. Based on the row address the local row decoder circuit 162 provides decoded address to the multiplexer drivers 176, and the multiplexer driver(s) associated with the decoded address provide a multiplexer driver signal. The multiplexers 176 may be grouped together in sets, and the sets are coupled in common to a multiplexer driver 176 provides the multiplexer driver signal, the multiplexers in the set are activated and couple the associated LDL to the respective GDL of the GDL pair. In some embodiments, when the multiplexer driver signal is inactive, the LDL may be decoupled from the GDL and coupled to a ground voltage instead.

A stagger delay circuit 155 controls a timing of the operation of the local row decoder circuit 162. For example, the stagger delay circuit 155 may use internal signals to control when the local row decoder circuit 162 activates the SWD 172 and when it activates the multiplexer driver 176.

The column control circuit 152 selects which global digit lines to couple to global input/output lines GIO based on the column address YADD. During a write operation, the GIO lines are coupled to a write amplifier 146, which provides data received from the input circuit 120 through the GIO lines to the selected GDL lines and through those to the memory cells at the intersection of the selected WL and LDL. During a read operation, the data from the memory cells at the intersection of the selected WL and LDL are coupled through the selected GDL to the read amplifier 148, which provides the read data to the output circuit 122.

The memory bank logic region 140 also includes redundancy circuits 154 and 158. The redundancy circuits 154 and 158 are used as part of repair operations. If a row or column address has been repaired, the redundancy circuits 154 or 158 will direct access to a redundant word line or redundant column.

In an example write operation, data received along the DQ terminals is written to specified memory cells of the 3D memory device 100. The device 100 receives a row activation command along with a row address. The local row decoder 162 selects SWDs 172 to activate the associated word line based on the row address and may activate a multiplexer driver 176. The device 100 receives a write command along with a column address. Data is received by the input circuit 120 and deserialized by a buffer circuit 124 which provides the data to write amplifiers 146. The local column decoder 164 couples selected global digit lines to the GIO lines based on the column address YADD. In some embodiments, a multiplexer driver 176 is activated by the local row decoder 166 and couples a local digit line which intersects the active word line to the respective global digit lines. The data from the write amplifier 146 is written along the GIO lines to GDL line, onto the selected LDL lines and into the memory cells at the intersection of the LDL with the active word line WL.

In an example read operation, data from the array 180 is provided out along the DQ terminals. The device 100 receives a row activation command along with a row address. The local row decoder 162 selects SWDs 172 to activate the associated word line based on the row address. In some embodiments, the global row decoder 166 selects a multiplexer driver 176 to selectively couple one or more selected LDLs to the respective GDL. Data is read out from the memory cells which intersect the active word line through the intersecting LDLs and along the GDLs. The device 100 receives a read command along with a column address. Data is read out from selected ones of the GDLs along the GIO lines to a read amplifier 148. The read amplifier provides the read data to a buffer 126 which serializes the data and provides the serialized data to the output circuit 122.

The 3D memory device 100 may also perform refresh operations. As part of a refresh operation, one or more word lines have the data in the intersecting memory cells refreshed. For example, the memory cells may store information as charge on a capacitive element, and that charge may decay over time. A refresh operation restores that charge to a nominal value. Responsive to a refresh command, a refresh control circuit 130 provides one or more refresh addresses RXADD which specify which word line or word lines should be refreshed.

FIG. 1 generally shows the 3D memory device 100 as a flattened 2D drawing. However certain components may be positioned over other components in an example implementation. For example, the bank logic circuits 140 may be positioned ‘above’ (in the z direction) the memory banks 180. For example, the sub-word line drivers 172 may be positioned above the staircase 182. In some embodiments, the memory device 100 may be printed on two chips, which are then bonded together. For example, components such as the array 180, staircase 182 and digit line multiplexers 184 may be printed in an array die, while the other components are printed on a CMOS die. A set of wafer to wafer (W2W) contacts 178 couple the CMOS die to the array die. In some embodiments, certain components may be positioned above the memory array. For example, at least some of the sense amplifiers 174 may be positioned above the array 180.

The present disclosure will generally be described with respect to an example implementation where two chips are used, however other implementations using more or fewer chips may also be used, and other arrangements of components between chips may be used.

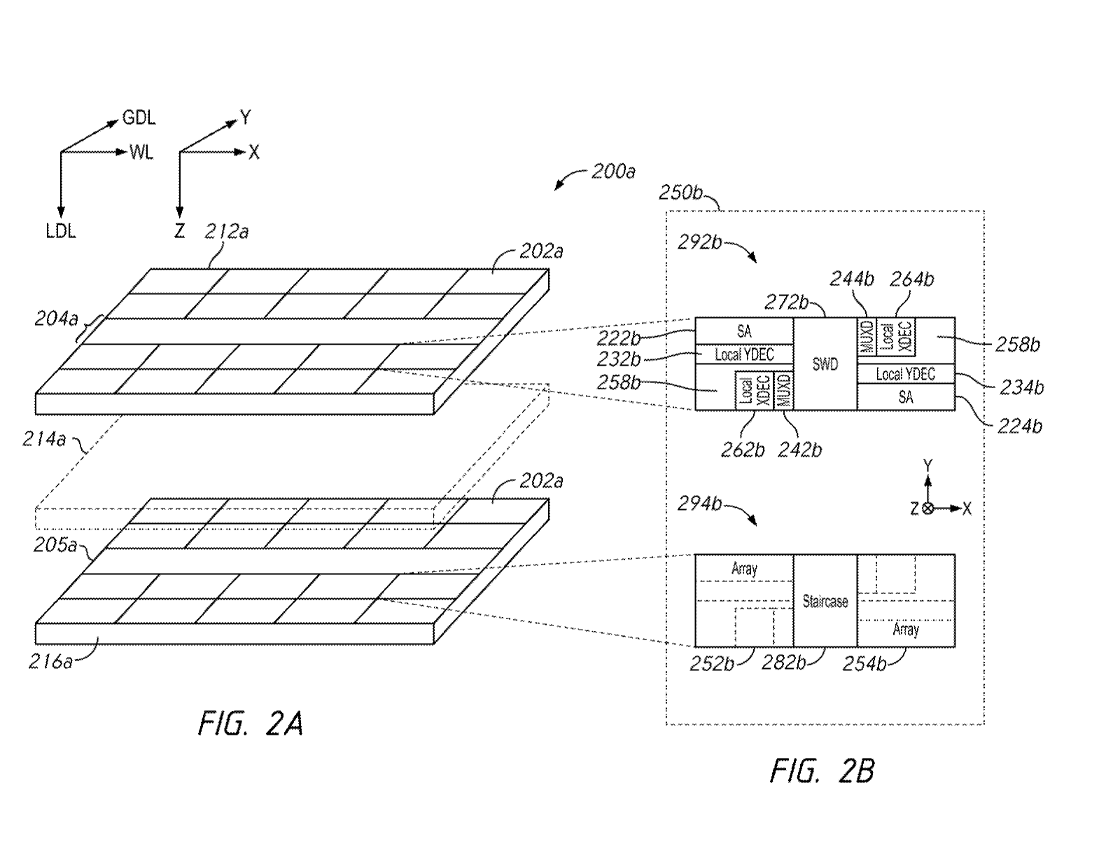

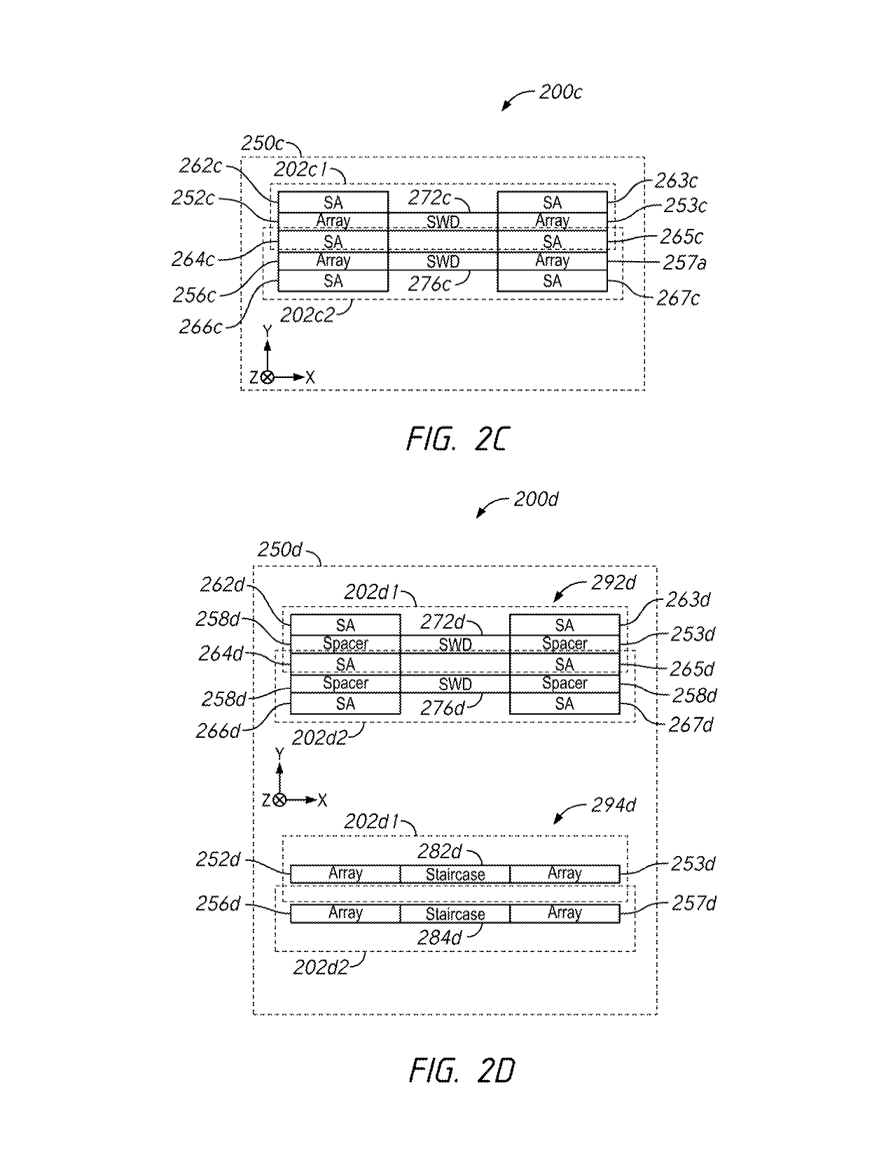

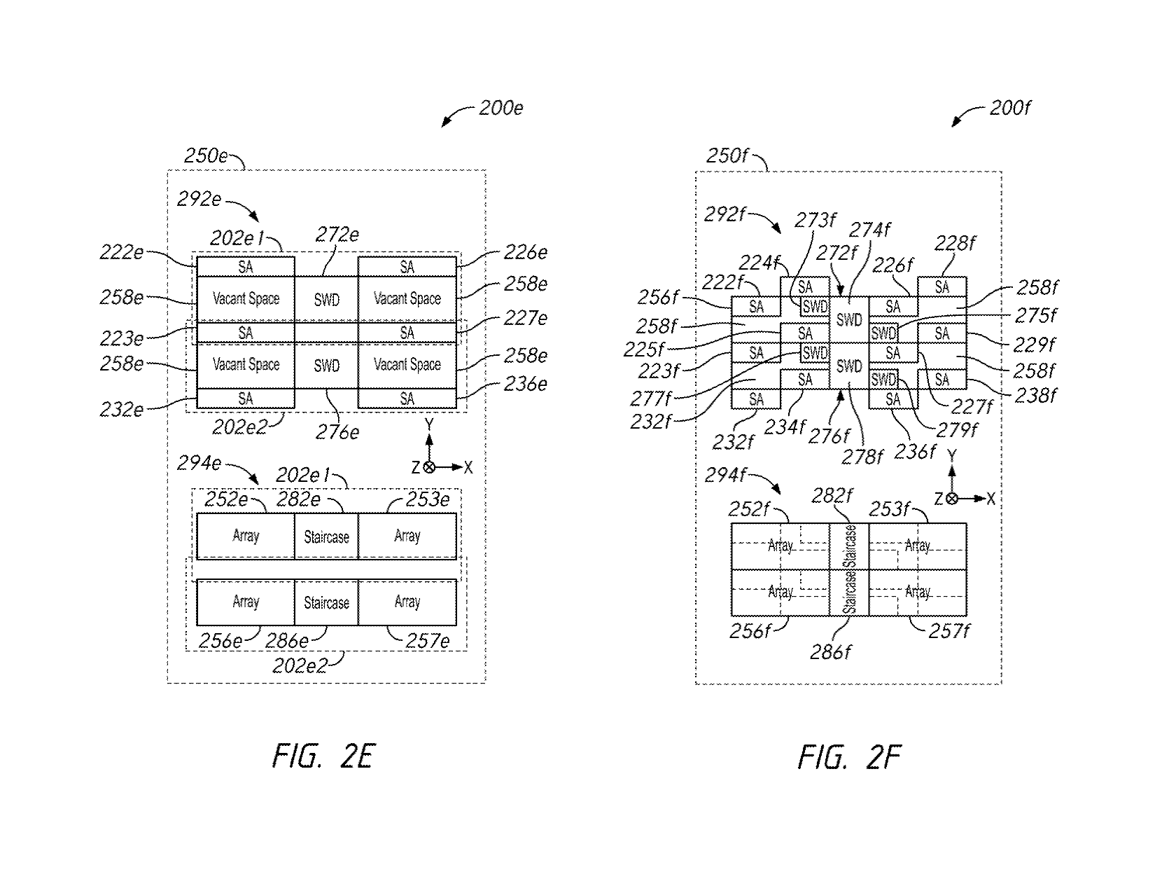

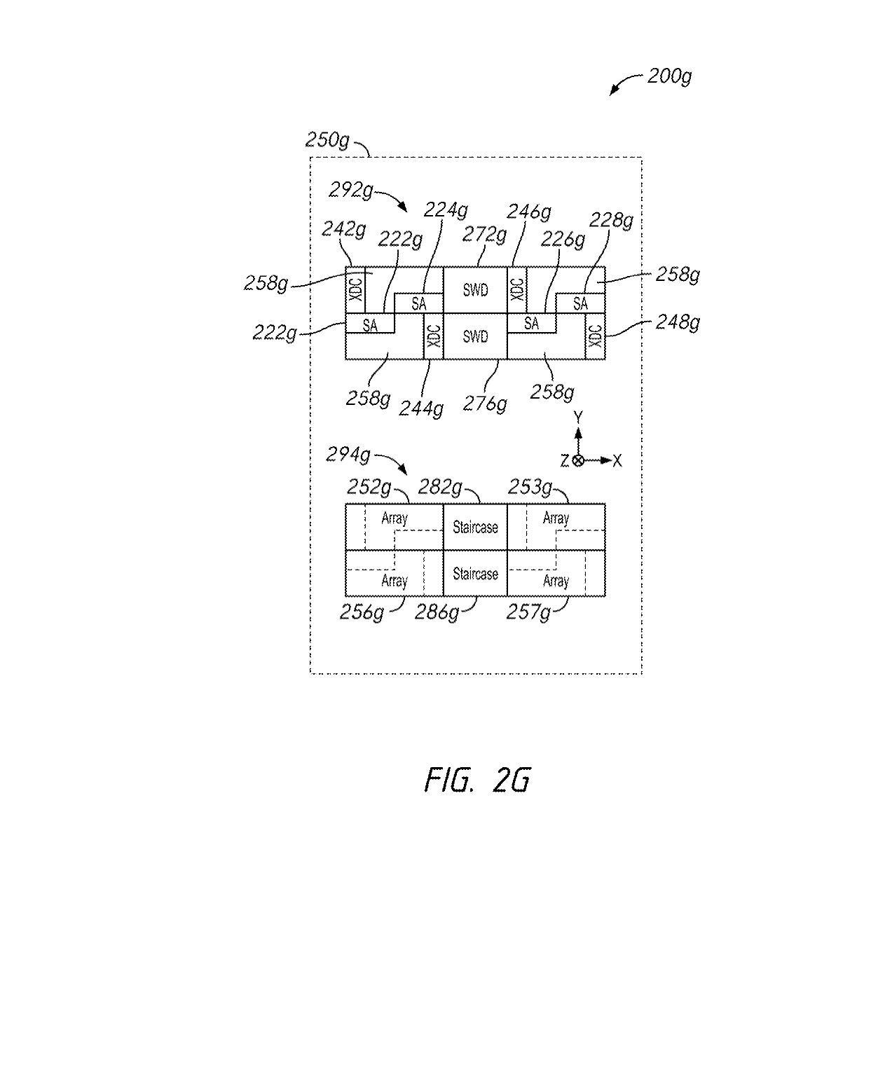

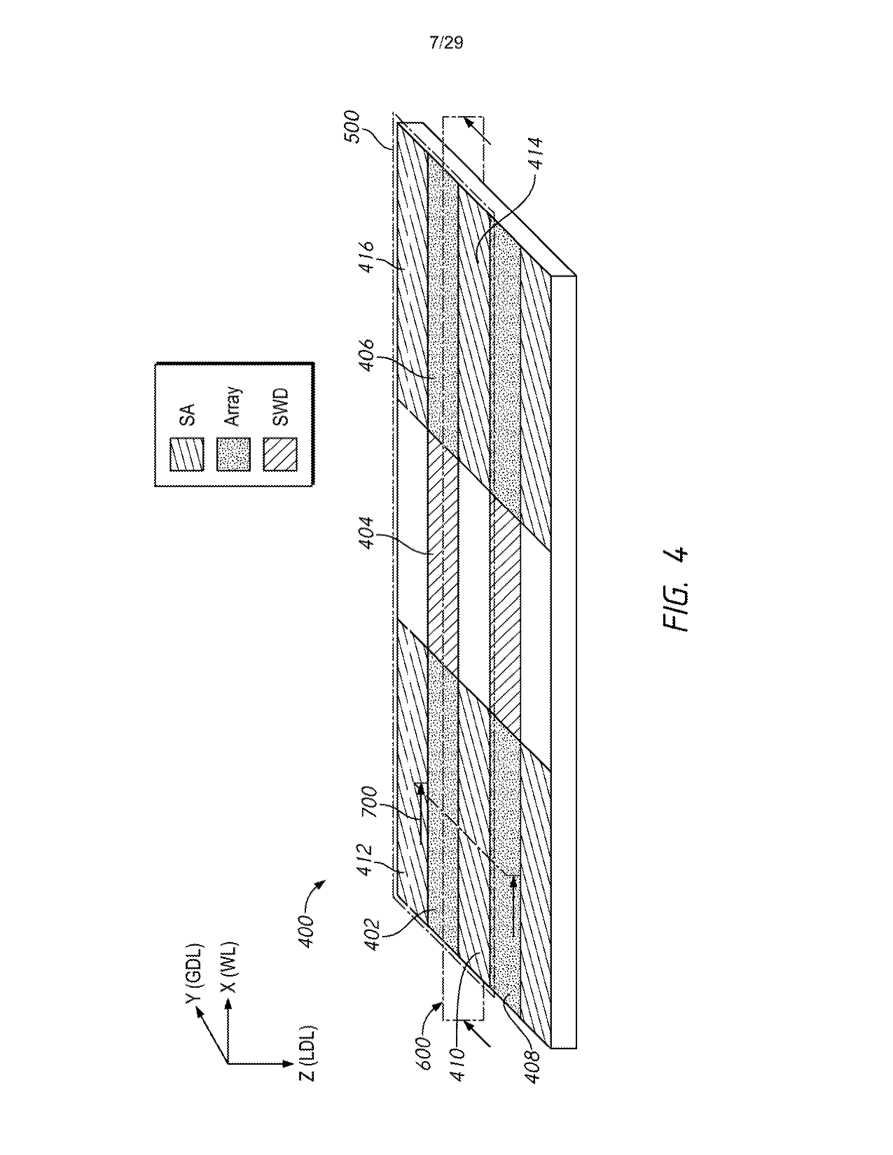

FIG. 2A is a perspective drawing of a memory device according to some embodiments of the present disclosure. FIGS. 2B-2G show example ‘top down’ views of different example layouts of a memory quilt of a memory device according to some example embodiments of the present disclosure. The memory device 200a of FIG. 2A and the example quilts 250b-250g of FIGS. 2B-2G may, in some embodiments, represent a layout which implements the 3D memory device 100 of FIG. 1. FIG. 2A shows an overall view of a memory device 200a and how different memory sections or ‘quilts’ and their supporting circuitry may be laid out across the device 200a. FIGS. 2B-2G each show a different example implementation of how the components of a quilt may be organized. The different example layouts of FIGS. 2B-2G are explained in more detail in FIGS. 4-20.

FIG. 2A shows a 3D memory device 200a (e.g., 100 of FIG. 1) which includes a number of memory quilts 202a, each of which includes one or more portions of memory array, such as memory patches, as well as circuitry which supports the operation of those patches such as sense amplifiers SAs (e.g., 174 of FIG. 1) and sub-word line drivers SWDs (e.g., 172 of FIG. 1). The memory quilts 202a are tiled in the x-y plane of the memory device 200a. The memory quilts 202a include sections of the memory array such as one or more array patches. The memory quilts 202a may be referred to as quilts herein, because when considered in the xy plane the repeating patterns of circuits tiled across surface of the device are evocative of quilt patches. The memory device 200a also includes a peripheral region 204a and 205a, which does not have memory quilts 202a tiled across it. The peripheral regions 204a and 205a may include other circuits which are used in the operation of the memory such as the command circuit 114 of FIG. 1, the input and output circuits 116-122 of FIG. 1 and other components.

In the example implementation of FIG. 2A, the memory device 200a is formed from two die, a first die 212a and a second die 216a. The first die 212a is stacked on top of the second die 216a in the z-direction. The first die 212a may be a die which includes various circuits and components which operate the memory device 200a, while the second die 216a may be an array die which includes the memory cells, word lines, local digit lines and global digit lines. As described in more detail herein, the second die 216 may also include components which are used to couple components together such as the staircase, and other components which aid the operation of the device 200a such as capacitors (e.g., 110 of FIG. 1), and/or multiplexers (e.g., 184 of FIG. 1). The first die 212a may generally be referred to as a CMOS die and the second die 216a may generally be referred to as an array die.

The two die 212a and 216a may be bonded together. For example, wafer-to-wafer (W2W) bonding may be used. A bottom surface in the z direction of the first die 212a may have one or more connection points such as bumps. A top surface in the z direction of the second die 216a may have corresponding connection points such as bumps. When the two die are bonded, the connection points may be electrically coupled between corresponding bumps. In some embodiments, an optional bonding layer 214a between the two die 214a and 216a may be used.

In the example of FIG. 2, the peripheral region 204a and 205a extends across a width of the two die 212a and 216a in an x-direction, and a portion of the surface of the dice 212a and 216a in the y-direction. The peripheral regions 204a and 205a are generally centered in the device along the y-axis. The peripheral region 204a is positioned on the CMOS die 212a while the peripheral region 205a is positioned on the array die 214a. The two peripheral regions 204a and 205a may generally be aligned with each other in the xy plane when the device 200a is stacked together.

The device 200a has memory quilts or memory sections 202a positioned in a grid layout above in the +y direction and below in the -y direction the edges of the peripheral regions 204a and 205a. Other arrangements of the peripheral region 204a/205a and quilts 202a may be used in other example embodiments. FIG. 2 shows a simplified view with a 2x5 grid of quilts 202a on either side of the peripheral region 204a and 205a. However, more or fewer quilts 202a, or different arrangements of quilts 202a may generally be used in other example embodiments. For example, an implementation of a memory device may generally be expected to include many more quilts 202a than the twenty quilts illustrated in FIG. 2A.

Both die 212a and 216a have memory quilts 202a which have respective portions tiled across the surface of the two die. Each memory quilt 202a has a first portion on the first die 212a and a second portion on the second die 216a. The portions of a given memory quilt 202a across the two die are vertically stacked so they align in an x-y plane and are stacked in the z direction.

FIGS. 2B-2G each show a top-down view in the xy of a different example memory quilt layout. The different layouts 250b-250g of FIGS. 2B-2G respectively may each represent an implementation of the quilt 202a of FIG. 2A. Accordingly, each of the quilt layouts 250b-250g shows a first portion 292b-292g in the first die 212a and a second portion 294b-294g in the second die 216a. The first portion 292b-292g may include components such as sense amplifiers (e.g., 174 of FIG. 1), multiplexer drivers (e.g., 176 of FIG. 1), sub-word line drivers (e.g., 172 of FIG. 1), row decoders (e.g., 162 of FIG. 1), column decoders (e.g., 164 of FIG. 1), or combinations thereof. The second portions 294b-294g may include components such as memory array patches (e.g., 180 of FIG. 1), multiplexers (e.g., 184 of FIG. 1), capacitors (e.g., 110 of FIG. 1), or combinations thereof. While the FIGS. 2B-2G show a range of possible layouts, other example layouts may also be used with the 3D memory device 200a. For example, the example layout 250b of FIG. 2B is the only layout to show local row and column decoders, however local row and column decoders may also be used with other layouts such as 250f and/or 250g of FIGS. 2F and 2G respectively.

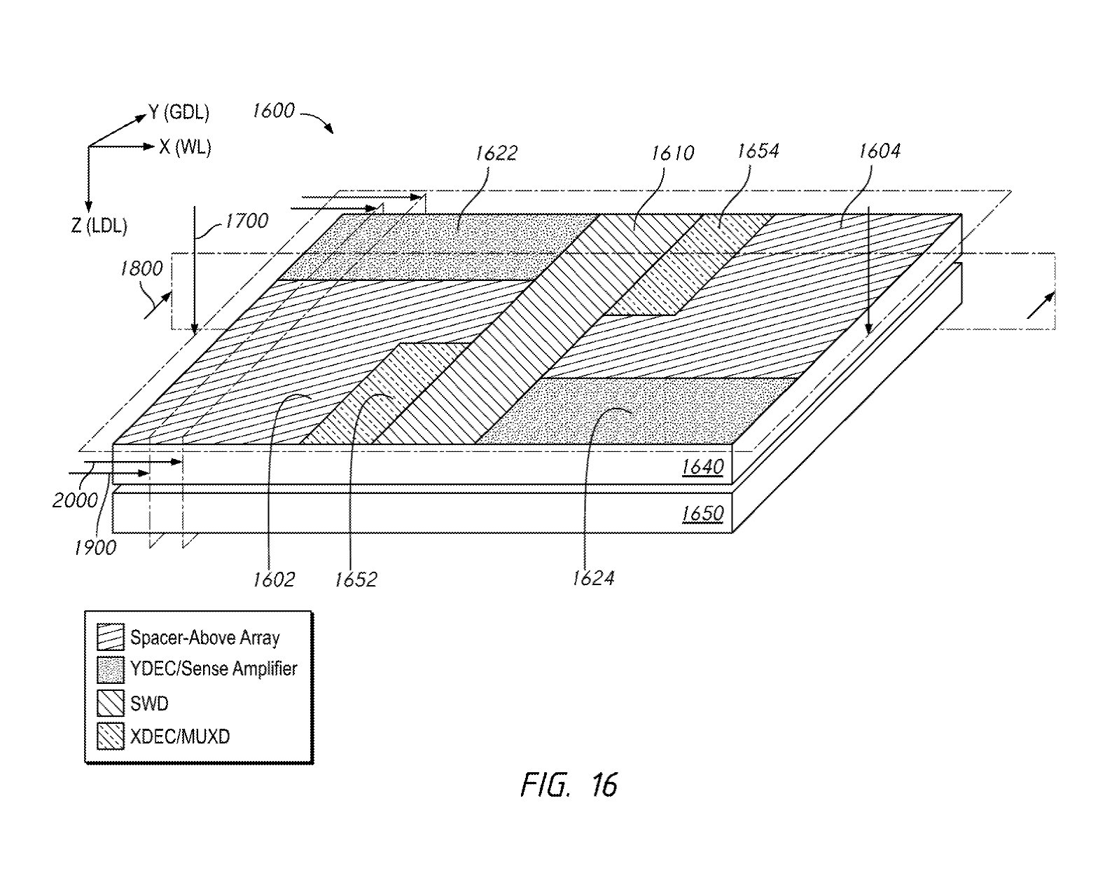

FIG. 2B shows an example quilt layout 250b with local row and column decoders positioned above the array patches. FIGS. 16-20 describe an example implementation of the layout 250b of FIG. 2B in more detail. The quilt layout 250b includes two memory patches 252b and 254b. Each memory patch 252b and 254b includes a 3D array of memory cells at the intersection of word lines and local digit lines, with the local digit lines coupled together by global digit lines. As well as the memory patches 252b and 254b, the quilt 250b also includes sense amplifier portions 222b and 224b, local column decoder 232b and 234b, multiplexer driver regions 242b and 244b, local row decoders 262b and 264b, sub-word line driver region 272b, and staircase region 282b. The sense amplifier portions 222b and 224b, local column decoder 232b and 234b, multiplexer driver regions 242b and 244b, local row decoders 262b and 264b, and sub-word line driver region 272b are located in the portion 292b on the first die (e.g., 212a). The array patches 252b and 254b and the staircase region 282b are located in the second portion 294b in the second die 216b.

The portion 292b of the quilt 250b in the first die 212b also includes spacer regions 258b, located on either side of the SWD region 272b. These spacer regions 258b are located above the memory array patches 252b-257b in the z direction. The spacer regions 258b may be regions of the first die 212b which are generally empty of circuits in some embodiments.

The patches 252b and 254b may be generally thought of as rectangular prisms, with a ‘top’ of the prism in the xy plane shown in the quilt layout 250b. Each patch 252b and 254b is also elongated along the z-axis, which is not shown in the ‘top down’ view of FIG. 2B. The two patches 252b and 254b may have generally the same dimensions as each other. The staircase region 282b is positioned between the two array patches 252b and 254b such that the staircase region 282b separates the two patches 252b and 254b along the x axis. Like the memory patches, the staircase region 282b may be thought of as a rectangular prism elongated in the x, y and z directions. The staircase may have generally the same height in the y axis as the two adjacent memory patches 252b and 254b.

The SWD region 272b is positioned above the staircase region 282b. The SWD region 272b may generally have the same x and y dimensions as the staircase region 282b. Each spacer region 258b is positioned above a corresponding one of the array patches 252b and 254b. Each of the patches 252b and 254b has an associated sense amplifier region, local column decoder, multiplexer driver region, and local row decoder. For example, the array patch 252b is associated with sense amplifier region 222b, local column decoder 232b, multiplexer driver region 242b and local row decoder 262b, while the array patch 254b is associated with sense amplifier region 224b, local column decoder 234b, multiplexer driver region 244b and local row decoder 264b.

The sense amplifiers are positioned above the patches 252b and 254b they are associated with. For example, the sense amplifier region 222b is above the patch 252b and the region 224b is above the patch 254b. Each of the sense amplifiers in the region is coupled to the respective global digit lines via vertical conductive elements run in the z direction down from the first die 212a to the second die 216a. Each sense amplifier may be coupled to a pair of global digit lines in the associated memory patch. In some embodiments, each sense amplifier region 222b/224b includes sense amplifiers arranged in a grid in the xy plane.

The sense amplifier regions 222b and 224b may be above a portion of the associated memory patch 252b and 254b. For example, the sense amplifier regions 222b/224b may generally have a same width in the x direction as the memory patches 252b/254b, but a shorter height in the y direction. The two sense amplifier regions 222b and 224b may be aligned with opposite edges of the memory patches. For example, an upper (in the y direction) edge of the sense amplifier region 222b is aligned with an upper edge of the memory patch 252b, while a lower edge of the sense amplifier region 224b is aligned with a lower edge of the memory patch 254b.

Each sense amplifier region 222b and 224b is associated with a respective local column decoder 232b and 234b (e.g., 164 of FIG. 1). During operations, the local column decoders 232b/234b use the column address to determine which global digit lines are coupled outside of the array along the global input/output (GIO) lines. The local column decoders 232b/234b are positioned over the array, next to the associated sense amplifier region. For example, the local column decoders 232b/234b may be along an ‘interior’ edge of the associated sense amplifier region. For example, the sense amplifier region 222b has an upper edge aligned with an upper edge of the patch 252b, and the associated local column decoder 232b along its lower edge.

The multiplexer drivers are positioned above the patches 252b and 254b they are associated with. For example, the multiplexer driver region 242b is above patch 252b and the multiplexer driver region 244b is above the patch 254b. An edge of the multiplexer driver regions 242b/244b is aligned with an edge of the SWD region 272b. For example, a right (in the x direction) edge of the multiplexer driver region 242b borders a left edge of the SWD region 272b, and a left edge of the multiplexer driver region 244b borders a right edge of the SWD region 272b. Each of the multiplexer driver regions has an associated local row decoder 262b/264b (e.g., 162 of FIG. 1) which is along an edge of the multiplexer driver region 242b/244b opposite the edge which borders the SWD region. For example, the multiplexer driver region 242b borders the SWD region 272b along its right edge and the associated local row decoder 262b along its left edge. The multiplexer driver regions 262b/264b are opposite the sense amplifier regions 222b/224b. For example, an upper edge of the sense amplifier region 222b is aligned with an upper edge of the patch 252b, and a lower edge of the multiplexer driver region 242b is aligned with a lower edge of the patch 252b.

The memory array patches 252b and 254b represent a piece of the 3D array. For example, each memory patch 252b-257b includes memory cells positioned at the intersection of word lines extending along the x axis and local digit lines extending in the z direction, which is into the plane of the page in the view of the quilt layout 250b. The local digit lines are coupled together in columns running in the y direction by global digit lines.

The word lines extend between two of the patches 252b and 254b. For example, a word line may be continuous across the width of the array patch 252b, through the staircase region 282b and across the width of the array patch 254b. The word lines intersect memory cells in the array patches, but not in the staircase region when passing through the staircase region 282b. For example, a word line intersects local digit lines and memory cells in the array patch 252b, then passes through the staircase region 282b without intersecting memory cells or LDLs, and then intersects LDLs and memory cells again in the array patch 254b. In other words, when considered along its length in the x direction, each word line has a gap where there are no coupled memory cells in the staircase region.

The SWD regions 272b and 276b include a number of SWDs, each of which is coupled to a respective word line which extends across both of the associated array sections 252b/254b through the staircase region 282b. For example, a first SWD in the region 272b is coupled to a first word line which extends across both array patches 252b and 254b, a second SWD in the region 272b is coupled to a second word line which extends across both array patches 252b and 254b, and so forth. The SWDs in the SWD region 272b are arranged in a grid, which has dimensions based on the number and arrangement of word lines in the adjacent memory patches 252b/254b. For example, if there are M word lines in the y-direction and N word lines in the z direction across the two patches 252b/254b for a total of NxM word lines in the two patches, then there will also be NxM SWDs in the region 272b. For example, the region 272b may have a grid of SWDs with M SWDs in the y-direction and N SWDs in the x direction. Along a given row in the x direction, each SWD couples to a different depth of word line in the z-direction using a connective element which extends along the z-direction to the associated word line.

The global digit lines in the array patches 252b and 254b are coupled to sense amplifiers in the associated sense amplifier portions 222b and 224b. The global digit lines in the patch 252b are coupled to sense amplifiers in the region 222b and the global digit lines in the patch 254b are coupled to sense amplifiers in the region 224b. Each sense amplifier is coupled to two global digit lines, both in the same patch. For example, each sense amplifier may be coupled to a pair of global digit lines which are adjacent to each other. In some embodiments, the pair of global digit lines may be stacked on top of each other in the z direction. For example, they may run in different metal layers. This layout may be referred to as a folded architecture, since the two digit lines run in a same direction (e.g., both run in the +y direction or in the -y direction) away from the sense amplifier. One of the pair of global digit lines is referred to as a ‘true’ global digit line or GDLT and the other is referred to as a ‘bar’ global digit line GDLB. Along the pair of GDLs, some local digit lines are coupled to GDLT and some are coupled to GDLB. During operations, one of the GDLT and GDLB is used to carry a signal to or from a memory cell and one is used as a reference by the coupled sense amplifier, depending on which word line is activated.

In some embodiments, the sense amplifiers within a region may be arranged in a grid. For example, if there are L global digit line pairs, there may be L sense amplifiers. The sense amplifiers may be arranged in a grid of I x J sense amplifiers, where I*J = L. The arrangement of the grid may be based, in part, on the size of the sense amplifiers in the x direction relative to the spacing of the GDL pairs in the x direction. For example, if a sense amplifier is generally as wide as four GDL pairs, then the sense amplifiers may be arranged in a grid with columns of four sense amplifiers each, with each of sense amplifier coupled to a different one of the GDL pairs than runs below that column.

In an example implementation, each of the memory patches 252b and 254b may include about 8Mbit of storage or about 8,192,000 memory cells. An example memory patch 252b has 8000 word lines arranged in a grid in the yz plane of 80 word lines in the y direction and 100 word lines in the z direction. The word lines are intersected by a grid in the xy plane of 1024x80 local digit lines with 1024 local digit lines in the x direction and 80 in the y direction. Each ‘column’ of 80 local digit lines is coupled to one or the other of a pair of associated global digit lines for a total of 1024 global digit line pairs or 2048 total global digit lines. The associated sense amplifier region 222b includes 1024 sense amplifiers, each coupled to a pair of global digit lines. The sense amplifiers may be arranged in an xy grid of 256 sense amplifiers in the x direction and 4 sense amplifiers in the y direction. Each sense amplifier is coupled to the associated GDL pair by a vertical (in the z direction) element running down to the GDL running below the sense amplifier. The SWD region 272b includes 8000 SWDs, one for each word line, arranged in a grid in the xy plane of 80 SWDs in the y direction and 100 in the x direction. The multiplexer driver region 242b includes half of the total multiplexer drivers, arranged in a column in the y direction. For example, if each multiplexer driver is coupled to a single LDL, then there are 80 total multiplexer drivers, 20 of which are in the region 242b (the other 20 are in region 244b).

FIGS. 2C shows a top down view in an xy plane of an example layout 250c where the memory device 200a is on a single die. Unlike the other example embodiments described herein, the example embodiment of the layout 200c shows a memory device which is implemented in a single die. Accordingly, unlike other described embodiments, components such as the sense amplifiers, sub-word line drivers, and array patches are all in a single die. The example layout 250c shows a ‘top down’ view in the xy plane of two adjacent sections 202c1 and 202c2 each of which may implement one of the sections 202a of FIG. 2A. The two sections 202c1 and 202c2 are adjacent to each other in the y direction. The example layout 250c only shows two sections 202c1 and 202c2, however those sections may border other sections which are not shown in the inset 250c.

Each section 202c1 and 202c2 includes a pair of memory patches, four sense amplifier (SA) regions, and a sub word line driver (SWD) and staircase region. Section 202c1 includes memory patches 252c and 253c (also referred to as memory array patches or array patches), SA regions 262c, 263c, 264c, and 265c, and SWD and staircase region 272c. Section 202c2 includes memory patches 256c and 257c (also referred to as memory array patches or array patches), SA regions 264c, 265c, 266c, and 267c, and SWD and staircase region 276c.

The example layout of section 202c1 is described in detail. Since each section may be generally similar, the layout of section 202c2 is not described in detail. The section 202c1 includes a first array patch 252c and a second array patch 253c. Patches 252c and 253c are generally elongated along the x axis, and are generally longer along the x axis than they are tall along the y axis. Each patch 252c and 253c is also elongated along the z-axis, which is not shown in the ‘top down’ view of FIG. 2C. The SWD and staircase region 272c is positioned between the two array patches 252c and 253c such that the SWD and staircase region 272c separates the two patches 252c and 253c along the x axis. The SWD and staircase region 272c may be elongated in the x direction and may be longer in the x direction than it is tall in the y direction. In some embodiments, the height of the SWD and staircase region 272c in the y-axis may approximately match the height of the two adjacent array patches 252c and 253c.

Each memory array patch 252c and 253c is bordered by two sense amplifier regions positioned above and below array patches 252c and 253c in the y-direction. For example, the SA region 262c is above in a +y direction the array patch 252c and the SA region 264c is below in a -y direction the array patch 252c. Similarly, the SA region 263c is above the array patch 253c in a +y direction and the SA region 265c is below the array patch 253c in a -y direction. The SA regions 262c-265c may generally be elongated in the x direction. The SA regions 262c-265c may generally have the same length in the x-axis as the array patches 252c and 253c they are adjacent to. The SA region 262c is separated from the SA region 263c by a gap in the x direction, and the SA region 264c is separated from the SA region 265c by a gap in the x-direction.

The memory array patches 252c-257c represent a piece of the 3D array. For example, each memory patch 252c-257c includes memory cells positioned at the intersection of word lines extending along the x axis and local digit lines extending in the z direction, which is into the plane of the page in the view of the layout 250c. The local digit lines are coupled together in columns running in the y direction by global digit lines.

The word lines in a section 202c1 or 202c2 extend continuously between the two array patches 252c/253c or 256c/257c. For example, a word line may be continuous across the width of the array patch 252c, through the staircase region 272c and across the width of the array patch 253c. The word line intersects memory cells in the array patches 252c and 253c, but not in the staircase region 272c when passing underneath the SWDs. For example, a word line intersects local digit lines and memory cells in the array patch 252c, then passes through the staircase region 272c without intersecting memory cells or LDLs, and then intersects LDLs and memory cells again in the array patch 253c. In other words, when considered along its length in the x direction, each word line has a gap where there are no coupled memory cells in the staircase region 272c.

The SWD and staircase regions 272c and 276c include a number of SWDs, each of which is coupled to a respective word line which extends across both of the associated array patches 252c/253c or 256c/257c. For example, a first SWD in the SWD region 272c is coupled to a first word line which extends across both array patches 252c and 253c, a second SWD in the SWD region 272c is coupled to a second word line which extends across both array patches 252c and 253c, and so forth. The SWDs in the SWD region 272c or are arranged in a grid, which has dimensions based on the number and arrangement of word lines in the adjacent memory patches 252c/253c or 256c/257c. For example, if there are M word lines in the y-direction and N word lines in the z direction across the two patches 252c/253cor 256c/257c for a total of NxM word lines in each section 202c1 or 202c2, then there will also be NxM SWDs in the SWD region 272c or 276c. For example, the region 272 may have a grid of SWDs with M SWDs in the y-direction and N SWDs in the x direction. Along a given row in the x direction, each SWD couples to a different depth of word line in the z-direction using a connective element which extends along the z-direction to the associated word line.

The sense amplifier regions 262c-267c each include a number of sense amplifiers, each of which is coupled to a respective global digit line in the adjacent array patches 252c-257c. In the implementation of FIG. 2C, the sense amplifiers in the SA regions 262c-267c may generally couple to alternate global digit lines. For example, the sense amplifiers in the SA region 262c may couple to ‘even’ global digit lines in the array patch 252c, while the sense amplifiers in the SA region 264c may couple to ‘odd’ global digit lines in the array patch 252c.

The two sections 202c1 and 202c2 may share components. For example, the sense amplifier regions 264c and 265c include sense amplifiers which are coupled to global digit lines in both of the adjacent memory sections 202c1 and 202c2. For example, the sense amplifier region 264c is coupled to global digit lines in both array patches 252c and 256c and the sense amplifier region 265c is coupled to global digit lines in both array patches 253c and 257c. The sense amplifiers in the SA region 264c may couple to odd global digit lines in both of the adjacent array patches 252c and 256c. Similarly, the sense amplifiers 262c may couple to even global digit lines in the array patch 252c and a different array patch adjacent on the other side, not shown in the layout 250c. The sections which are on the edge of the die 200a with no adjacent section in the y-direction may have a region of sense amplifiers which are not shared, but instead only couple to global digit lines in the one adjacent patch.

In an example implementation, each of the memory patches 252c-257c may include about 1Mbit of storage or about 1,024,000 memory cells. A given section 202c1 or 202c2 has 1000 word lines arranged in a grid in the yz plane of 10 word lines in the y direction and 100 in the z direction. Each associated sense amplifier region 262c-267c includes about 512 sense amplifiers coupled to half of the global digit lines which cross the array section 252, for a total of 1024 global digit lines in each memory section 252c-257c. The SWD and staircase regions 272cand 276c each include 1000 SWDs, arranged in a grid in the xy plane of 10 SWDs in the y direction and 100 in the x direction.

FIG. 2D shows a top down view of an example layout 250d. The example layout 250d is generally similar to the layout 250c of FIG. 2C, except that the layout 250d is split across two die (e.g., 212a and 216a of FIG. 2A). An implementation of the example layouts of FIGS. 2C and 2D are described in more detail in FIGS. 4-7. The layout 250d shows an example of two sections 202d1 and 202d2. The inset shows a first portion 292d which is in the first die 212a of FIG. 2A and a second portion 294d which is in the second die 216a of FIG. 2A. The layout of the sections 202d1 and 202d2 may be generally similar to the sections 202c1 and 202c2 of FIG. 2C except that in FIG. 2D, the sections 202d1 and 202d2 are split across two dice. In particular, the sense amplifier regions 262d-267d and SWD regions 272d and 276d are located in the portion 292d on the first die, while the array patches 252d-257d and the staircase regions 282d and 284d are located in the portion 294d on the second die.

The portion 292d includes spacer regions 258d, located on either side of the SWD regions 272d and 276d and between the SA regions 262d-267d. These spacer regions 258d are located above the memory array patches 252d-257d in the z direction. The spacer regions 258d may be regions of the first die 212d which are generally empty of circuits in some embodiments.

FIG. 2E shows an example layout 250e which is generally similar to the embodiment of FIG. 2D, except that in FIG. 2E an increased number of memory cells per patch are used. The inset 250e shows a ‘top down’ view in the xy plane of two adjacent sections 202e1 and 202e2. The sections 202e1 and 202e2 represent examples of two of the sections 202e. The two sections 202e1 and 202e2 are adjacent to each other in the y direction. The inset 250e only shows two sections 202e1 and 202e2, however those sections may border other sections which are not shown in the inset 250e.

Each section 202e1 and 202e2 includes a pair of memory patches, four sense amplifier regions, and a sub word line driver and staircase region. Section 202e1 includes memory patches 252e and 253e, SA regions 222e, 226e, 223e, and 227e, SWD region 272e and staircase region 282e. The sense amplifier regions 222e-227e and SWDs 272e are located in a first portion 292e on a first die (e.g., 212a of FIG. 2A), while the staircase regions 282e and array patches 252e-257e are located in a second portion 294e on a second die (e.g., 214e of FIG. 2E). Section 202e2 includes memory patches 256e and 257e, SA regions 223e, 227e, 232e, and 236e, SWD region 276e and staircase region 286e. The sense amplifier regions 223e-236e and SWDs 276e are located in the first die while the staircase regions 286e and array patches 256e and 257e are located in the second die 256e.

The portion 292e includes spacer regions 258e, located on either side of the SWD regions 272e and 276e and between the SA regions 222e and 223e, and between the SA regions 226e and 227e. These spacer regions are located above the memory array patches 252e-257e in the z direction. The spacer regions 258e may be regions of the first die 212e which are generally empty of circuits in some embodiments.

The example layout of section 202e1 is described in detail. Since each section may be generally similar, the layout of section 202e2 is not described in detail. The section 202e1 includes a first array patch 252e and a second array patch 253e. Patches 252e and 253e are generally elongated along the x axis, and are generally longer along the x axis than they are tall along the y axis. Each patch 252e and 253e is also elongated along the z-axis, which is not shown in the ‘top down’ view of FIG. 2E. The staircase region 282e is positioned between the two array patches 252e and 253e such that the staircase region 282e separates the two patches 252e and 253e along the x axis. The staircase region 282e may be elongated in the x direction and may be longer in the x direction than it is tall in the y direction. In some embodiments, the height of the SWD and staircase region 282e in the y-axis may approximately match the height of the two adjacent array patches 252e and 253e.

A SWD region 272e is positioned above the staircase region 282e. The SWD region 272e may generally have the same x and y dimensions as the staircase region 282e. Each spacer region 258e positioned above the array patches 252e and 253e is bordered by two sense amplifier regions positioned above and below the spacer regions 258e in the y-direction. For example, the SA region 222e is above in a +y direction the spacer 258e above the array patch 252e and the SA region 223e is below in a -y direction the spacer 258e above the array patch 252e. Similarly, the SA region 226e is above spacer 258e above array patch 253e in a +y direction and the SA region 236e is below the spacer 258e above the array patch 253e in a -y direction. The SA regions 222e-227e may generally be elongated in the x direction. The SA regions 222e-227e may generally have the same length in the x-axis as the array patches 252e and 253e they are associated with. The SA region 222e is separated from the SA region 226e by a gap in the x direction, and the SA region 223e is separated from the SA region 227e by a gap in the x-direction. The gaps may be the width of the SWD region 272e.

The memory array patches 252e-257e represent a piece of the 3D array. For example, each memory patch 252e-257e includes memory cells positioned at the intersection of word lines extending along the x axis and local digit lines extending in the z direction, which is into the plane of the page in the view of the layout 250e. The local digit lines are coupled together in columns running in the y direction by global digit lines.

The word lines in a section 202e1 or 202e2 extend continuously between the two array patches 252e and 253e or 256e and 257e. For example, a word line may be continuous across the width of the array patch 252e, through the staircase region 282e and across the width of the array patch 253e. The word line intersects memory cells in the array patches, but not in the staircase region when passing underneath the SWDs. For example, a word line intersects local digit lines and memory cells in the array patch 252e, then passes through the staircase region 282e without intersecting memory cells or LDLs, and then intersects LDLs and memory cells again in the array patch 253e. In other words, when considered along its length in the x direction, each word line has a gap where there are no coupled memory cells in the staircase region.

The SWD regions 272e and 276e include a number of SWDs, each of which is coupled to a respective word line which extends across both of the associated array sections 252e/253e or 256e/257e through the staircase region 282e and 286e. For example, a first SWD in the region 272e is coupled to a first word line which extends across both array patches 252e and 253e, a second SWD in the region 272e is coupled to a second word line which extends across both array patches 252e and 253e, and so forth. The SWDs in the SWD region 272e or are arranged in a grid, which has dimensions based on the number and arrangement of word lines in the adjacent memory patches 252e/253e or 256e/257e. For example, if there are M word lines in the y-direction and N word lines in the z direction across the two patches 252e/253e or 256e/257e for a total of NxM word lines in each section 202e1 or 202e2, then there will also be NxM SWDs in the region 272e or 276e. For example, the region 272e may have a grid of SWDs with M SWDs in the y-direction and N SWDs in the x direction. Along a given row in the x direction, each SWD couples to a different depth of word line in the z-direction using a connective element which extends along the z-direction to the associated word line.

The sense amplifier regions 222e-236e each include a number of sense amplifiers, each of which is coupled to a respective global digit line in the associated array sections 252e-257e below the spacer regions 258e that the sense amplifier region 222e-236e is adjacent to. In the implementation of FIG. 2E, the sense amplifiers in a regions 222e-236e may generally couple to alternate global digit lines. For example, the sense amplifiers in the region 262e may couple to ‘even’ global digit lines in the array section 252e, while the sense amplifiers in the region 264e may couple to ‘odd’ global digit lines in the array section 252e.

The two sections 202e1 and 202e2 may share components. For example, the sense amplifier regions 223e and 227e include sense amplifiers which are coupled to global digit lines in both of the memory patches 252e and 256e under the spacer regions 258e adjacent to those sense amplifier regions 223e and 227e. For example, the sense amplifier region 223e is coupled to global digit lines in both array sections 252e and 256e and the sense amplifier region 227e is coupled to global digit lines in both array patches 253e and 257e. In an example implementation, the sense amplifiers in the region 223e may couple to odd global digit lines in both of the array patches 252e and 256e. Similarly, the sense amplifiers 222e may couple to even global digit lines in the array section 252e and a different array patch adjacent on the other side, not shown in the layout 250e. The sections which are on the edge of the device with no adjacent section in the y-direction may have a region of sense amplifiers which are not shared, but instead only couple to global digit lines in the one adjacent patch.

In an example implementation, each of the memory patches 252e-257e may include about 8Mbit of storage or about 8,192,000 memory cells. An example memory patch 252e has 8000 word lines arranged in a grid in the yz plane of 80 word lines in the y direction and 100 word lines in the z direction. Each associated sense amplifier region 222e and 223e includes about 512 sense amplifiers coupled to half of the global digit lines which cross the array patch 252e, for a total of 1024 global digit lines in the memory patch 252e. The SWD region 272e includes 8000 SWDs, one for each word line, arranged in a grid in the xy plane of 80 SWDs in the y direction and 100 in the x direction.

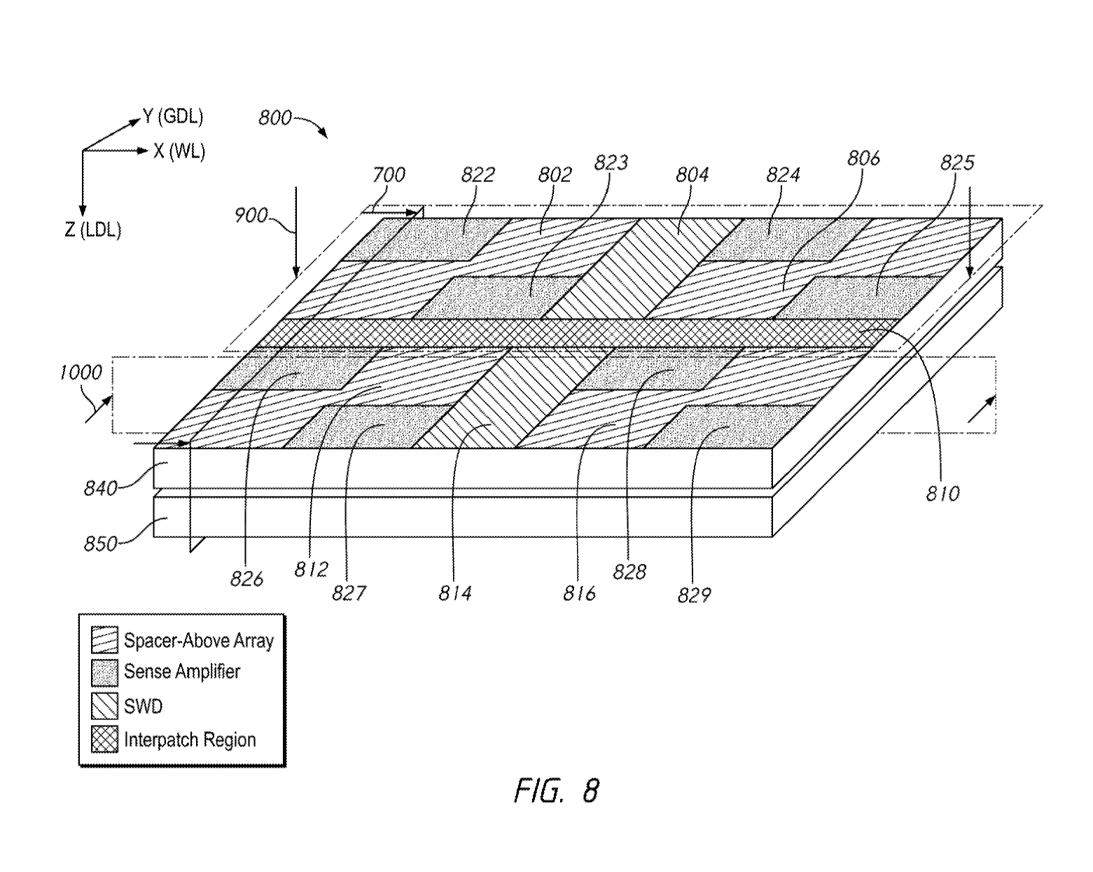

FIG. 2F shows a top down view in an xy plane of an example layout 250f with sense amplifiers positioned above the array patches. The layout 250f may be generally similar to layout 250e of FIG. 2E, except that the layouts 250f are different than the layouts 250e of FIG. 2E. An example implementation of the layout of FIG. 2F is described in more detail in FIGS. 8-11.

The portion 294f has dotted lines on the array patches 252f-257f to represent the portions of the array patches 252f-257f which have circuit elements above them (in the z-direction). Since the elements in the portion 292f are in a different die than the elements in the portion 294f, the circuit elements can occupy overlapping regions of space in the xy plane with the array patches 252f-257f without interfering with the operation or layout of the patches 252f-257f. By moving components in the first die 212a of FIG. 2A over the array patches 252f-257f, the overall xy dimensions of the layout 250f may be reduced compared to the 250e of FIG. 2E while maintaining a same number of memory cells per section. This may allow a reduction in the overall size of the die and/or an increase in the amount of storage (e.g., the number of memory cells) on the die.

In the sections 202f, the sense amplifiers 222f-238f are positioned over one of the two memory patches they are associated with. Each sense amplifier region may be separated into two portions, one of which is over one of the two patches that region is associated with and the other of which is over the other of the two patches that region is associated with. For example, the sense amplifier region associated with the patches 252f and 256f is divided into two portions 223f and 225f. The portion 223f is positioned above the array patch 256f and the portion 225f is positioned above the array patch 252f. For example, compared to an interpatch region which runs along the border between the two patches 252f and 256f and the two spacer regions 258f above them, the sense amplifier portion 223f is displaced in a -y direction from the interpatch region while the sense amplifier portion 225f is displaced in a +y direction from the interpatch region. In other words, using ‘upper’ and ‘lower’ to refer to the +y and -y directions for this example, the upper edge of the sense amplifier region 223f is along the interpatch region while the lower edge of the sense amplifier region 226f is along the interpatch region.

The global digit lines may run through the array patches in the y direction to the interpatch region, where they have vertical elements extending in the z direction from the interpatch region in the die 216f to the interpatch region in the first die. In the first die 212 they couple to horizontal elements running in the y direction which couple them to the associated sense amplifier portion.

The two portions may divide the sense amplifier region in half. For example, if there are 512 sense amplifiers for each region, then each sense amplifier portion 222f-238f includes 256 sense amplifiers. In other words, a first half of the even or odd digit lines may couple to a first sense amplifier portion while a second half of the even or odd digit lines may couple to a second sense amplifier portion. For example, considering the patch 252f, a first half of the even GDLs couple to the portion 222f, a second half of the even GDLs couple to the portion 224f, a first half of the odd digit lines couple to the portion 223f, and a second half of the odd digit lines couple to the portion 225f. The portions 222f and 225f are above the memory patch 252f, but the portions 224f and 223f are not above the patch 225f, but are above adjacent patches instead. In this manner, in a given patch the first half of the GDLs digit lines have one of even or odd GDLs coupled to sense amplifiers which are over the patch while the other of the even or odd GDLs are coupled to sense amplifiers which are not over the patch and the second half of the GDLS have the other of even or odd GDLs coupled to sense amplifiers which are over the patch while the one of the even or odd GDLs are coupled to sense amplifiers which are not over the patch.

In the example of FIGS. 2F, some of the SWDs are also positioned above the patches 252f-257f. For example, each SWD region 272f and 276f may be divided into a central region which is between the two associated patches, and side portions which are each above a respective one of the associated portions. For example, the SWD region 272f includes a central portion 274f, a first side portion 273f which is above patch 252f and a second side portion 275f which is above patch 253f. The SWD region 276f includes a central portion 278f, a first portion 277f which is above the first which is above the patch 256f and a second portion 279f which is above the patch 257f. The two portions which are above the patches may be aligned with different edges of the central portion. For example the portion 273f has a top edge (in the y direction) aligned with the top edge (in the y direction) of the central portion 274f, while the portion 275f has a lower edge (in the y direction) aligned with the lower edge (in the y direction) of the central portion 274f. In some embodiments, the portions which are over the array patch may be smaller than the central portion which is over the staircase region.

The SWDs which are in the portions above the patch 252f-257f may still connect to the word line through the staircase region. For example, each of the SWDs in the SWD region 272f may be coupled to their respective WLs by vertical conductive elements which run in the z direction in the staircase region 282f. The SWDs in the central portion 274f which is above the staircase region 282f may be coupled directly to the vertical conductive elements. The SWDs in the regions 273f and 275f are coupled by horizontal conductive elements in the x direction.

In some example embodiments, like the layout sown in FIG. 2F, both sense amplifiers and SWDs may be positioned over the patches. In some example embodiments, the sense amplifiers 222f-238f may be positioned over the array, but the SWDs may be in a central region between the patches similar to FIG. 2F. In some example embodiments, some of the SWDs may be over the patches, but the sense amplifiers may be positioned in the interpatch region similar to FIG. 2G.

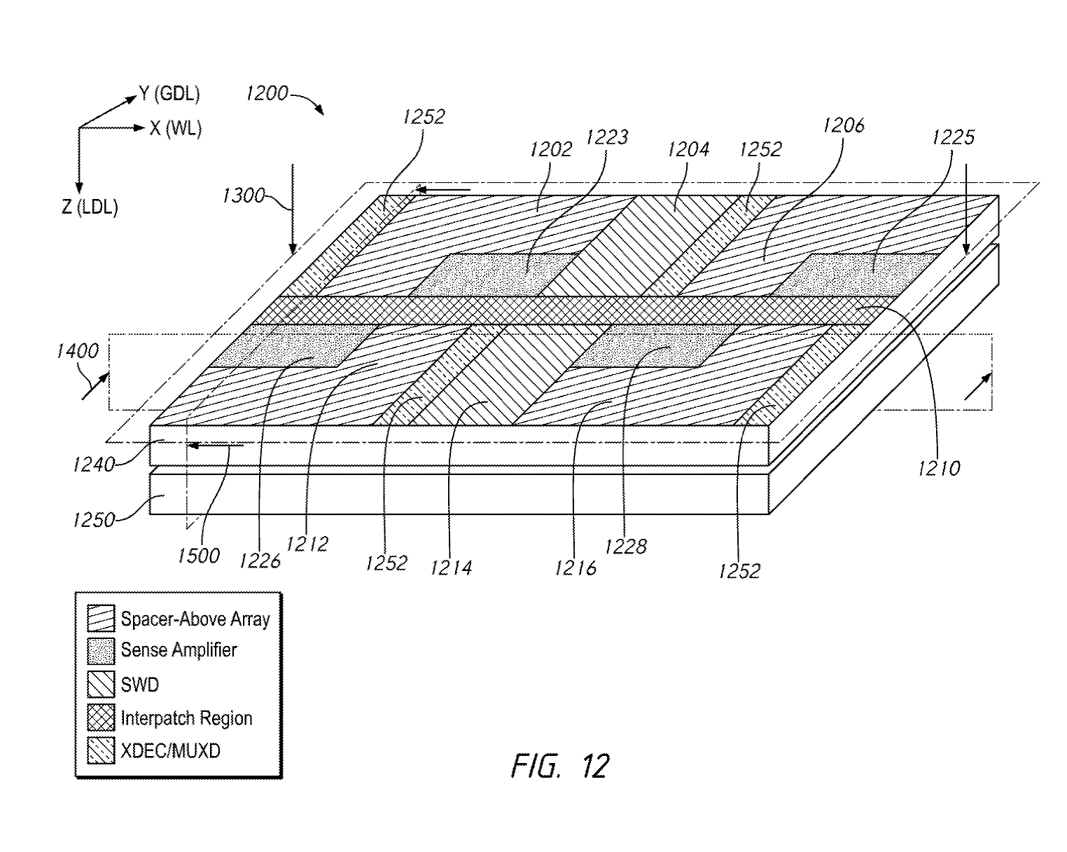

FIG. 2G shows an example layout 250g with a ‘top down’ view in the xy plane of a quilt. The quilt layout 250g includes sense amplifiers along one edge of the memory patch. An example implementation of the layout 250g is described in more detail in FIGS. 12-15. The quilt layout 250g includes four memory array patches 252g, 253g, 256g and 257g. Each memory patch 252g-257g includes a 3D array of memory cells at the intersection of word lines and local digit lines, with the local digit lines coupled together by global digit lines. The quilt layout 250g also includes sense amplifier portions 222g-228g, sub-word line driver regions 272g and 286g, staircase regions 282g and 286g, and row decoder/multiplexer driver regions 242g-248g. The sense amplifier regions 223g-236g, row decoder/multiplexer drivers 242g-248g, and SWDs 276g are located in the first die 212a of FIG. 2A while the staircase regions 286g and array patches 256g and 257g are located in the second die 216a of FIG. 2A.

The portion 292g of the quilt layout 250g in the first die also includes spacer regions 258g, located on either side of the SWD regions 272g and 276g. These spacer regions 258g are located above the memory array patches 252g-257g in the z direction. The spacer regions 258g may be regions of the first die 212g which are generally empty of circuits in some embodiments.

The example layout of the quilt 250g may be used for each of the quilts 202aof FIG. 2A. The patches 252g-257g are generally elongated along the x axis, and are generally longer along the x axis than they are tall along the y axis. Each patch 252g-257g is also elongated along the z-axis, which is not shown in the ‘top down’ view of FIG. 2A. The staircase region 282g is positioned between the two array patches 252g and 253g such that the staircase region 282g separates the two patches 252g and 253g along the x axis. The staircase region 282g may be elongated in the x direction and may be longer in the x direction than it is tall in the y direction. In some embodiments, the height of the SWD and staircase region 282g in the y-axis may approximately match the height of the two adjacent array patches 252g and 253g. In a similar fashion, the staircase region 286g separates the array patches 256g and 257g.

The SWD region 272g is positioned above the staircase region 282g. The SWD region 272g may generally have the same x and y dimensions as the staircase region 282g. The SWD region 276g is positioned above the staircase region 286g in a similar fashion. Each spacer region 258g is positioned above a corresponding one of the array patches 252g-257g. Each patch 252g-257g has a sense amplifier region and a row decoder/multiplexer driver region positioned above them.

The sense amplifiers are positioned above the patches 252g-257g. For example, the sense amplifier portion 222g is above the patch 256g, the portion 224g is above the patch 252g, the portion 226g is above the patch 257g and the portion 228g is above the patch 253g. Each of the sense amplifiers is coupled to the respective global digit lines via vertical conductive elements which run in an interpatch region between the two patches. The portions may be offset from each other in a y direction. For example, the portion 222g may be a row of sense amplifiers running from an upper left (e.g., in the +y and -x) corner of the patch 256g to roughly a center of the top (+y) border of the patch 256g. The portion 224g is a row of sense amplifiers running from roughly a center of the lower (-y) border of the patch 252g to a lower right (e.g., -y and +x) corner of the patch 252g. In other words, considering the interpatch region between patches 252g and 256g, the sense amplifier portion 222g is aligned with its top border along the interpatch region while the sense amplifier portion 224g is aligned with its lower border along the interpatch region.

The memory array patches 252g-257g represent a piece of the 3D array. For example, each memory patch 252g-257g includes memory cells positioned at the intersection of word lines extending along the x axis and local digit lines extending in the z direction, which is into the plane of the page in the view of the layout 250g. The local digit lines are coupled together in columns running in the y direction by global digit lines.

The word lines extend between two of the patches. For example, a word line may be continuous across the width of the array patch 252g, through the staircase region 282g and across the width of the array patch 253g. Another example word line may extend across the patch 256g, through the staircase region 286g, and through the array patch 257g. The word lines intersect memory cells in the array patches, but not in the staircase region when passing underneath the SWDs. For example, a word line intersects local digit lines and memory cells in the array patch 252g, then passes through the staircase region 282g without intersecting memory cells or LDLs, and then intersects LDLs and memory cells again in the array patch 253g. In other words, when considered along its length in the x direction, each word line has a gap where there are no coupled memory cells in the staircase region.

The SWD regions 272g and 276g include a number of SWDs, each of which is coupled to a respective word line which extends across both of the associated array sections 252g/253g or 256g/257g through the staircase region 282g and 286g. For example, a first SWD in the region 272g is coupled to a first word line which extends across both array patches 252g and 253g, a second SWD in the region 272g is coupled to a second word line which extends across both array patches 252g and 253g, and so forth. The SWDs in the SWD region 272g or are arranged in a grid, which has dimensions based on the number and arrangement of word lines in the adjacent memory patches 252g/253g or 256g/257g. For example, if there are M word lines in the y-direction and N word lines in the z direction across the two patches 252g/253g or 256g/257g for a total of NxM word lines in each section 202g1 or 202g2, then there will also be NxM SWDs in the region 272g or 276g. For example, the region 272g may have a grid of SWDs with M SWDs in the y-direction and N SWDs in the x direction. Along a given row in the x direction, each SWD couples to a different depth of word line in the z-direction using a connective element which extends along the z-direction to the associated word line.

The global digit lines in the array patches 252g-257g are coupled to sense amplifiers in sense amplifier portions 222g-228g. The global digit lines in the array patches 252g and 256g are coupled to sense amplifier portions 222g and 224g, and the global digit lines in the array patches 253g and 257g are coupled to sense amplifier portions 226g and 228g. Each sense amplifier is coupled to two global digit lines, one in each of the two associated patches. The sense amplifiers in the first portion 222g are coupled to the first half of the global digit lines in patches 252g and 256g, the sense amplifiers in the second portion 224g are coupled to a second half of the global digit lines in patches 252g and 256g, the sense amplifiers in the portion 226g are coupled to a first half of the global digit lines in the patches 253g and 257g, and the sense amplifiers in the portion 228g are coupled to the second half of the global digit lines in the patches 253g and 257g. For example, if there are J global digit lines in each patch, then the portion 222g is coupled to GDL0 to GDL(J/2 – 1) while the portion 224g is coupled to GDL(J/2) to GDL(J-1).

The quilt also includes four row decoder and multiplexer driver regions 242g-248g. The row decoder and multiplexer driver regions 242g-248g include the global row decoder (e.g., 166 of FIG. 1) and the multiplexer drivers (e.g., 176 of FIG. 1). The regions 242g-248g are positioned above an associated one of the patches 252g-257g. Each region 242g-248g includes multiplexer drivers which are coupled to multiplexers in the associated patch 252g-257g. For example, each multiplexer driver may couple to a respective signal line extending in the x direction which couples to a row of multiplexers positioned at the intersection of local digit lines and global digit lines. The regions 242g and 246g are positioned such that a left edge of the regions 242g/246g are aligned above a left edge of the associated patches 252g/253g. Similarly, the regions 244g and 248g are positioned such that a right edge of the regions 244g/248g are positioned above a right edge of the associated patches 256g/257g.

In an example implementation, each of the memory patches 252g-257g may include about 8M bit of storage or about 8,192,000 memory cells. An example memory patch 252ghas 8000 word lines arranged in a grid in the yz plane of 80 word lines in the y direction and 100 word lines in the z direction. Each associated sense amplifier region 222g and 224g includes about 512 sense amplifiers coupled to half of the global digit lines which cross the array patch 252g, for a total of 1024 global digit lines in the memory patch 252g. The SWD region 272g includes 8000 SWDs, one for each word line, arranged in a grid in the xy plane of 80 SWDs in the y direction and 100 in the x direction. The multiplexer regions 242g-248g each include 80 multiplexer drivers, each coupled to a signal line coupled to 1024 multiplexers.

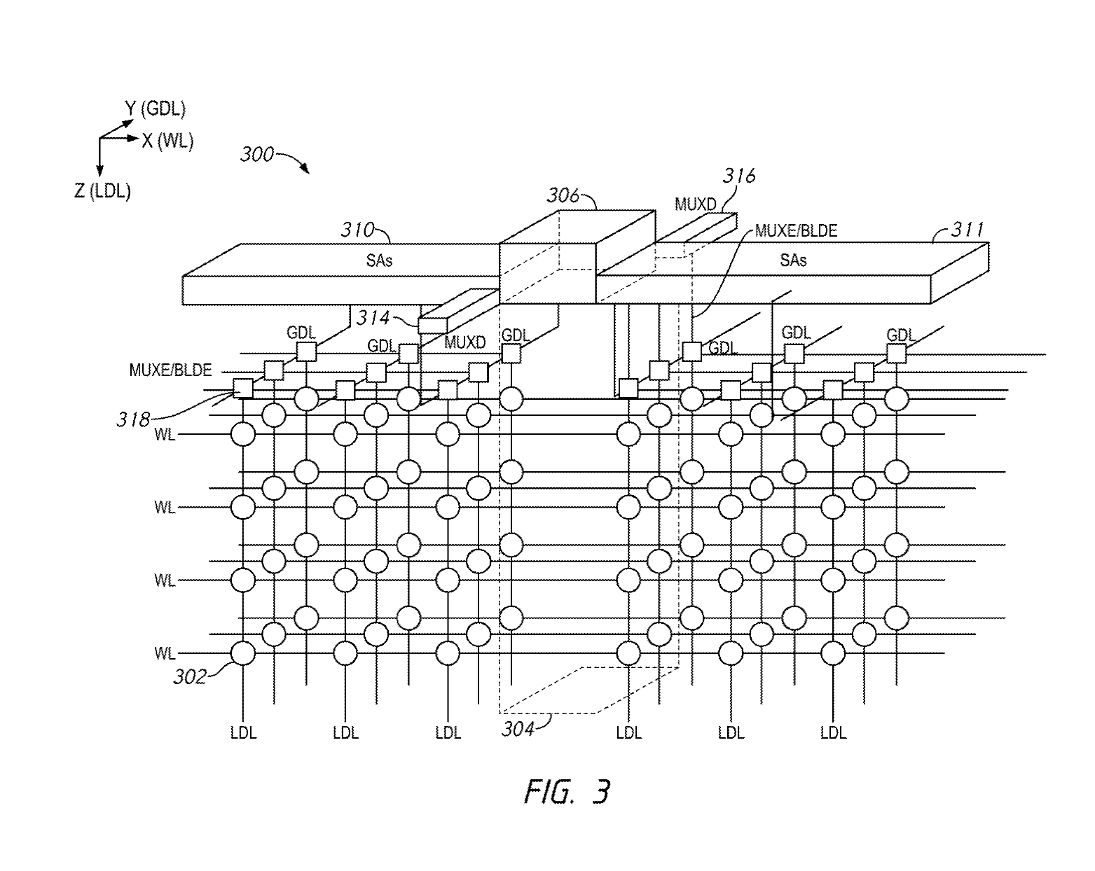

FIG. 3 is a perspective schematic diagram of a portion of a 3D memory array according to some embodiments of the present disclosure. The 3D memory array 300 represents a simplified view of an example portion of a memory array. For example, the 3D memory array 300 may represent a portion of the 3D array 180 of FIG. 1, and/or a representation of one or more of the quilt layouts 250b-g of FIGS. 2B-2G. The perspective of FIG. 3 shows an example set of memory cells 302 and their respective word lines, local digit lines and paired global digit lines. The view of FIG. 3 may be a simplified representational view which shows a relatively small number of word lines, global digit lines, local digit lines etc.

For the sake of illustration, FIG. 3 is generally illustrated with a similar layout to the layout 250b of FIG. 2B. However, the other example layouts of FIGS. 2C-2G may have 3D structures which are generally analogous to FIG. 3, but with different arrangements of components in the CMOS die and different connections between the array die and CMOS die. For example, FIG. 3 shows an example embodiment where a folded digit line structure is used where the sense amplifiers are coupled to a global digit line pair in the same memory patch. However, other example embodiments may use an open digit line structure where the sense amplifiers are coupled to global digit lines in two different memory patches.

The memory array 300 shows memory cells 302. Each memory cell 302 is positioned at the intersection of a word line WL and a local digit line LDL. Each LDL is associated with one or the other of a pair of global digit lines GDLs. The paired global digit lines are both coupled to a respective sense amplifier in the sense amplifier portions 310-311 (e.g., 174 of FIG. 1 and/or 222b-224b of FIG. 2B). The word lines are each coupled to a respective SWD in a SWD region 306 (e.g., 172 of FIG. 1 and/or 272b of FIG. 2B). The word lines are coupled via a staircase region 304 (e.g., 182 of FIG. 1, 282b of FIG. 2B) to the SWD region 306. Also shown are multiplexer driver regions 314 and 316 (e.g., 176 of FIG. 1 and/or 242b-248b of FIG. 2B) which couple to multiplexer circuits 318 (e.g., 184 of FIG. 1).

In some embodiments the SA regions 310-311, multiplexer drivers 314-316 and SWD region 306 may be in a different die than the die which includes the WL, LDL, GDL, memory cells 302 and multiplexer circuits 318. The view of FIG. 3 shows an example embodiment similar to the embodiment of FIG. 2B, where the sense amplifier regions 310-311 are positioned above the array patches. In particular, the view of FIG. 3 may represent the patches 252b and 254b of FIG. 2B, as well as their associated staircase region 282b, sense amplifier regions 222b and 224b, SWD regions 272b, and multiplexer driver regions 242b and 246b. The local column and row decoders are omitted from the view of FIG. 3. While specific reference is made to the components of FIG. 2B, many of the details of the components of FIG. 3 may also apply to the corresponding components of the layouts of FIGS. 2C-2G.

The staircase region 304 is a 3D region, which may be generally have the form of a rectangular prism. The staircase region 304 is positioned underneath the SWD region 306 in the z-direction. In some embodiments, the staircase region 304 may have the same x-y dimensions as the SWD region 306. The staircase region 304 is positioned between two sections of the word lines WL which intersect memory cells 302. However, the WLs may not intersect any memory cells while they pass through the staircase region 304. Vertical connection elements which extend in the z direction (not shown in FIG. 3) couple each word line to a respective SWD in the SWD region 306. A middle of the word lines WL may be positioned in the staircase region 304.

The word lines WL are arranged in a grid when considered in the yz plane. Similarly, the LDLs may be arranged in a grid when considered in the xy plane. The GDLs are generally arranged in a plane side-by-side with each other.

The LDLs are selectively coupled to one of the GDLs of the associated pair of GDLs through a multiplexer circuit 318. Some of the LDLs along the length of the GDL pair are coupled to one of the GDLs in the pair and some of the LDLs are coupled to the other. For example, each GDL in the pair of GDLs may be coupled to roughly half of the LDLs along the length of the GDL pair. The multiplexers are coupled to multiplexer drivers in a multiplexer driver region 314 or 316. A line of multiplexer drivers may be coupled in common by a multiplexer driver line along the x direction to a multiplexer driver in the multiplexer driver region 314/316. The multiplexer driver provides a multiplexer enable signal MUXE. When MUXE is active, all the multiplexers 318 which are coupled in common to that signal line will couple their respective LDL to the associated GDL.

In some embodiments, the multiplexer drivers 314/316 may also provide a bleed enable signal BLDE. When the bleed enable signal BLDE is active, the multiplexer couples the respective LDL to a ground voltage. In an example operation, the multiplexer driver associated with the row address provides an active MUXE signal and an inactive BLDE signal while the multiplexer drivers which are not associated with the row address provide an active BLDE signal and an inactive MUXE signal. Accordingly, during the operation, multiplexers which receive an active MUXE couple their LDLs to the GDL, while the other multiplexers along each GDL isolate their LDLs from the GDL and instead couple them to a ground voltage so they do not float.

FIG. 4 is a perspective view of a portion of a 3D memory device according to some embodiments of the present disclosure. The 3D memory device 400 may represent a portion of a memory device such as 100 of FIG. 1, 200a of FIG. 2A, 200c of FIG. 2C and/or 200d of FIG. 2D. The memory device 400 may include a memory array similar to the layout of the memory array 300 of FIG. 3 in some embodiments. The memory device 400 shows two adjacent sections of a memory array, similar to the view of the insets 250a and 250b of FIGS. 2A-2B. However, FIG. 4 shows a perspective view rather than a ‘top down’ view of the xy plane.

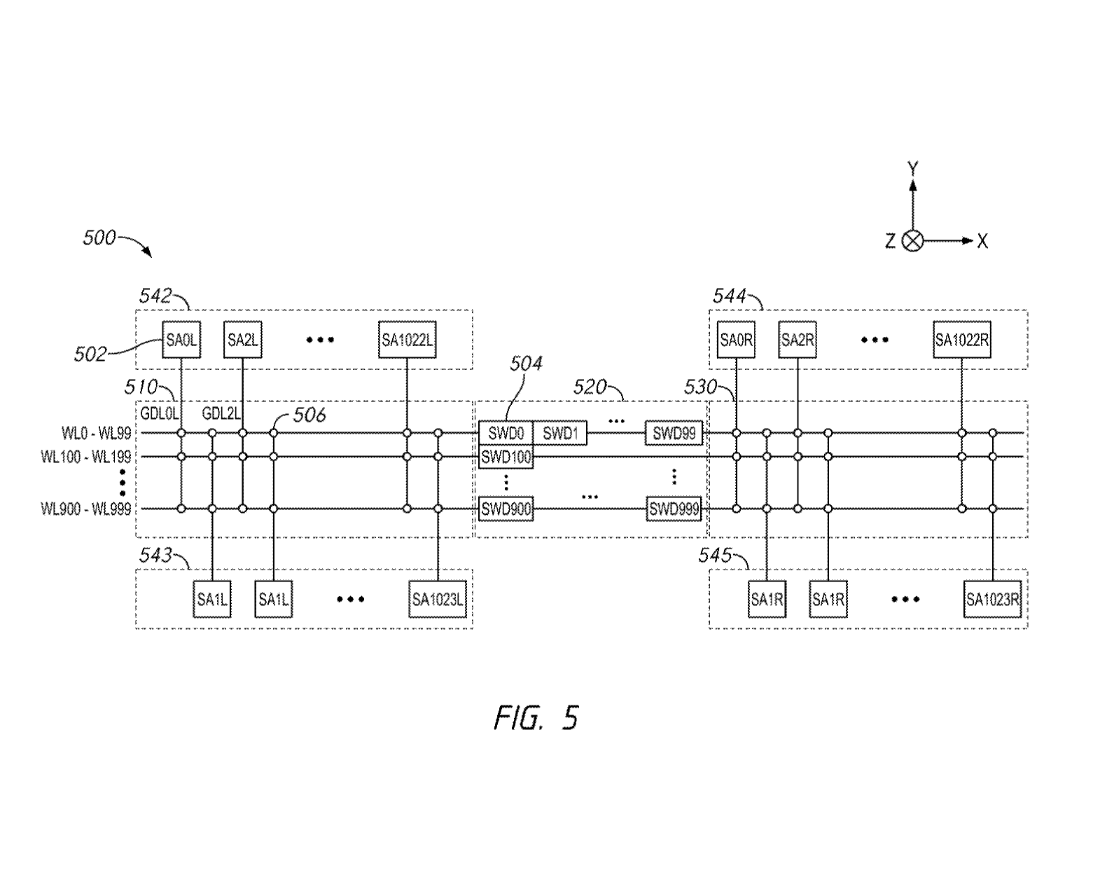

FIG. 4 shows planes 500, 600, and 700 which represent the views of FIGS. 5, 6, and 7 respectively. The plane 500 is a slice along an xy plane of the memory device 400 which intersects a first patch 402 (e.g., 252 of FIGS. 2A-2B), a SWD and staircase region 404 (e.g., 272 and 282 of FIGS. 2A-2B), a second memory patch 406 (e.g., 253 of FIGS. 2A-2B), and the sense amplifier regions 410-416 (e.g., 262-267 of FIGS. 2A-2B). The plane 600 is a slice along an xz plane of the memory which intersects a first memory patch 402, a SWD and staircase region 404 and a second memory patch 406. The plane 700 is a slice along a yz plane which intersects the first memory patch 402, a sense amplifier region 410 (e.g., 264 of FIGS. 2A-2B) and a third memory patch 408 (e.g., 256 of FIGS. 2A-2B). The first memory patch 402 and the second memory patch 406 are on opposites sides of the SWD and staircase region 404 and are part of a same memory section (e.g., 202a1 of FIG. 2A and/or 202b1 of FIG. 2B). The first memory patch 402 and the third memory patch 408 are on opposite sides of the sense amplifier region 410 and are part of different memory sections.

FIGS. 5-7 show different cross-sectional views of the 3D memory device 400 of FIG. 4. Each of FIGS. 5-7 is illustrated with respect to an example embodiment where each memory patch such as 402, 406 or 408 includes 1 Mbit of memory cells. Specifically, they are shown to include a grid of 10 word lines in the y direction and 100 word lines in the z direction, a grid of 10 LDLs in the y direction and 1024 LDLs in the x direction, and 1024 global digit lines side-by-side. This arrangement is shown as an illustrative example only. Other numbers and/or arrangements of word lines, global digit lines, local digit lines, memory cells, sense amplifiers, and so forth may be used in other example embodiments.

FIG. 5 is a cross sectional view of a 3D memory device according to some embodiments of the present disclosure. The cross section 500 shows the plane 500 of FIG. 4. The cross section 500 represents a view along an example xy plane, showing a top layer of word lines extending horizontally in the x direction, global digit lines extending in the y direction, and local digit lines 506 extending into the plane of the page in the z direction. The view of FIG. 5 shows a slice of the ‘top’ word lines in a stack of word lines, with additional word line extending down in the z direction. The cross sectional view of FIG. 5 shows a view of components which are not contained within a single plane. For example, the SWDs 504 and sense amplifiers 502 may generally be located in a plane which is above the plane of the word line in the z direction. However, they are shown in FIG. 5 to help aid in understanding the connections between and placement of various components.

The cross section 500 shows a first memory patch 510 (e.g., 252 of FIGS. 2A-2B and/or 402 of FIG. 4), a SWD and staircase region 520 (e.g., 272 of FIGS. 2A-2B and/or 404 of FIG. 4), and a second memory patch (e.g., 253 of FIGS. 2A-2B and/or 406 of FIG. 4). Also shown are sense amplifier regions 542-545 (e.g., 262-265 of FIGS. 2A-2B and/or 410-416 of FIG. 4). The view of FIG. 5 may represent a single memory section (e.g., 202a of FIG. 2A and/or 202b of FIG. 2B). The first memory patch 510 and the second memory patch 530 are located on opposite sides of the SWD and staircase region 520 in the x direction. The sense amplifier regions 542 and 543 are located on opposite sides of the first patch 510 in the y direction and the sense amplifier regions 544 and 545 are located on opposite sides of the second patch 530 in the y direction.

The cross section 500 includes word lines WL0 to WL999. The word lines are arranged in 10 stacks of 100 word lines each. So the top row of word lines includes the visible word line (WL0) as well as 99 more word lines extending in the z direction into the plane from the point of view of the drawing. The tops of local digit lines 506 are shown where they intersect a global digit lines GDL. The LDLs 506 are arranged in a grid layout in an xy plane, with a row of the grid including 1024 LDLs along the x direction and a column of the grid including of 10 LDLs along the y direction. The GDL extends in the y direction to a sense amplifier 502 in a sense amplifier region 542-545. The word lines WL extend from the first patch 510 to the second patch 530 under the SWD and staircase region 520.

The SWD and staircase region 520 includes a number of SWDs 504. There is a SWD for each word line. In this example there are 1000 SWD, arranged in a grid of 10x100 SWDs in the yz plane. The SWDs 504 are arranged in 10 rows, with each row over a stack of WLs. Since the stacks of WLs are arranged with 100 WLs in each stack, each row of SWDs has 100 SWDs. Each SWD along a row has a conductive element running in the z direction down to the WL it is coupled to. The SWDs along a ‘row’ of the grid of SWDs are coupled to conductive elements of different lengths, since they are coupled to different depths (in the z direction) of word line. When activated by a row activation command, the SWD associated with the row address activates the associated word line.

The global digit lines in each patch 510 and 530 are alternately coupled to one of the two adjacent sense amplifier regions. For example, the regions 542 and 544 may be even sense amplifier regions with SAs 502 coupled to even ones of the GDLs, while the region 543 and 545 may be odd sense amplifier regions with SAs 502 coupled to odd ones of the GDLs. In the example implementation of FIG. 5, each of the regions 542-545 includes 512 sense amplifiers 502, each coupled to a respective GDL. The global digit lines are labelled as left GDLs GDL0L to GDL1023L and right GDLs GDL0R to GDL1023R. Similarly, the sense amplifiers are labelled SA0L to SA1023L and SA0R to SA1023R. Except for edge sections, the sense amplifiers 502 may also generally be coupled to another GDL extending into an adjacent memory patch, not shown in FIG. 5.

When a row activation command is received along with a row address, the SWD 504 associated with that address activates the associated word line. When activated, the memory cells along that word line are coupled to the intersecting LDL 506, and change a voltage of the LDL and the GDL that LDL is coupled to based on the stored charge. The SA coupled to that GDL senses this change and amplifies it during a read operation, or drives a new value onto the GDL in a write operation.

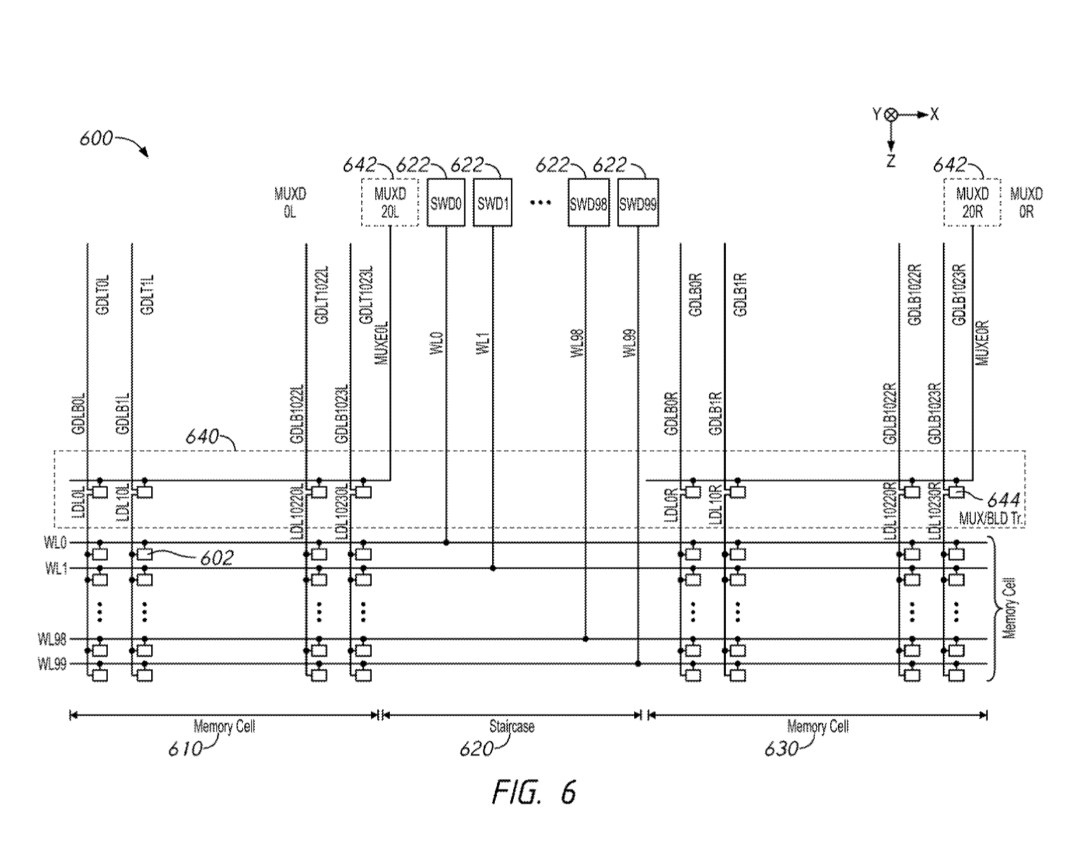

FIG. 6 is a cross sectional view of a 3D memory device according to some embodiments of the present disclosure. The cross section 600 shows the plane 600 of FIG. 4. The cross section 600 represents a view along an example xz plane, showing word lines running horizontally along the x direction, local digit lines running vertically along the z direction, and global digit lines running into and out of the plane of the page along the y direction. A portion of the global digit lines is also shown running vertically in the z direction to show the global digit lines coupling to their respective sense amplifiers. These vertical elements of the GDLs may alternate between being ‘in front’ of the plane of the cross section 600 or ‘behind’ the plane of the cross section 600 in the y direction. These may run to alternate sense amplifier regions such as 262/264 of FIGS. 2A-2B, 412/410 of FIG. 4, and/or 542/543 of FIG. 5.

The cross section 600 shows a first memory patch 610 (e.g., 252 of FIGS. 2A-2B, 402 of FIG. 4, and/or 510 of FIG. 5), a staircase region 620 (e.g., 272 of FIGS. 2A-2B, 404 of FIG. 4, and/or 520 of FIG. 5), and a second memory patch 630 (e.g., 253 of FIGS. 2A-2B, 406 of FIG. 4, and/or 530 of FIG. 5). The cross section 600 also shows a row a SWDs 622 associated with the WLs.

Each word line is coupled to a respective SWD 622. For example the cross section 600 shows SWD0 to SWD99, which are associated with WL0 to WL99 respectively. The SWDs 622 are located above in the z direction a staircase region 620 which is between the two patches 610 and 630 in the x direction. Each SWD 622 is coupled to a vertical conductive element which extends in the z direction to the associated word line. Along a row of SWDs 622 like the one shown in FIG. 6, each of these vertical conductive elements 624 may be a different length, since the WLs are at different depths in the z direction. In the example layout of FIG. 6, the shortest vertical conductive element 624, coupled to the ‘top’ word line WL0, is on the far left, while the longest vertical conductive element 624, coupled to the ‘bottom’ word line WL99, is on the far right. Other arrangements may be used in other example embodiments.

The cross section 600 shows a ‘stack’ of word lines. In this case the word lines WL0 to WL99. A number of LDLs extend vertically in the z direction and memory cells 602 are coupled at the intersection of the LDLs and the WLs. In the example implementation, there are 10,240 LDLs per patch from LDL0 to LDL10239. Each LDL seen in the cross section 600 represents a ‘top’ of a stack of 10 LDLs which extend in the y direction. Thus the leftmost LDL in the patch 610 is LDL0L, the next LDL is LDL10L, the next is LDL20L and so forth up to LDL10230L. Similarly, the LDLs in the patch 630 are LDL0R up to LDL10230R.

Responsive to a row activation command and a row address, the SWD 622 specified by the row address activates the coupled word line along the respective vertical conductive element 624. This couples the memory cells 602 in the two patches 610 and 630 to be coupled to the LDLs which intersect that word line. Those LDLs are in turn coupled to respective GDLs, which run to sense amplifier regions.

The view of FIG. 6 includes optional digit line multiplexers 644 (e.g., 184 of FIG. 1) in a digit line multiplexer region 640, and example multiplexer drivers 642. In some embodiments, these components may be omitted, and the LDLs may couple directly to the GDLs. In example embodiments where multiplexers are used, the multiplexers 644 selectively couple a set of LDLs to the respective GDLs. In the xz slice shown in FIG. 6, the multiplexers 644 of the left patch 610 are coupled to a multiplexer driver 642 and the multiplexers 644 of the right patch 630 are coupled to a multiplexer driver 642. Responsive to a signal from a global row decoder (e.g., 166 of FIG. 1), the multiplexer drivers 642 activate the multiplexers 644 to couple the LDLs along the cross section 600 to their respective GDLs. The other LDLs, which are stacked in the Y direction and not visible in FIG. 6, are coupled to multiplexers which are coupled to different multiplexer drivers, and thus are not coupled to the GDLs. This may help reduce the capacitance of the GDL since each GDL is only coupled to one LDL at a time.

In some embodiments, the SWDs 622 and, if used, multiplexer drivers 642 may be located in a CMOS die (e.g., 212 of FIG. 2B) while the memory cells, LDLs, WLs, and, if used multiplexer region 640 are located in an array die (e.g., 216 of FIG. 2B). The vertical conductive elements 624, as well as the vertical portions of the GDLs may extend from the array die to the CMOS die and may include contacts between the two dice not shown in FIG. 6.

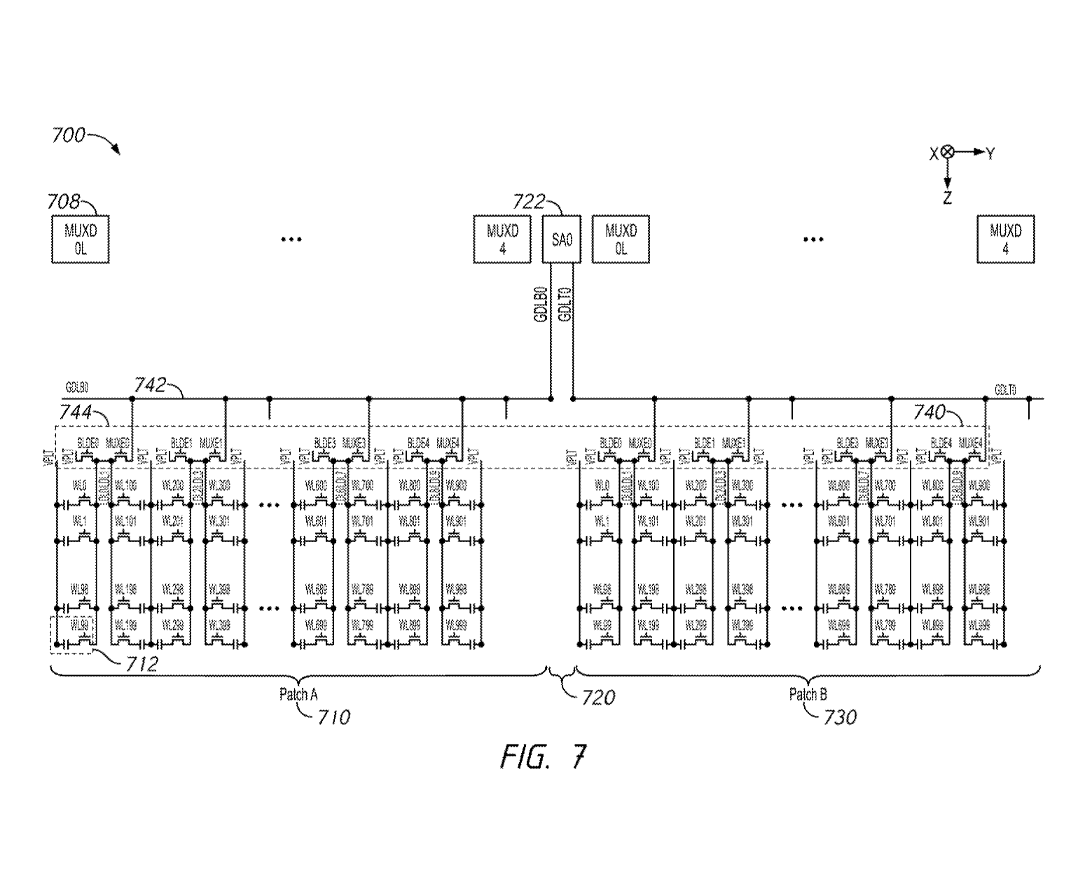

FIG. 7 is a cross sectional view of a 3D memory device according to some embodiments of the present disclosure. The cross section 700 shows the plane 700 of FIG. 4. The cross section 700 represents a view along an example yz plane, showing two global digit lines running horizontally in the y direction, local digit lines running vertically in the z direction, and word lines running through the plane of the page in the x direction. The cross section 700 shows a first memory patch 710 (e.g., 252 of FIGS. 2A-2B, 402 of FIG. 4, 510 of FIG. 5, and/or 610 of FIG. 6), a sense amplifier region 720 (e.g., 264 of FIGS. 2A-2B, 410 of FIG. 4, and/or 543 of FIG. 5), and a second memory patch 730 (e.g., 256 of FIGS. 2A-2B, and/or 408 of FIG. 4).

The cross section 700 intersects 1000 word lines in each memory patch 710 and 730. The word lines are organized in a grid in the yz plane with ten columns in the y direction that have 100 word lines each in the z direction. Accordingly, the cross section shows 1000 memory cells 712 organized in a grid of 10 memory cells in the x direction and 100 memory cells in the z direction in each of the two patches 710 and 730. Each column of memory cells 712 is coupled to a local digit line, here labelled LDL0 to LDL9. In the embodiment of FIG. 7, the LDLs are paired and each pair LDLs is coupled in common to a multiplexer transistor 742 and a bleed transistor 744 as described in more detail herein. In some embodiments, each LDL may couple separately through a multiplexer to the GDL, rather than being paired. In some embodiments, where multiplexers are not used, the LDLs may couple directly to the GDL.

Each memory cell 712 includes a capacitive element and a transistor. The gate of the transistor is coupled to the word line. One side of the capacitive element is coupled to a plate voltage VPLT, and the other side is coupled through the transistor to the LDL. When the word line is activated, the SWD (e.g., 504 of FIG. 5 and/or 622 of FIG. 6) drives a voltage along the word line which activates the transistor, coupling the capacitive element to the LDL.

The sense amplifier region 720 includes an example sense amplifier 722. The sense amplifier 722 is labelled as SA0 because it is coupled to GDL0 in two adjacent patches 710 and 730. To distinguish them, those GDLs are labelled as GDLB0 in the first patch 710 and GDLT0 in the second patch 730. During an example operation, one of the two GDLs is used to carry information, and the other is used as a reference. For example, if a word line is activated in the first patch 710, then information is carried along GDLB0 and GDLT0 is used as a reference. During a read operation, the sense amplifier 722 senses a difference between the GDL with information and the reference and then amplifies that difference.

FIG. 7 shows an optional multiplexer region 740 along with optional multiplexer drivers 708 which operates the multiplexer transistors 742 and bleed transistors 744 of the multiplexer region 740. Each pair of LDLs is coupled to the GDL through a multiplexer transistor 742. The gate of the multiplexer transistor 742 is coupled to a multiplexer control signal MUXE which is coupled to respective multiplexer drivers 708 along a signal line which runs in the x direction. Also shown in FIG. 7 is an optional bleed transistor 744 which couples each pair of LDL to the plate voltage VPLT. The bleed transistors 744 have gates coupled to bleed control signals BLDE which are coupled to respective multiplexer drivers 708 along signal lines which run in the x direction. The multiplexer drivers 708 are controlled by global row decoders (e.g., 166 of FIG. 1).

During an example operation, if a row address is received associated with the first patch 710, then the multiplexer driver 708 which controls the LDL which intersects the WL activates the multiplexer transistor 742 by providing MUXE, and each of the other multiplexer drivers 708 provide BLDE to their respective bleed transistor 744 to couple those LDLs to VPLT and prevent them from floating. For example, if the row address indicates WL 101, which intersects LDL1, then MUXD0 provides MUXE0, which couples both LDL0 and LDL1 to GDLB0. The multiplexer drivers MUX1 to MUX4 provide BLDE1 to BLDE4 respectively, which couple the other LDLs LDL2 to LDL9 to VPLT through the respective bleed transistors 744.

FIG. 8 is a perspective view of a portion of a memory device according to some embodiments of the present disclosure. The memory device 800 may represent a portion of a memory device such as 100 of FIG. 1, 200a of FIG. 2A and/or 200F of FIG. 2F. The memory device 800 may include a memory array similar to the layout of the memory array 300 of FIG. 3 in some embodiments. The memory device 800 shows two adjacent sections of a memory array, similar to the view of the insets 250F of FIGS. 2F. However, FIG. 8 shows a perspective view rather than a ‘top down’ view of the xy plane.

The memory device 800 includes a first die 840 (e.g., 212a of FIG. 2A) and a second die 850 (e.g., 216a of FIG. 2A). The first die 840 may includes the sense amplifiers, SWDs, and multiplexer drivers. For example the first die 840 may be a CMOS die. The second die 850 includes the memory array patches.

The memory device 800 includes a first section which includes a first memory patch 802, a second memory patch 806 and a SWD region 804 in between. The memory device also includes a second section which includes a third memory patch 812, a fourth memory patch 816 and a second SWD region 814 in between. An interpatch region 810 is between the patches 802 and 812 and the patches 806 and 816. In the perspective view of FIG. 8, spacer regions are shown on the surface of the first die 840 which are above the memory patches in the array die 850 below.

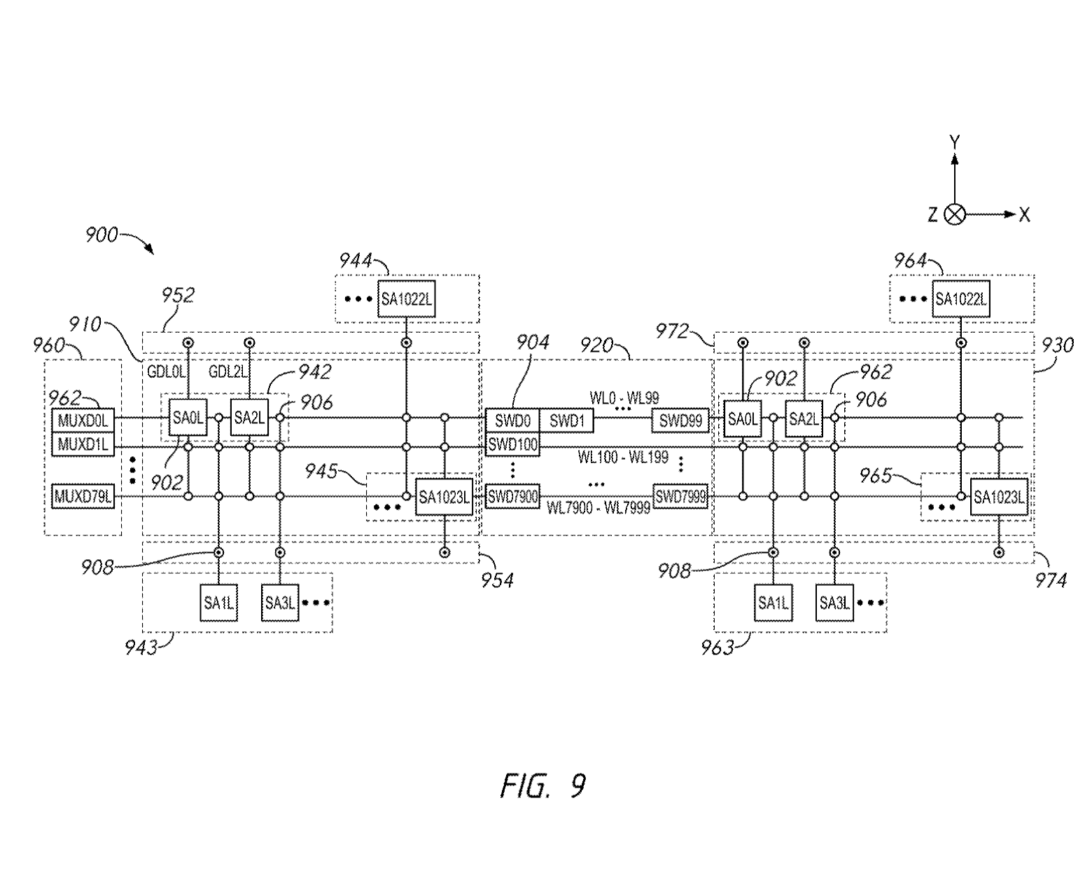

FIG. 8 shows planes 900, 1000, and 1100 which represent the cross-sectional views of FIGS. 9, 10, and 11 respectively. The plane 900 is a slice along an xy plane of the memory 800 which intersects a first patch 802 (e.g., 252f of FIG. 2F), a SWD region 804 (e.g., 272f and 282f of FIG. 2F), a second memory patch 806 (e.g., 253f of FIG. 2F), and the sense amplifier regions 820-825 (e.g., 262f-267f of FIG. 2F). Also shown in the view of the plane 900 are the interpatch region 810 as well as additional regions associated with the two patches 802 and 806 such as the SA regions 826 and 828 and an additional interpatch region and additional SA regions not shown in FIG. 8 which are above the patches 802 and 806 in the y direction.

The plane 1000 is a slice along an xz plane of the memory which intersects a second memory patch 812, an SWD and staircase region 814 and a second memory patch 816. The plane 1000 is a slice along a yz plane which intersects the first memory patch 802, interpatch region 810, and the third memory patch 812. The view 1000 also intersects sense amplifier regions 826 and 822 which are above the patches 812 and 802 respectively.

FIGS. 9-11 show different cross-sectional views of the memory device 800 of FIG. 8. Each of FIGS. 9-11 is illustrated with respect to an example embodiment where each memory patch such as 802, 806 or 808 includes 8 Mbit of memory cells. Specifically, they are shown to include a grid of 80 word lines in the y direction and 100 word lines in the z direction, a grid of 80 LDLs in the y direction and 1024 LDLs in the x direction, and 1024 global digit lines side-by-side. This arrangement is shown as an illustrative example only. Other numbers and/or arrangements of word lines, global digit lines, local digit lines, memory cells, sense amplifiers, and so forth may be used in other example embodiments.

FIG. 9 is a cross sectional view of a memory device according to some embodiments of the present disclosure. The cross section 900 shows the plane 900 of FIG. 8. The cross section 900 represents a view along an example xy plane, showing a top layer of word lines extending horizontally in an x direction, global digit lines extending in a y direction, and local digit lines extending into the plane of the page in the z direction from the multiplexer circuits 906 where they intersect the GDL. The view of FIG. 9 shows a slice of the ‘top’ word lines in a stack of word lines, with additional word line extending down in the z direction. The cross sectional view of FIG. 9 shows a view of components which are not contained within a single plane. For example, the SWDs 904 and sense amplifiers 902 may generally be located in a plane which is above the plane of the word line in the z direction. Certain components in the plane of the array, such as certain multiplexers 906 may be occluded by the sense amplifiers which are above the memory patch.

The cross section 900 shows a first memory patch 910 (e.g., 252f of FIG. 2F and/or 802 of FIG. 8), a SWD and staircase region 920 (e.g., 272f of FIG. 2F and/or 804 of FIG. 8), and a second memory patch 930 (e.g., 253f of FIG. 2F and/or 806 of FIG. 8). Also shown are sense amplifier regions 942-949 (e.g., 222f-238f of FIG. 2F and/or 308-311 of FIG. 3). The view of FIG. 9 may represent a single memory section (e.g., 202a of FIG. 2A). In particular, the view of FIG. 9 may be similar to the layout of FIG. 2F, where the sense amplifier regions are positioned over the array. The first memory patch 910 and the second memory patch 920 are located on opposite sides of the SWD and staircase region 920 in the x direction.

The sense amplifier portions 942-945 are associated with the first patch 910. The sense amplifier portions 962-965 are associated with the second patch 920. The portions 942 and 944 form a first region and are associated with even GDLs. The portions 943 and 945 form a second region and are associated with odd GDLs. Similarly, the portions 962/964 couple to even GDLs and the portions 963/965 couple to even GDLs. The portions 942, 945, 962, and 965 are located above the memory patches 910 and 930 respectively. The portions 943, 946, 963, and 966 are located above patches which are adjacent (in the y direction) from the patches 910 and 930.

Interpatch regions 952, 954, 972 and 974 form a border between the array patches 910 and 920 and adjacent patches in the y directions, not shown in FIG. 9. For example, the interpatch region 952 is between the patch 910 and another patch which is above it in the +y direction and the interpatch region 954 is between the patch 910 and another patch which is below it in the -y direction. Within the interpatch region, vertical elements 908 couple the GDLs from the die where the array is located to the die where the SAs 902 are located.

The cross section 900 includes word lines WL0 to WL7999. The word lines are arranged in 80 stacks of 100 word lines each. So the top row of word lines includes the visible word line (WL0) as well as 99 more word lines extending in the z direction into the plane from the point of view of the drawing. Multiplexer circuits couple a ‘top’ of one or more LDLs to associated GDLs. The LDLs are arranged in a grid layout in an xy plane, with a row of the grid including 1024 LDLs along the x direction and a column of the grid including of 80 LDLs along the y direction. The word lines WL extend from the first patch 910 to the second patch 930 under the SWD and staircase region 920.

The SWD and staircase region 920 includes a number of SWDs 904. There is a SWD for each word line. In this example there are 8000 SWD, arranged in a grid of 80x100 SWDs in the yz plane. The SWDs 904 are arranged in 80 rows, with each row over a stack of WLs. Since the stacks of WLs are arranged with 100 WLs in each stack, each row of SWDs has 100 SWDs. Each SWD along a row has a conductive element running in the z direction down to the WL it is coupled to. The SWDs along a ‘row’ of the grid of SWDs are coupled to conductive elements of different lengths, since they are coupled to different depths (in the z direction) of word line. When activated by a row activation command, the SWD associated with the row address activates the associated word line.

The global digit lines in each patch 910 and 930 are alternately coupled to different sense amplifier portions. A first half of the GDLs in a patch are coupled either to a first portion or a second portion, and a second half of the GDLs in a patch are coupled either to a third portion or a fourth portion. For each half of the GDLs, either the even or odd GDLs are coupled to a portion which is over the patch or over an adjacent patch and whether it is the even or odds is reversed in the other half.

For example, in patch 910, the even GDLs from GDL0 to GDL510 are coupled to SA circuits 902 in the portion 942, while the odd GDLs from GDL1 to GDL511 are coupled to SA circuits 902 in the portion 943. Similarly, the even GDLs from GDL512 to GDL1022 are coupled to SA circuits 902 in the portion 944 while the odd GDLs from GDL513 to GDL1023 are coupled to SA circuits 902 in the portion 945. The even GDLs GLD0 to GDL1022 are coupled to sense amplifier circuits 902 through the first interpatch region 952, while the odd GDLs GDL1 to GDL1023 are coupled to sense amplifier circuits 902 through the second interpatch region 954.

The sense amplifier regions 942-945 and 962-965 are offset relative to the interpatch region they are associated. The sense amplifier portion 942 is below the interpatch region 952 in the y direction while the sense amplifier portion 944 is above the interpatch region 952 in the y direction. Similarly, the sense amplifier portion 943 is below the interpatch region 954 in the y direction while the sense amplifier portion 945 is above the interpatch region 954 in the y direction. Because of this the sense amplifiers are coupled to the vertical conductive elements 908 by horizontal elements which couple from a ‘top’ of the vertical conductive element 908 over to the sense amplifier.

The LDLs are coupled to the GDL by a multiplexer circuit 906. The multiplexer circuit is generally positioned between a ‘top’ of the LDL in the z direction and the GDL. In the embodiment of FIG. 5, there is a multiplexer circuit 906 for each LDL. Other example embodiments may group multiple LDLs together through each multiplexer circuit 906. A multiplexer driver circuit 962 is coupled by one or more signal lines extending in the x direction to a row of multiplexer driver circuits. The signal lines provide multiplexer enable signals, bleed enable signals or both. A row of multiplexers 906 may be coupled to a multiplexer driver in common.

In the embodiment of FIG. 5, the multiplexers 906 are arranged in an xy grid of 80 rows and 1024 columns. Each row of 1024 multiplexers is coupled in common to a multiplexer driver 962 in a multiplexer driver region 960. Accordingly, there are 80 multiplexer drivers 962. Responsive to a row address, one of the multiplexer drivers 962 may activate the multiplexers 906 along the associated row, while the other multiplexer drivers remain inactive (and/or provide the bleed signal at an active level). The active multiplexer driver is the one in the same row as the word line. For example, if one of the word lines WL0 to WL99 is activated, then the first multiplexer driver MUXD0 will be activated.

When a row activation command is received along with a row address, the SWD 904 associated with that address activates the associated word line. The associated multiplexer driver 962 activates the multiplexer circuit 906 so that the LDLs which intersect the active word line are coupled to the respective GDLs. When the word line is activated, the memory cells along that word line are coupled to the intersecting LDL 906, and change a voltage of the LDL and the GDL that LDL is coupled to based on the stored charge. The SA coupled to that GDL senses this change and amplifies it during a read operation, or drives a new value onto the GDL in a write operation.

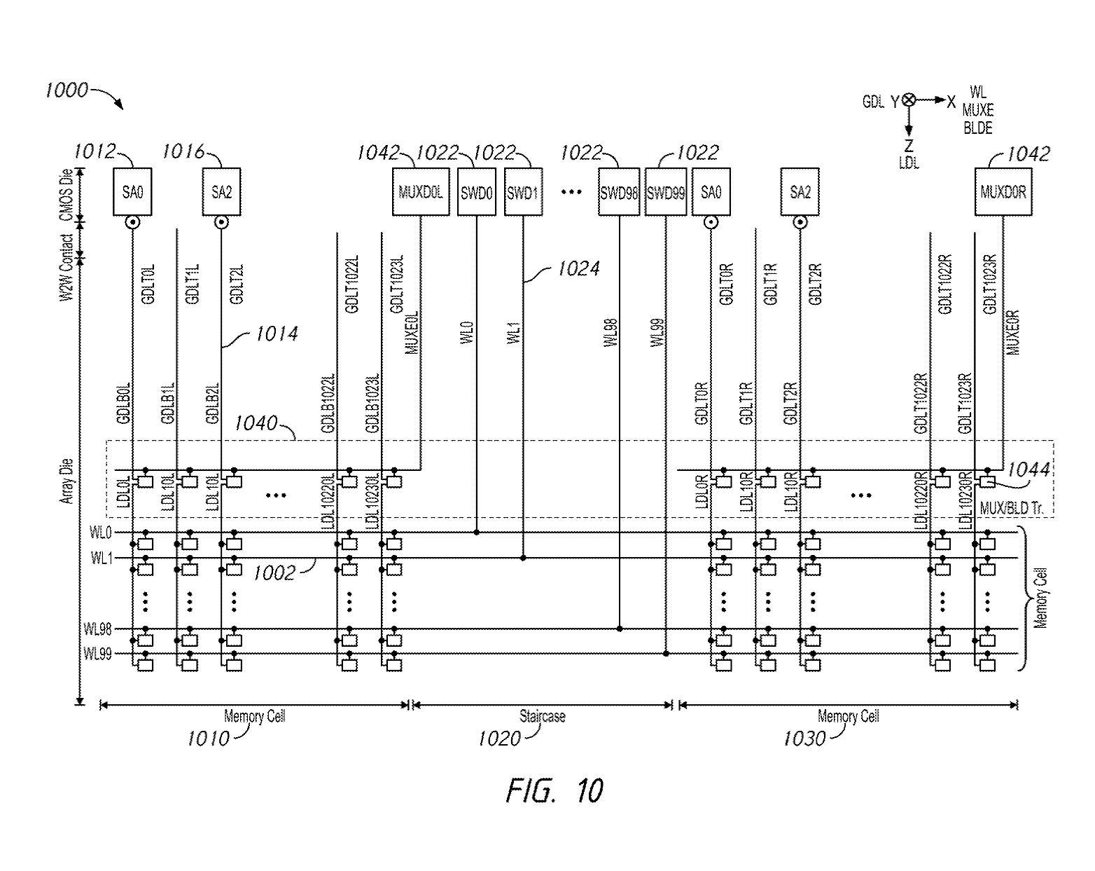

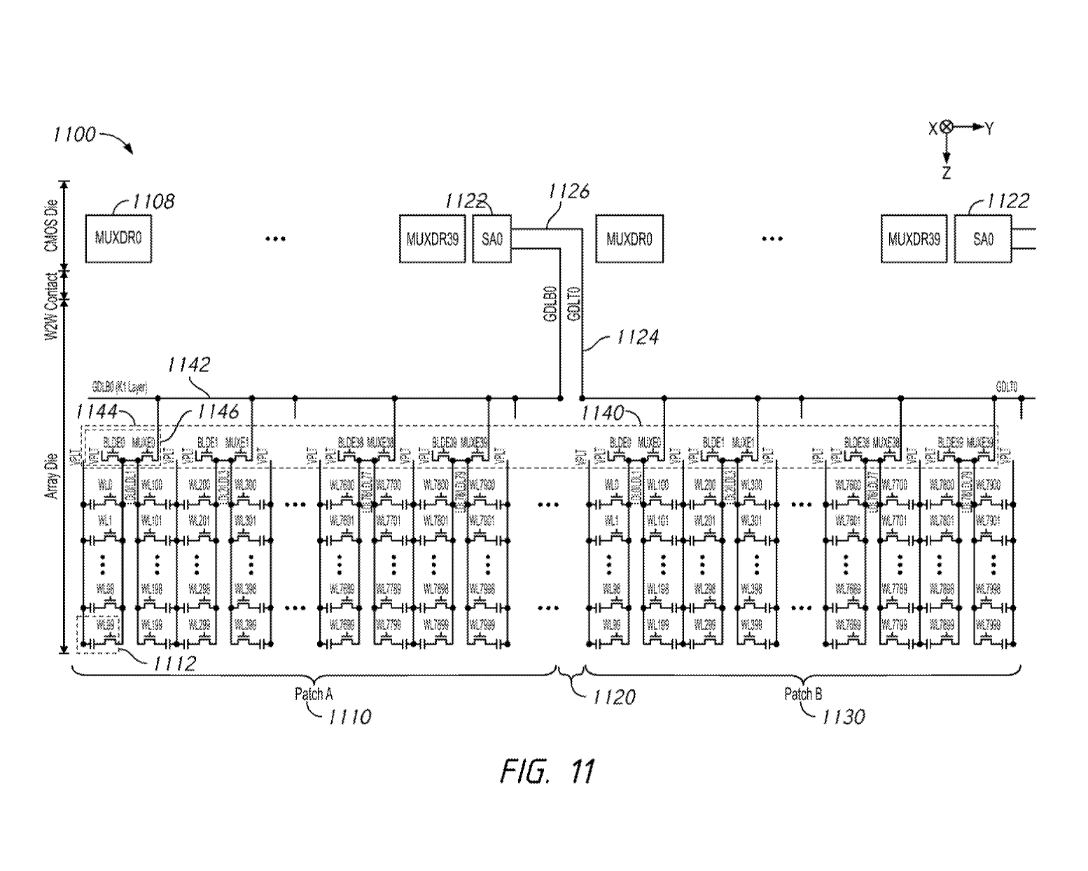

FIG. 10 is a cross sectional view of a memory device according to some embodiments of the present disclosure. The cross section 1000 shows the plane 1000 of FIG. 8. The cross section 1000 represents a view along an example xz plane, showing word lines running horizontally along the x direction, local digit lines running vertically along a z direction, and global digit lines running into and out of the plane of the page along a y direction. A vertical conductive element 1014 is shown in an interpatch region (e.g., 810 of FIG. 8) which runs ‘behind’ the plane of the cross section 1000. The vertical conductive element 1014 is coupled to a horizontal element 1016 which couples to the sense amplifiers 1012, which are positioned over the memory cells. The sense amplifiers 1012 shown are the ones in the portions which couple to even digit lines for example the ones in regions 826 and 828 of FIG. 8.

The cross section 1000 shows a first memory patch 1010 (e.g., 252f of FIG. 2F), 802 of FIG. 8 and/or 910 of FIG. 9) a staircase region 1020 (e.g., 272f of FIG. 2F), 804 of FIG. 8, and/or 920 of FIG. 7 and a second memory patch 1030 (e.g., 253f of FIG. 2F , 806 of FIG. 8, and/or 930 of FIG. 9). The cross section 1000 also shows a row a SWDs 1022 associated with the WLs.

Each word line is coupled to a respective SWD 1022. For example the cross section 1000 shows SWD0 to SWD99, which are associated with WL0 to WL99 respectively. The SWDs 1022 are located above in the z direction a staircase region 1020 which is between the two patches 1010 and 1030 in the x direction. Each SWD 1022 is coupled to a vertical conductive element 1024 which extends in the z direction to the associated word line. Along a row of SWDs 1022 like the one shown in FIG. 10, each of these vertical conductive elements 1024 may be a different length, since the WLs are at different depths in the z direction. In the example layout of FIG. 10, the shortest vertical conductive element, coupled to the ‘top’ word line WL0, is on the far left, while the longest vertical conductive element, coupled to the ‘bottom’ word line WL99, is on the far right. Other arrangements may be used in other example embodiments.

The cross section 1000 shows a ‘stack’ of word lines. In this case the word lines WL0 to WL99. A number of LDLs extend vertically in the z direction and memory cells 1002 are coupled at the intersection of the LDLs and the WLs. In the example implementation, there are 81,920 LDLs per patch from LDL0 to LDL81919. Each LDL seen in the cross section 1000 represents a ‘top’ of a stack of 80 LDLs which extend in the y direction. Thus the leftmost LDL in the patch 1010 is LDL0L, the next LDL is LDL80L, the next is LDL160L and so forth up to LDL81840L. Similarly, the LDLs in the patch 1020 are LDL0R up to LDL81919R.

The view of FIG. 10 includes digit line multiplexers 1044 (e.g., 184 of FIG. 1) in a digit line multiplexer region 1040, and example multiplexer drivers 1042. The multiplexers 1044 selectively couple a set of LDLs to the respective GDLs. In the xz slice shown in FIG. 10, the multiplexers 1044 of the left patch 1010 are coupled to a multiplexer driver 1042 and the multiplexers 1044 of the right patch 1030 are coupled to a multiplexer driver 1042. The multiplexer drivers 1042 are coupled along signal lines which extend in the x direction the multiplexer circuits 1044. For example a first signal line couples the multiplexers 1044 in the patch 1010 to the driver 1042 and a second signal line couples the multiplexers 1044 in the patch 1030 to the driver 1042. The multiplexer drivers 1042 and signal line represent a single row. Additional rows are stacked in the y direction in and out of the plane of the page.

Responsive to a row activation command and a row address, the SWD 1022 specified by the row address activates the coupled word line along the respective vertical conductive element 1024. This couples the memory cells 1002 in the two patches 1010 and 1030 to be coupled to the LDLs which intersect that word line. Responsive to a signal from a global row decoder (e.g., 166 of FIG. 1) based on the row address, the multiplexer drivers 1042 activate the multiplexers 1044 to couple the LDLs along the cross section 1000 to their respective GDLs. The memory cells along the activated WL drive a voltage onto the intersecting LDLs, which then drive a voltage to the coupled GDL through the activated multiplexers 1044. The other LDLs, which are stacked in the Y direction and not visible in FIG. 10, are coupled to multiplexers which are coupled to different multiplexer drivers, and thus are not coupled to the GDLs. This may help reduce the capacitance of the GDL since each GDL is only coupled to one LDL at a time.

In some embodiments, the SWDs 1022 and multiplexer drivers 1042 may be located in a CMOS die (e.g., 212 of FIG. 2B) while the memory cells, LDLs, WLs, and, if used multiplexer region 1040 are located in an array die (e.g., 216 of FIG. 2B). The vertical elements 1024, as well as the vertical portions of the GDLs may extend from the array die to the CMOS die and may include contacts between the two dice not shown in FIG. 10.

FIG. 11 is a cross sectional view of a memory device according to some embodiments of the present disclosure. The cross section 1100 shows the plane 1100 of FIG. 11. The cross section 1100 represents a view along an example yz plane, showing two global digit lines running horizontally in the y direction, local digit lines running vertically in the z direction, and word lines running through the plane of the page in the x direction. The cross section 1100 shows a first memory patch 1110 (e.g., 252f of FIG. 2F, 802 of FIG. 8, 910 of FIG. 9, and/or 1010 of FIG. 10), an interpatch region 1120 (e.g., 810 of FIG. 8), and a second memory patch 1130 (e.g., 256f of FIGS. 2F, and/or 808 of FIG. 8).

The cross section 1100 intersects 8000 word lines in each memory patch 1110 and 1130. The word lines are organized in a grid in the yz plane with eighty columns in the z direction that have 100 word lines each. Accordingly, the cross section shows 16,000 memory cells 1112, 8000 in each patch 1110 and 1130, with each patch organized in a grid of 80 memory cells in the x direction and 100 memory cells in the z direction. Each column of memory cells 1112 is coupled to a local digit line, here labelled LDL0 to LDL79. In the embodiment of FIG. 7, the LDLs are paired and each pair LDLs is coupled in common to a multiplexer circuit 1146 as described in more detail herein. In some embodiments, each LDL may couple separately through a multiplexer to the GDL, rather than being paired.

Each memory cell 1112 includes a capacitive element and a transistor. The gate of the transistor is coupled to the word line. One side of the capacitive element is coupled to a plate voltage VPLT, and the other side is coupled through the transistor to the LDL. When the word line is activated, the SWD drives a voltage along the word line which activates the transistor, coupling the capacitive element to the LDL.

The cross section 1100 shows a sense amplifier 1122. The sense amplifier 1122 is labelled as SA0 because it is coupled to GDL0 in two adjacent patches 1110 and 1130. To distinguish them, those GDLs are labelled as GDLB0 in the first patch 1110 and GDLT0 in the second patch 1130. During an example operation, one of the two GDLs is used to carry information, and the other is used as a reference. For example, if a word line is activated in the first patch 1110, then information is carried along GDLB0 and GDLT0 is used as a reference. During a read operation, the sense amplifier senses a difference between the GDL with information and the reference and then amplifies that difference. During a write operation, the sense amplifier drives one GDL to a voltage representing the write value and the other to the compliment of the write value.

The sense amplifier 1122 is shown positioned above the memory patch 1110. Vertical conductive elements 1124 in the interpatch region 1120 couple the GLDs GDLB0 and GDLT0 up from the array die to the CMOS die. Horizontal conductive elements 1126 couple the vertical conductive elements from the interpatch region 1120 to the location of the sense amplifier 1122 over the array patch 1110. Another sense amplifier is shown over the patch 1130 as an example, however that sense amplifier may be in a different plane offset in the x direction and is coupled to GDLT1 and a GDLB1 from an adjacent patch (in the +y direction) not shown in FIG. 11.

FIG. 7 shows multiplexer region 1140 along with the multiplexer drivers 1108 which operates the multiplexer region. The multiplexer circuits 1146 are implemented by a multiplexer transistor 1142 and an optional bleed transistor 1144. Each pair of LDLs is coupled to the GDL through a multiplexer transistor 1142. The gate of the multiplexer transistor 1142 is coupled to a multiplexer control signal MUXE which is coupled to respective multiplexer drivers 1108 along a signal line which runs in the x direction. The bleed transistor 1144 selectively couples each pair of LDL to the plate voltage VPLT. The bleed transistors 1144 have gates coupled to bleed control signals BLDE which are coupled to respective multiplexer drivers 1108 along signal lines which run in the x direction. The multiplexer drivers 1108 are controlled by global row decoders (e.g., 166 of FIG. 1).

During an example operation, if a row address is received associated with the first patch 1110, then the multiplexer driver 1108 which controls the LDL which intersects the WL activates the multiplexer transistor 1142 by providing MUXE, and each of the other multiplexer drivers 1108 provide BLDE to their respective bleed transistor 1144 to couple those LDLs to VPLT and prevent them from floating. For example, if the row address indicates WL 101, which intersects LDL1, then MUXD0 provides MUXE0, which couples both LDL0 and LDL1 to GDLB0. The multiplexer drivers MUX1 to MUX4 provide BLDE1 to BLDE4 respectively, which couple the other LDLs LDL2 to LDL9 to VPLT through the respective bleed transistors 1144.

In some embodiments, multiplexers may be activated on both sides of the sense amplifier in order to match capacitance between the signal and reference GDLs. Using the example above, a row address is received which indicates WL101 in patch 1110. Accordingly, the signal will be along GDLB0 and the reference will be GDLT0. The multiplexer drivers MUXDL0 and MUXDR0 provide the signals MUXE0 to both patches 1110 and 1130. This causes the LDLs LDL0 and LDL1 in both patches 1110 and 1130 to be coupled to GDLB0 and GDLT0 respectively. The multiplexer driver MUXDL0 and MUXDR0 provide BLDE0 at an inactive level. The other multiplexer drivers MUXDL1 to MUXDL39 and MUXDR1 to MUXDR39 provide MUXE1 to MUXE39 at an inactive level and BLDE1 to BLDE39 at an active level.

FIG. 12 is a perspective view of a memory quilt of a memory device according to some embodiments of the present disclosure. The memory quilt 1200 may represent a portion of a memory device such as 100 of FIG. 1 and/or 200a of FIG. 2a. In particular, the memory quilt 1200 represents an example implementation of the memory quilt layout 250g of FIG. 2G. The memory quilt 1200 may include one or more memory arrays which have layouts similar to the memory array 300 of FIG. 3 in some embodiments. The memory quilt 1200 may be generally similar to the quilt 250g of FIG. 2G. However, FIG. 12 shows a perspective view rather than a ‘top down’ view of the xy plane.

The memory device which includes the quilt 1200 includes a first die 1240 (e.g., 212g of FIG. 2G) and a second die 450g (e.g., 216g of FIG. 2G). The first die 1240 may includes the sense amplifiers, SWDs, and multiplexer drivers. For example, the first die 1240 may be a CMOS die. The second die 1250 includes the memory array patches and staircase regions.

The memory quilt 1200 includes a first memory patch 1202, a second memory patch 1206 and a SWD region 1204 in between. The memory quilt 1200 also includes a third memory patch 1212, a fourth memory patch 1216 and a second SWD region 1214 in between. An interpatch region 1210 is between the patches 1202 and 1212 and the patches 1206 and 1216. In the perspective view of FIG. 12, spacer regions are shown on the surface of the first die 1240 which are above the memory patches 1202-1216 in the array die 1250 below.

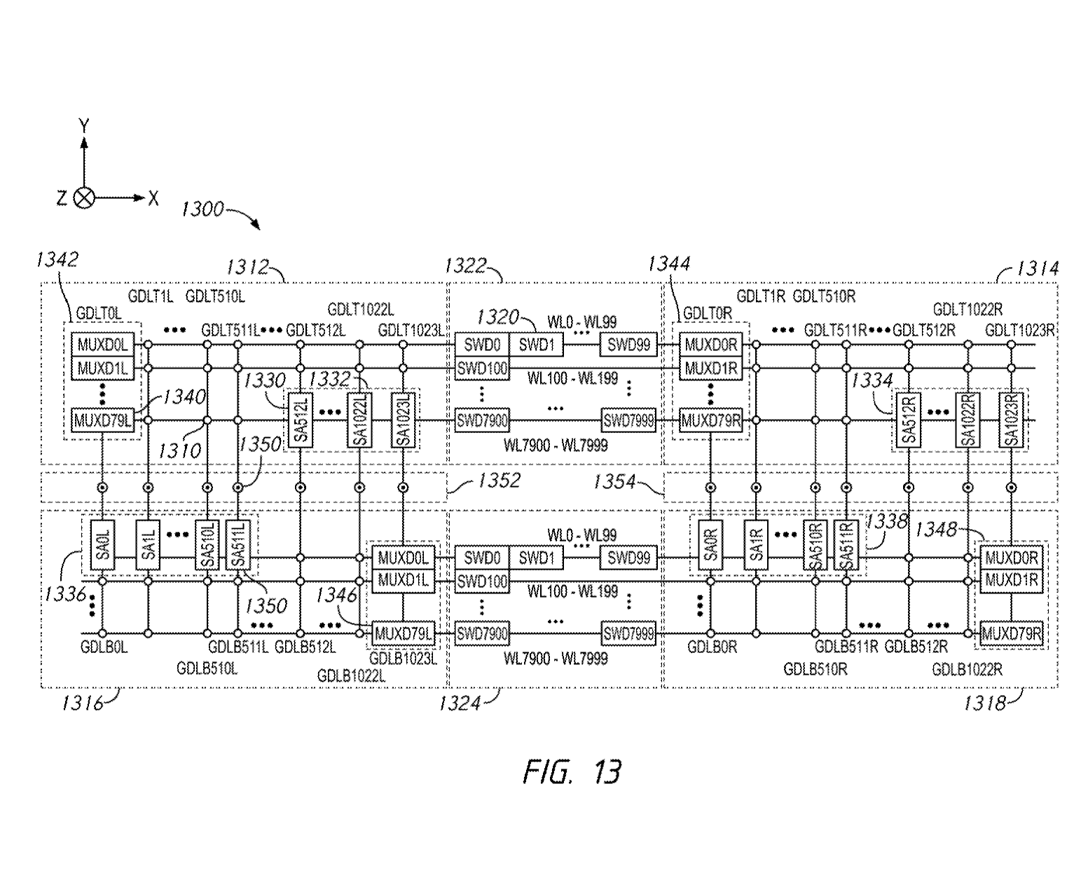

FIG. 4 shows planes 1300, 1400, and 1500 which represent the cross-sectional views of FIGS. 13, 14, and 15 respectively. The plane 1300 is a slice along an xy plane of the memory 1200 shows the memory patches 1202, 1206, 1212, and 1216 (e.g., 252g-257g of FIG. 2G), the SWD and staircase regions 1204 and 1214 (e.g., 272g/282g and 276g/286g of FIG. 2G), the multiplexer regions 1252, and the sense amplifier regions 1223-1226 (e.g., 222g-228g of FIG. 2G). Also shown in the view of the plane 1300 is the interpatch region 1210.

The plane 1400 is a slice along an xz plane of the memory which intersects a second memory patch 1212, an SWD and staircase region 1214 and a second memory patch 1216. The plane 1400 also intersects sense amplifier regions 1226 and 1228 and multiplexer driver regions 1252. The plane 1500 is a slice along a yz plane which intersects the first memory patch 1202, interpatch region 1210, and the third memory patch 1212. The view 1500 also intersects sense amplifier regions 1226 and 1222 which are above the patches 1212 and 1202 respectively.

FIGS. 13-15 show different cross-sectional views of the memory device 1200 of FIG. 12. Each of FIGS. 13-15 is illustrated with respect to an example embodiment where each memory patch such as 1202, 1206 or 1208 includes 8 Mbit of memory cells. Specifically, they are shown to include a grid of 80 word lines in the y direction and 100 word lines in the z direction, a grid of 80 LDLs in the y direction and 1024 LDLs in the x direction, and 1024 global digit lines side-by-side. This arrangement is shown as an illustrative example only. Other numbers and/or arrangements of word lines, global digit lines, local digit lines, memory cells, sense amplifiers, and so forth may be used in other example embodiments.

FIG. 13 is a top-down view of a memory quilt according to some embodiments of the present disclosure. The quilt 1300 shows a view of an example quilt such as 250g of FIG. 2G and/or 1200 of FIG. 12. The cross section 1300 shows the plane 1300 of FIG. 12. The quilt 1300 represents a view along an example xy plane, showing a top layer of word lines extending horizontally in an x direction, global digit lines extending in a y direction, and local digit lines extending into the plane of the page in the z direction from the multiplexer circuits 1310 where they intersect the GDL. The view of FIG. 13 shows a slice of the ‘top’ word lines in a stack of word lines, with additional word line extending down in layers in the z direction. The ‘top-down’ view of FIG. 13 shows a view of components which are not contained within a single plane. For example, the SWDs 1320, sense amplifiers 1330, and multiplexer drivers 1340 may generally be located in a plane which is above the plane of the word lines, global digit lines, and multiplexers 1310 in the z direction. Certain components in the plane of the array, such as certain multiplexers 1310, and portions of the global digit lines and word lines may be occluded by the sense amplifiers 1330 and multiplexer drivers 1340 which are above the memory patch.

The quilt 1300 shows four memory patches 1312-1318 (e.g., 252g-257g of FIG. 2G and/or 1202-1216 of FIG. 12), SWD/staircase regions 1322 and 1324 (e.g., 172/182 of FIG. 1, 272g/282g and 276g/286g of FIG. 2G and/or 1204 and 1214 of FIG. 12), sense amplifier regions 1332-1338 (e.g., 174 of FIG. 1, 222g-228g of FIG. 2G, and/or 1223-1228 of FIG. 12), multiplexer driver regions 1342-1348 (e.g., 166/176 of FIG. 1, 242g-248g of FIG. 2G, and/or 1252 of FIG. 12), and interpatch regions 1352-1354 (e.g., 1210 of FIG. 12). The array patches 1312-1318 each include a plurality of memory cells not shown in the view of FIG. 5 at the intersection of word lines and local digit lines. The ‘top’ of the local digit lines are selectively coupled through multiplexers 1310 to the associated global digit line. The SWD regions 1322-1324 include SWD circuits 1320. The sense amplifier regions 1332-1338 include sense amplifier circuits 1330. The multiplexer driver regions 1342-1348 include multiplexer driver circuits 1340. The interpatch regions 1352 and 1354 show the top of vertical conductive elements 1350 which couple the global digit lines in the z direction from the xy plane in which they run through the memory patches 1312-1318 up to the xy plane of the sense amplifiers.

The sense amplifier circuits 1330 in the sense amplifier regions 1332 and 1336 are coupled to global digit lines in the patches 1312 and 1316. The sense amplifier circuits 1330 in the sense amplifier regions 1334 and 1338 are coupled to global digit lines in the patches 1314 and 1318. In the sense amplifier regions 1334-1338, the sense amplifiers are arranged in a row extending in the x direction. A first half of the GDLs (both even and odd) in the patches 1312 and 1316 are coupled to the sense amplifiers 1330 in the portions 1336. A second half of the GDLs (both even and odd) in the patches 1312 and 1316 are coupled to the sense amplifiers 1330 in the portion 1332. A first half of the GDLs (both even and odd) in the patches 1314 and 1318 are coupled to the sense amplifiers 1330 in the portions 1338. A second half of the GDLs (both even and odd) in the patches 1314 and 1318 are coupled to the sense amplifiers 1330 in the portion 1334.

In the example of FIG. 13, the halves are organized numerically from a first GDL to the middle GDL, and from the middle GDL to the last GDL when counting across the patches from left to right (or right to left) along the x axis. For example, in the example implementation where there are 1024 GDLs, a first 512 of them from GDL0 to GDL511 are couple to sense amplifiers in the regions 1336 and 1338, while a second 512 of them from GDL512 to GDL51023 are coupled to sense amplifiers in the regions 1332 and 1334. Adjacent sense amplifier circuits 1330 within a portion 1332-1338 may generally be coupled to adjacent GDLs. In the example of FIG. 13, each SA region 1332-1338 includes 512 sense amplifiers, each coupled to two GDLs, one in each of the two associated patches.

The SA portion 1332 is located above the patch 1312 in the z direction, the SA portion 1334 is located above the patch 1334 in the z direction, the SA portion 1336 is located above the patch 1316 in the z direction, and the SA portion 1338 is located above the patch 1318 in the z direction. The SA portions 1332-1338 all border an interpatch region 1352 or 1354. The interpatch regions 1352 is between the patches 1312 and 1316 along the y axis and the interpatch region 1354 is between the patches 1314 and 1318 along the y axis. The SA region 1336 is ‘below’ the interpatch region 1352 in the y direction, while the SA region 1332 is ‘above’ the interpatch region 1352 in the y direction. The SA region 1338 is ‘below’ the interpatch region 1354 in the y direction, while the SA region 1334 is ‘above’ the interpatch region 1354 in the y direction.

The cross section 1300 includes word lines WL0 to WL7999. The word lines are arranged in 80 stacks of 100 word lines each. So the top row of word lines includes the visible word line (WL0) as well as 99 more word lines extending in the z direction into the plane from the point of view of the drawing. Multiplexer circuits 1310 couple a ‘top’ of one or more LDLs to associated GDLs. The LDLs are arranged in a grid layout in an xy plane, with a row of the grid including 1024 LDLs along the x direction and a column of the grid including of 80 LDLs along the y direction. The word lines WL extend from the first patch 1310 to the second patch 1330 under the SWD and staircase region 1320.

The SWD and staircase region 1322-1324 includes a number of SWDs 1320. There is a SWD for each word line. In this example there are 8000 SWDs 1320, arranged in a grid of 80x100 SWDs in the yz plane. The SWDs 1304 are arranged in 80 rows, with each row over a stack of WLs. Since the stacks of WLs are arranged with 100 WLs in each stack, each row of SWDs has 100 SWDs. Each SWD along a row has a conductive element running in the z direction down to the WL it is coupled to. The SWDs along a ‘row’ of the grid of SWDs are coupled to conductive elements of different lengths, since they are coupled to different depths (in the z direction) of word line. When activated by a row activation command, the SWD associated with the row address activates the associated word line.

The LDLs are coupled to the respective GDL by a multiplexer circuit 1310. The multiplexer circuit is generally positioned between a ‘top’ of the LDL in the z direction and the GDL. In the embodiment of FIG. 13, there is a multiplexer circuit 1310 for each LDL. Other example embodiments may group multiple LDLs together through each multiplexer circuit 1310. A multiplexer driver circuit 1340 is coupled by one or more signal lines extending in the x direction to a row of multiplexer driver circuits. The signal lines provide multiplexer enable signals, bleed enable signals or both. A row of multiplexers 1310 may be coupled to a multiplexer driver in common.

In the embodiment of FIG. 13, the multiplexers 1306 are arranged in an xy grid of 80 rows and 1024 columns. Each row of 1024 multiplexers is coupled in common to a multiplexer driver 1340 in an associated one of the multiplexer driver regions 1342-1348. Accordingly, there are 80 multiplexer drivers 1340 in each region 1342-1348. Each region 1342-1348 is associated with one of the patches 1312-1318 and is positioned above (in the z direction) the patch it is associated with. So for example the region 1342 is above the patch 1312, the region 1344 is above the patch 1314, the region 1346 is above the patch 1316 and the region 1348 is above the patch 1318.

The multiplexer regions 1342-1348 each include multiplexer drivers 1340 arranged in a column extending in y direction. Each multiplexer driver 1340 may be associated with a row of multiplexers 1310 extending in the x direction in the associated patch 1312-1318. In other words, each multiplexer driver 1340 may be associated with a ‘stack’ of word lines and specifically the portion of that word line on one side of the SWD region 1322/1324. So, for example a first multiplexer driver MUXD0L may be associated with the portions of WL0-WL99 to the left of SWD region 1322, a second multiplexer driver MUXD1L may be associated with the portions of WL100-WL199 to the left of SWD region 1322 and so forth.

The multiplexer driver regions 1342-1348 are positioned along an edge of the patch opposite the corner that the sense amplifier region 1332-1338 is positioned along. For example, making reference to the layout of the FIG. 13, in the patches 1312 and 1314, the sense amplifier region 1332 and 1334 are positioned above a lower right corner of the respective patches 1312 and 1315, and the multiplexer driver regions 1342 and 1344 are positioned along a left edge of the patches 1312 and 1314. The layout of the patches 1316 and 1318 reverse this, with the sense amplifier regions 1336 and 1338 positioned above an upper left corner of the patches 1336 and 1338 and the multiplexer driver regions 1346 and 1348 positioned above a right edge of the patches 1316 and 1318. Another way of considering the placement of the multiplexer driver regions 1342-1348 is that the regions 1346 and 1344 are positioned along edges of the patch proximal to the associated SWD regions 1322/1324, while the regions 1342 and 1348 are positioned along edges of the patch distal from the associated SWD regions 1322/1324.

In an example operation, when a row activation command is received along with a row address, the SWD 1320 associated with that address activates the associated word line. The associated multiplexer driver 1362 activates the multiplexer circuit 1306 so that the LDLs which intersect the active word line are coupled to the respective GDLs. When the word line is activated, the memory cells along that word line are coupled to the intersecting LDL 1306, and change a voltage of the LDL and the GDL that LDL is coupled to based on the stored charge. The SA coupled to that GDL senses this change and amplifies it during a read operation, or drives a new value onto the GDL in a write operation.

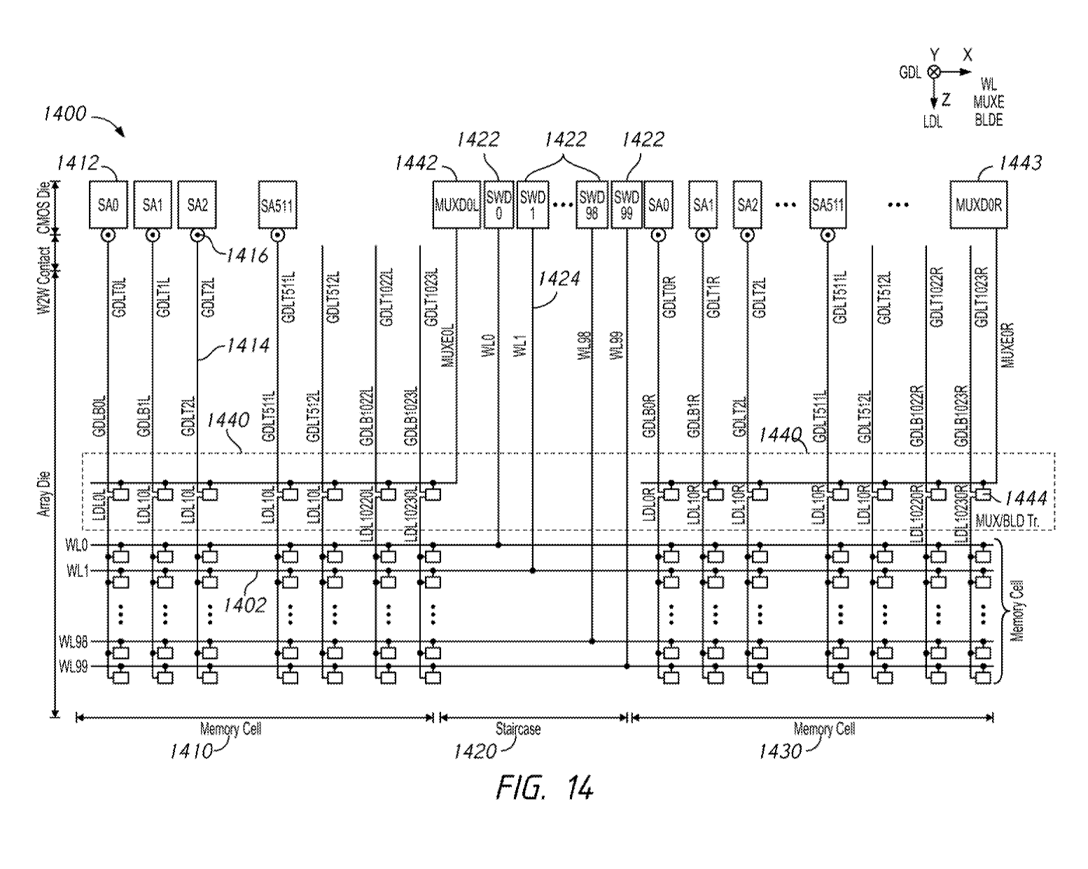

FIG. 14 is a cross sectional view of a memory device according to some embodiments of the present disclosure. The cross section 1400 shows the plane 1400 of FIG. 12. The cross section 1400 represents a view along an example xz plane, showing word lines running horizontally along the x direction, local digit lines running vertically along a z direction, and global digit lines running into and out of the plane of the page along a y direction. A vertical conductive element 1414 is shown in an interpatch region (e.g., 410 of FIG. 4) which runs ‘behind’ the plane of the cross section 1400. The vertical conductive element 1414 is coupled to a horizontal element 1416 which couples to the sense amplifiers 1412, which are positioned over the memory cells. The sense amplifiers 1412 shown are in the sense amplifier portions which are positioned over a left half of the GDLs (e.g., portions 222g and 226g of FIG. 2G, 1226 or 1228 of FIG. 12, and/or 1336 or 1338 of FIG. 13).

The cross section 1400 shows a first memory patch 1410 (e.g., 256g of FIG. 2G, of FIG. 12, and/or 1316 of FIG. 13), a staircase region 1420 (e.g., 286g of FIG. 2G, 1214 of FIG. 12, and/or 1324 of FIG. 13), and a second memory patch 1430 (e.g., 257g of FIG. 2G, 1216 of FIG. 12, and/or 1318 of FIG. 13). The cross section 1400 also shows a row a SWDs 1422 associated with the WLs running through the patches 1410 and 1430.

The view of FIG. 14 shows respective sense amplifiers 1412 positioned above a first half of the memory patches 1410 and 1430. For example, the sense amplifiers SA0 to SA511 are shown coupled to GDLB0L to GDLB511L and GDLT0L to GDLT511L respectively. The other GDLs in the patches 1410 to 1430 from GDLB512L to GDLB1023L and GDLT512L to GDLT1023L are also coupled to sense amplifiers, but those sense amplifiers are in a different cross section not captured by the view of FIG. 6. The view of FIG. 6 shows a row of sense amplifiers, where adjacent sense amplifiers are coupled to adjacent GDLs. In other words, both even and odd GDLs couple to sense amplifiers which are in a same region.

Each word line is coupled to a respective SWD 1422. For example the cross section 1400 shows SWD0 to SWD99, which are associated with WL0 to WL99 respectively. The SWDs 1422 are located above in the z direction a staircase region 1420 which is between the two patches 1410 and 1430 in the x direction. Each SWD 1422 is coupled to a vertical conductive element 1424 which extends in the z direction to the associated word line. Along a row of SWDs 1422 like the one shown in FIG. 6, each of these vertical conductive elements 1424 may be a different length, since the WLs are at different depths in the z direction. In the example layout of FIG. 6, the shortest vertical conductive element, coupled to the ‘top’ word line WL0, is on the far left, while the longest vertical conductive element, coupled to the ‘bottom’ word line WL99, is on the far right. Other arrangements may be used in other example embodiments.

The cross section 1400 shows a ‘stack’ of word lines. In this case the word lines WL0 to WL99. A number of LDLs extend vertically in the z direction and memory cells 1402 are coupled at the intersection of the LDLs and the WLs. In the example implementation, there are 81,920 LDLs per patch from LDL0 to LDL81919. Each LDL seen in the cross section 1400 represents a ‘top’ of a stack of 80 LDLs which extend in the y direction. Thus the leftmost LDL in the patch 1410 is LDL0L, the next LDL is LDL80L, the next is LDL160L and so forth up to LDL81840L. Similarly, the LDLs in the patch 1420 are LDL0R up to LDL81919R.

The view of FIG. 6 includes digit line multiplexers 1444 (e.g., 184 of FIG. 1) in a digit line multiplexer region 1440, and example multiplexer drivers 1442. The multiplexers 1444 selectively couple a set of LDLs to the respective GDLs. In the xz slice shown in FIG. 6, the multiplexers 1444 of the left patch 1410 are coupled to a multiplexer driver 1442 and the multiplexers 1444 of the right patch 1430 are coupled to a multiplexer driver 1443. The multiplexer drivers 1442/1443 are coupled along signal lines which extend in the x direction the multiplexer circuits 1444. For example a first signal line couples the multiplexers 1444 in the patch 1410 to the driver 1442 and a second signal line couples the multiplexers 1444 in the patch 1430 to the driver 1443. The multiplexer drivers 1442/1443 and signal line represent a single row. Additional rows are stacked in the y direction in and out of the plane of the page.

Responsive to a row activation command and a row address, the SWD 1422 specified by the row address activates the coupled word line along the respective vertical conductive element 1424. This couples the memory cells 1402 in the two patches 1410 and 1430 to be coupled to the LDLs which intersect that word line. Responsive to a signal from a global row decoder (e.g., 166 of FIG. 1) based on the row address, the multiplexer drivers 1442 activate the multiplexers 1444 to couple the LDLs along the cross section 1400 to their respective GDLs. The memory cells along the activated WL drive a voltage onto the intersecting LDLs, which then drive a voltage to the coupled GDL through the activated multiplexers 1444. The other LDLs, which are stacked in the Y direction and not visible in FIG. 14, are coupled to multiplexers which are coupled to different multiplexer drivers, and thus are not coupled to the GDLs. This may help reduce the capacitance of the GDL since each GDL is only coupled to one LDL at a time.

In some embodiments, the SWDs 1422 and multiplexer drivers 1442 may be located in a CMOS die (e.g., 212 of FIG. 2B) while the memory cells, LDLs, WLs, and, if used multiplexer region 1440 are located in an array die (e.g., 216 of FIG. 2B). The vertical elements 1424, as well as the vertical portions of the GDLs may extend from the array die to the CMOS die and may include contacts between the two dice not shown in FIG. 6.

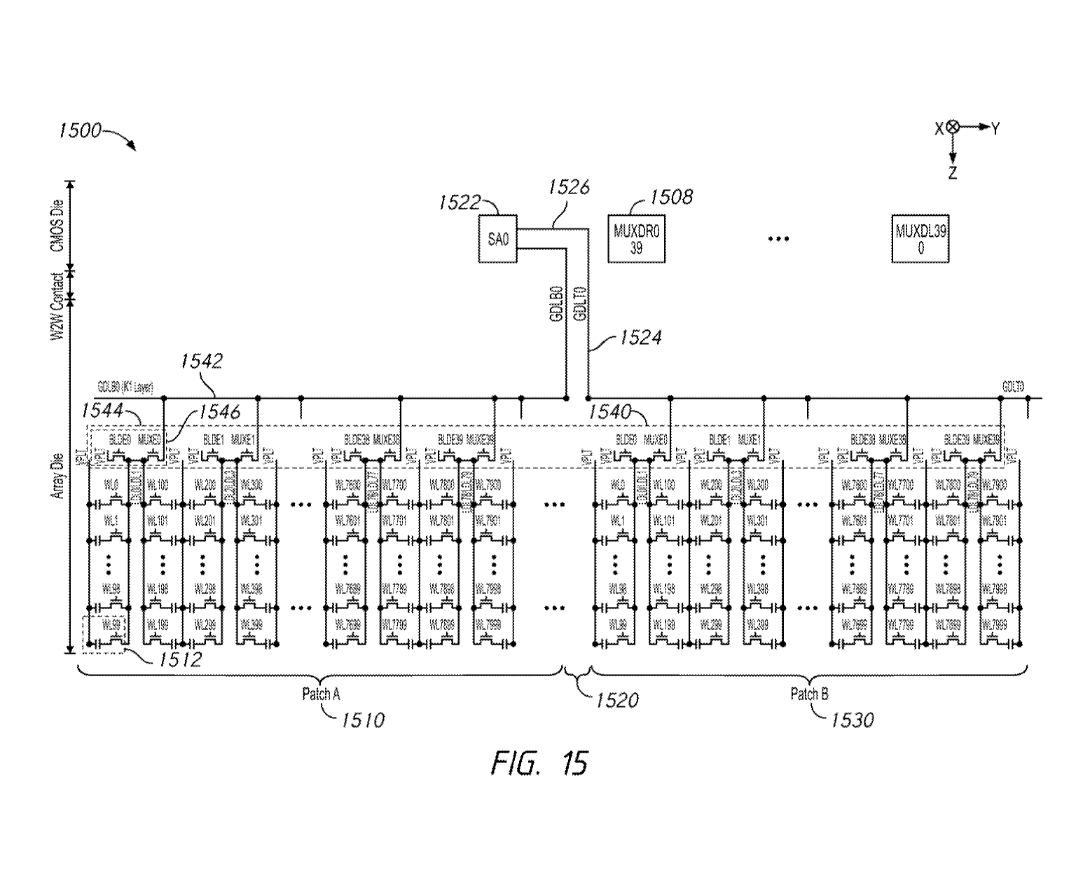

FIG. 15 is a cross sectional view of a memory device according to some embodiments of the present disclosure. The cross section 1500 shows the plane 1500 of FIG. 12. The cross section 1500 represents a view along an example yz plane, showing two global digit lines running horizontally in the y direction, local digit lines running vertically in the z direction, and word lines running through the plane of the page in the x direction. The cross section 1500 shows a first memory patch 1510 (e.g., 256g of FIG. 2G, 1212 of FIG. 12, 1316 of FIG. 13, and/or 1410 of FIG. 14), an interpatch region 1520 (e.g., 1210 of FIG. 12 and/or 1352 of FIG. 13), and a second memory patch 1530 (e.g., 252g of FIG. 2G , 1202 of FIG. 12, and/or 1312 of FIG. 13).

The cross section 1500 intersects 8000 word lines in each memory patch 1510 and 1530. The word lines are organized in a grid in the yz plane with eighty columns in the z direction that have 100 word lines each. Accordingly, the cross section shows 16,000 memory cells 1512, 8000 in each patch 1510 and 1530, with each patch organized in a grid of 80 memory cells in the x direction and 100 memory cells in the z direction. Each column of memory cells 1512 is coupled to a local digit line, here labelled LDL0 to LDL79. In the embodiment of FIG. 15, the LDLs are paired and each pair LDLs is coupled in common to a multiplexer circuit 1546. In some embodiments, each LDL may couple separately through a multiplexer to the GDL, rather than being paired.

Each memory cell 1512 includes a capacitive element and a transistor. The gate of the transistor is coupled to the word line. One side of the capacitive element is coupled to a plate voltage VPLT, and the other side is coupled through the transistor to the LDL. When the word line is activated, the SWD drives a voltage along the word line which activates the transistor, coupling the capacitive element to the LDL.

The cross section 1500 shows a sense amplifier 1522. The sense amplifier 1522 is labelled as SA0 because it is coupled to GDL0 in two adjacent patches 1510 and 1530. To distinguish them, those GDLs are labelled as GDLB0 in the first patch 1510 and GDLT0 in the second patch 1530. During an example operation, one of the two GDLs is used to carry information, and the other is used as a reference. For example, if a word line is activated in the first patch 1510, then information is carried along GDLB0 and GDLT0 is used as a reference. During a read operation, the sense amplifier senses a difference between the GDL with information and the reference and then amplifies that difference. During a write operation, the sense amplifier drives one GDL to a voltage representing the write value and the other to the compliment of the write value.

The sense amplifier 1522 is shown positioned above the memory patch 1510. Vertical conductive elements 1524 in the interpatch region 1520 couple the GLDs GDLB0 and GDLT0 up from the array die to the CMOS die. Horizontal conductive elements 1526 couple the vertical conductive elements from the interpatch region 1520 to the location of the sense amplifier 1522 over the array patch 1510.

FIG. 15 shows multiplexer region 1540 along with the multiplexer drivers 1508 which operates the multiplexer region. The multiplexer circuits 1546 are implemented by a multiplexer transistor 1542 and an optional bleed transistor 1544. Each pair of LDLs is coupled to the GDL through a multiplexer transistor 1542. The gate of the multiplexer transistor 1542 is coupled to a multiplexer control signal MUXE which is coupled to respective multiplexer drivers 1508 along a signal line which runs in the x direction. The bleed transistor 1544 selectively couples each pair of LDL to the plate voltage VPLT. The bleed transistors 1544 have gates coupled to bleed control signals BLDE which are coupled to respective multiplexer drivers 1508 along signal lines which run in the x direction. The multiplexer drivers 1508 are controlled by global row decoders (e.g., 166 of FIG. 1).

During an example operation, if a row address is received associated with the first patch 1510, then the multiplexer driver 1508 which controls the LDL which intersects the WL activates the multiplexer transistor 1542 by providing MUXE, and each of the other multiplexer drivers 1508 provide BLDE to their respective bleed transistor 1544 to couple those LDLs to VPLT and prevent them from floating. For example, if the row address indicates WL 101, which intersects LDL1, then MUXD0 provides MUXE0, which couples both LDL0 and LDL1 to GDLB0. The multiplexer drivers MUX1 to MUX4 provide BLDE1 to BLDE4 respectively, which couple the other LDLs LDL2 to LDL9 to VPLT through the respective bleed transistors 1544.

In some embodiments, multiplexers may be activated on both sides of the sense amplifier in order to match capacitance between the signal and reference GDLs. Using the example above, a row address is received which indicates WL101 in patch 1510. Accordingly, the signal will be along GDLB0 and the reference will be GDLT0. The multiplexer drivers MUXDL0 and MUXDR0 provide the signals MUXE0 to both patches 1510 and 1530. This causes the LDLs LDL0 and LDL1 in both patches 1510 and 1530 to be coupled to GDLB0 and GDLT0 respectively. The multiplexer driver MUXDL0 and MUXDR0 provide BLDE0 at an inactive level. The other multiplexer drivers MUXDL1 to MUXDL39 and MUXDR1 to MUXDR39 provide MUXE1 to MUXE39 at an inactive level and BLDE1 to BLDE39 at an active level.

FIG. 16 is a perspective view of a memory quilt of a memory device according to some embodiments of the present disclosure. The memory quilt 1600 may represent a portion of a memory device such as 100 of FIG. 1, for example the quilt layout 250b of FIG. 2B. The memory quilt 1600 may include one or more memory arrays which have layouts similar to the memory array 300 of FIG. 3 in some embodiments. The memory quilt 1600 may be generally similar to the quilt 250b of FIG. 2B. However, FIG. 16 shows a perspective view rather than a ‘top down’ view of the xy plane.

The memory device which includes the quilt 1600 includes a first die 1640 (e.g., 212b of FIG. 2B) and a second die 1650 (e.g., 216b of FIG. 2B). The first die 1640 may includes the sense amplifiers, SWDs, and multiplexer drivers. For example the first die 1640 may be a CMOS die. The second die 1650 includes the memory array patches and staircase regions.

The memory quilt 1600 includes a first memory patch 1602 (e.g., 252b of FIG. 2B), a second memory patch 1606 (e.g., 254b of FIG. 2B) and a SWD region 1604 (e.g., 172 of FIG. 1, 272b of FIG. 2B, and/or 306 of FIG. 3) in between. Each memory patch has an associated sense amplifier and column decoder region and an associated multiplexer driver and row decoder region. The patch 1602 is associated with sense amplifier and column decoder region 1622 (e.g., 174 and 164 of FIG. 1, 222b and 232b of FIG. 2B, and/or 310 of FIG. 3) and multiplexer driver and row decoder region 1652 (e.g., 176 and 162 of FIG. 1, 242b and 262b of FIG. 2B, and/or 314 of FIG. 3). The patch 1604 is associated with sense amplifier and column decoder region 1624 (e.g., 174 and 164 of FIG. 1, 224b and 234b of FIG. 2B, and/or 311 of FIG. 3) and multiplexer driver and row decoder region 1654 (e.g., 176 and 162 of FIG. 1, 244b and 264b of FIG. 2B, and/or 316 of FIG. 3).

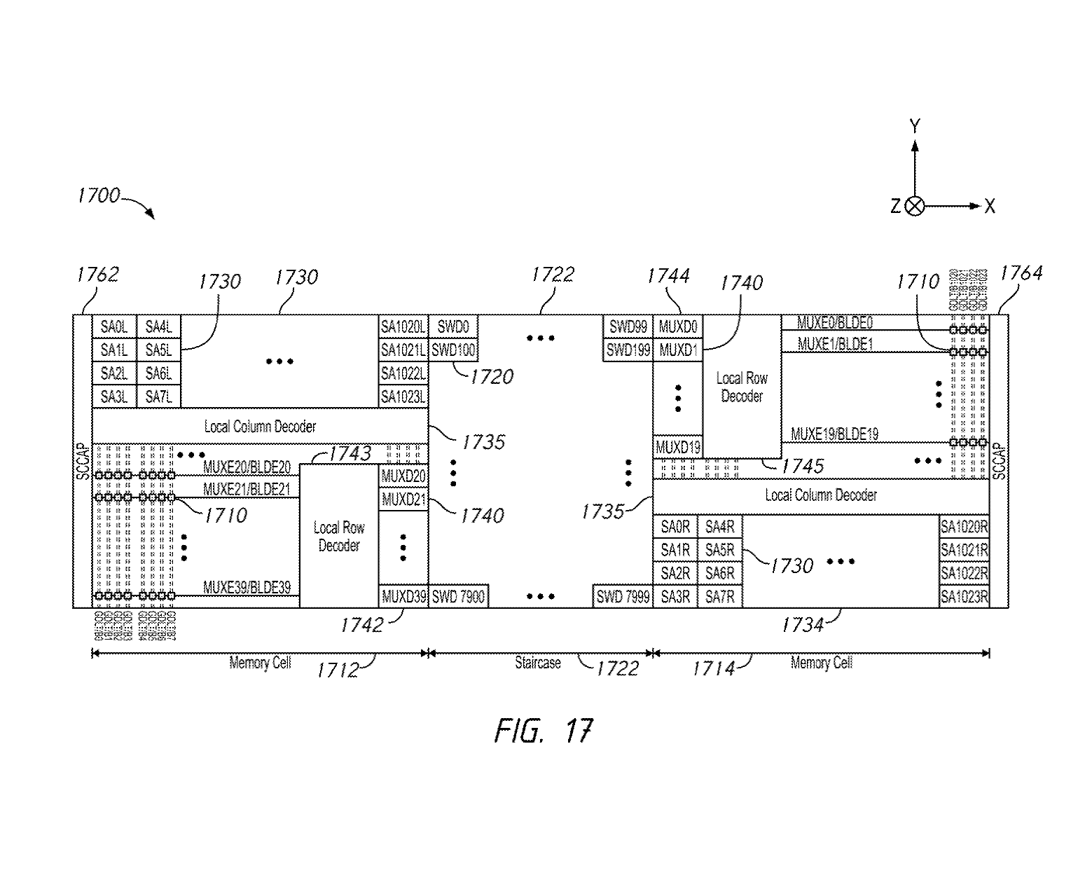

FIG. 16 shows planes 1700, 1800, 1900, and 2000 which represent the cross-sectional views of FIGS. 17, 18, 19, and 20 respectively. The plane 1700 is a slice along an xy plane of the memory 1600 shows the memory patches 1602 and 1604 as well as the sense amplifier/column decoder regions 1622 and 1624, the multiplexer driver/row decoder regions 1652 and 1654 and the SWD and staircase region 1610.

The plane 1800 is a slice along an xz plane of the memory which intersects a second memory patch 1602, the SWD and staircase region 1610 and a second memory patch 1604. The plane 1800 also intersects sense amplifier region 1622. The plane 1900 is a slice along a yz plane which intersects the first memory patch 1602 and the sense amplifier region 1622. The plane 2000 is a similar yz slice to the plane 1900 except that the plane 2000 is slightly offset from the plane 1900 in the x direction.

FIGS. 17-20 show different cross-sectional views of the memory device 1600 of FIG. 16. Each of FIGS. 17-20 is illustrated with respect to an example embodiment where each memory patch such as 1602 and 1606 includes 8 Mbit of memory cells. Specifically, they are shown to include a grid of 80 word lines in the y direction and 100 word lines in the z direction, a grid of 80 LDLs in the y direction and 1024 LDLs in the x direction, and 1024 pairs of global digit lines side-by-side. This arrangement is shown as an illustrative example only. Other numbers and/or arrangements of word lines, global digit lines, local digit lines, memory cells, sense amplifiers, and so forth may be used in other example embodiments.

FIG. 17 is a top-down view of a memory quilt according to some embodiments of the present disclosure. The quilt 1700 shows a view of an example quilt such as 202a of FIG. 2A and/or 1600 of FIG. 16. The cross section 1700 shows the plane 1700 of FIG. 16. The quilt 1700 represents a view along an example xy plane, showing a top layer of word lines extending horizontally in an x direction, global digit lines extending in a y direction, and local digit lines extending into the plane of the page in the z direction from the multiplexer circuits 1710 where they intersect the GDL. The ‘top-down’ view of FIG. 17 shows a view of components which are not contained within a single plane. For example, the SWDs 1720, sense amplifiers 1730, multiplexer drivers 1740, and the row decoders 1743/1745 and column decoders 1733/1735 may generally be located in a plane which is above the plane of the word lines, global digit lines, and multiplexers 1710 in the z direction.

Certain components in the plane of the array, such as certain multiplexers 1710, and portions of the global digit lines and word lines may be occluded by the sense amplifiers 1730 and multiplexer drivers 1740 which are above the memory patch. For example, the view of FIG. 5 shows global digit line pairs and multiplexer enable signal lines viewed through the spacer regions (e.g., 258 of FIG. 2) into the array patches (e.g., 252/254 of FIG. 2) below. The view of the components of the array patch visible in the view of FIG. 5 shows the multiplexer enable and bleed enable signal lines MUXE/BLDE and multiplexers 1710 which run along a top of the array (in the z direction). The word lines may generally be stacked vertically below the MUXE/BLDE lines in the z direction, and the local digit lines and memory cells run below the multiplexers 1710.

The quilt 1700 shows two memory patches 1712 and 1714 (e.g., 252b/254b of FIG. 2B and/or 1604/1604 of FIG. 16), a SWD/staircase region 1722 (e.g., 172/182 of FIG. 1, 272b/282b of FIG. 2B, and/or 1604 and 1614 of FIG. 16), sense amplifier regions 1732 and 1734 (e.g., 174 of FIG. 1, 222b/224b of FIG. 2B, 310/311 of FIG. 3, and/or 1622-1624 of FIG. 16), multiplexer driver regions 1742 and 1744 (e.g., 176 of FIG. 1, 242b and 244b of FIG. 2B, 314/316 of FIG. 3, and/or 1652/1654 of FIG. 16). Also shown in FIG. 17 are the local row decoders 1743 and 1745 (e.g., 162 of FIG. 1, and/or 262b/264b of FIG. 2B) and local column decoders 1735 (e.g., 164 of FIG. 1 and/or 232b/234b of FIG. 2B).

The SWD regions 1722 includes SWD circuits 1720. The sense amplifier regions 1732 and 1734 include sense amplifier circuits 1730. The multiplexer driver regions 1742 and 1744 include multiplexer driver circuits 1740. The array patches 1712 and 1714 each include a plurality of memory cells not shown in the view of FIG. 17 at the intersection of word lines and local digit lines. The ‘top’ of the local digit lines are selectively coupled through multiplexers 1710 to one of the global digit lines of the associated global digit line pair. In the view of FIG. 5, the global digit line pairs are represented as a double line, one solid and one dotted, which are side-by-side in the xy plane. However, this is to help represent the paired nature of the GDLs. It does not necessarily represent the spatial arrangement of the GDL pair. For example, in some embodiments the two paired GDLs may be side by side in an xy plane, however in other embodiments the two paired GDLs may be above each other in a yz plane. Both GDLs of the pair are coupled to a same sense amplifier 1730.

The sense amplifier circuits 1730 in the sense amplifier regions 1732 and 1734 are coupled to both GDLs of a GDL pair. The GDL pair includes two GDLs labelled GDLT and GDLB. During an operation, the GDL of the GDL pair which is coupled to the active word line is used as a signal line and the other is used as a reference. For example, if the word line is coupled to GDLB, then the sense amplifier uses GDLT as a reference. Each patch includes 1024 GDL pairs, each coupled to one of 1024 sense amplifiers 1730. In the example of FIG. 5, the sense amplifier regions 1732 and 1734 are arranged in grids with 4x256 sense amplifiers in the y and x directions respectively. For example, a first column includes SA0 coupled to GDLT0 and GDLB0, SA1 coupled to GDLT1 and GDLB1, SA2 coupled to GDLT2 and GDLB2, and SA3 coupled to GDLT3 and GDLB3. Vertical conductive elements (not shown in FIG. 17) extending in the z direction couple from the GDL pair up to the coupled sense amplifier.

The memory array includes 8000 word lines (not shown), WL0 to WL7999 . The word lines are arranged in 80 stacks of 100 word lines each. So the top row of word lines includes the visible word line (WL0) as well as 99 more word lines extending in the z direction into the plane from the point of view of the drawing. Multiplexer circuits 1710 couple a ‘top’ of one or more LDLs to associated GDLs. The LDLs are arranged in a grid layout in an xy plane, with a row of the grid including 1024 LDLs along the x direction and a column of the grid including of 80 LDLs along the y direction. Each LDL is coupled through the multiplexer to one or other of the GDL pair. Which GDL of the GDL pair the LDLs are coupled to may change along the length of the GDL pair. The word lines WL extend from the first patch 1712 to the second patch 1714 under the SWD and staircase region 1722.

The SWD and staircase region 1722 includes a number of SWDs 1720. There is a SWD for each word line. In this example there are 8000 SWDs 1720, arranged in a grid of 80x100 SWDs in the yz plane. The SWDs 1720 are arranged in 80 rows, with each row over a stack of WLs. Since the stacks of WLs are arranged with 100 WLs in each stack, each row of SWDs has 100 SWDs. Each SWD along a row has a conductive element running in the z direction down to the WL it is coupled to. The SWDs along a ‘row’ of the grid of SWDs are coupled to conductive elements of different lengths, since they are coupled to different depths (in the z direction) of word line. When activated by a row activation command, the SWD associated with the row address activates the associated word line.

The LDLs are coupled to the respective one of the GDL pair by a multiplexer circuit 1710. The multiplexer circuit is generally positioned between a ‘top’ of the LDL in the z direction and the GDL. In the embodiment of FIG. 17, each multiplexer circuit 1710 is associated with a pair of LDLs which are coupled in common through the multiplexer 1710 to the associated one of the GDL pair. Other arrangements, such as a multiplexer for each LDL, a multiplexer for every 3 LDLs, etc., may be used in other example embodiments. A multiplexer driver circuit 1740 is coupled by one or more signal lines extending in the x direction to a row of multiplexer driver circuits. The signal lines provide multiplexer enable signals MUXE, bleed enable signals BLDE or both. A row of multiplexers 1710 may be coupled to a multiplexer driver in common.

In the embodiment of FIG. 17, the multiplexers 1706 are arranged in an xy grid of 40 rows and 1024 columns. Each row of 1024 multiplexers is coupled in common to a multiplexer driver 1740 in an associated one of the multiplexer driver regions 1742-1748. Accordingly, there are 40 multiplexer drivers 1740 in each region 1742 and 1744. Each region 1742-1748 is associated with both of the regions 1712 and 1714. The region 1742 is above the patch 1712 and the region 1744 is above the patch 1714.

The multiplexer regions 1742-1748 each include multiplexer drivers 1740 arranged in a column extending in y direction. Each multiplexer driver 1740 may be associated with a row of multiplexers 1710 extending in the x direction in the associated patch 1712-1718. In other words, each multiplexer driver 1740 may be associated with one or more ‘stack’ of word. So, for example a first multiplexer driver MUXD0 may be associated with WL0 to WL199 since in the embodiment of FIG. 5 each multiplexer driver is associated with two stacks of word lines.

FIG. 17 also shows a pair of SCCAP regions 1762 and 1764 (e.g., 110 of FIG. 1) which are positioned on the edges of the memory quilt 1700. The two SCCAP regions 1762 and 1764 are positioned on opposite edges of the memory quilt 1700 in the x direction.

In an example operation, when a row activation command is received along with a row address, the SWD 1720 associated with that address activates the associated word line. The associated multiplexer driver 1740 activates the multiplexer circuit 1710 so that the LDLs which intersect the active word line are coupled to the respective one of the pair of associated GDLs. When the word line is activated, the memory cells along that word line are coupled to the intersecting LDL and change a voltage of the LDL and the GDL that LDL is coupled to based on the stored charge. The SA coupled to that GDL senses this change by comparing the voltage on the GDL of the pair which is coupled to the word line to a pre-charge voltage on the other GDL of the pair and amplifies the difference during a read operation, or drives a new value onto the GDL pair in a write operation.

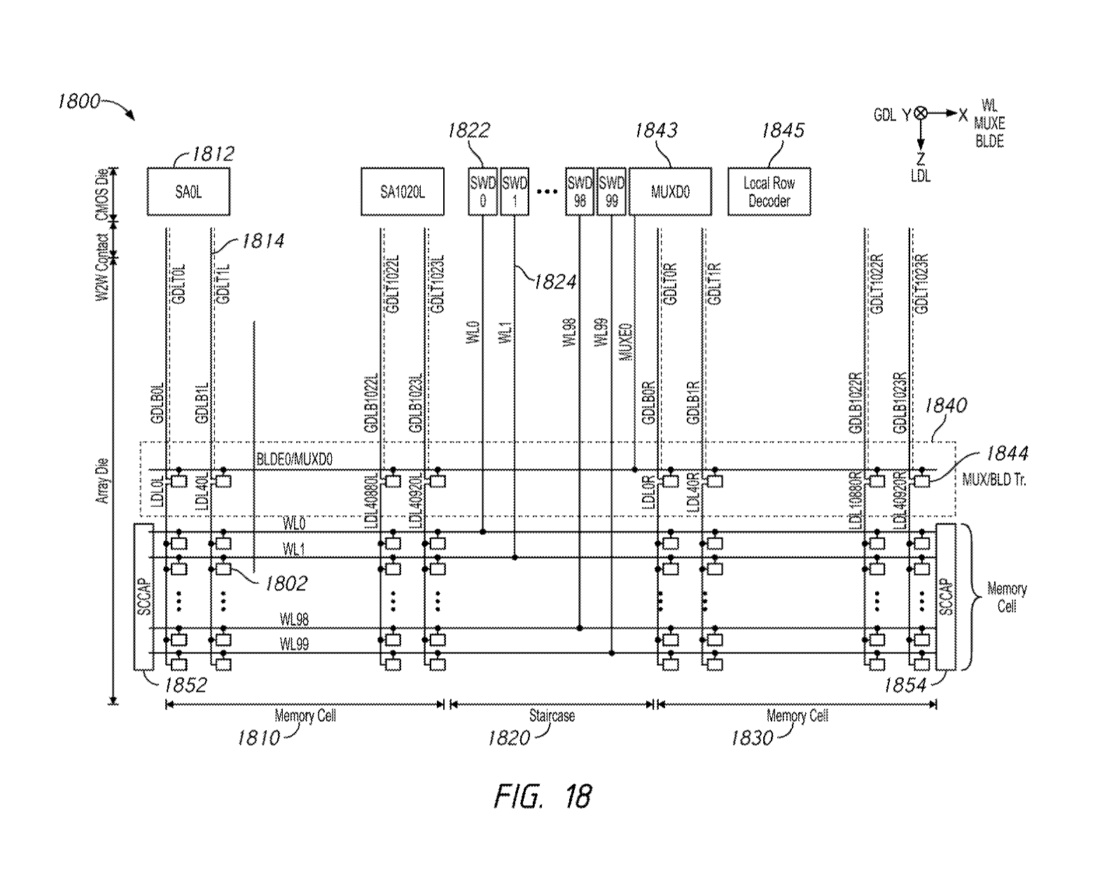

FIG. 18 is a cross sectional view of a memory device according to some embodiments of the present disclosure. The cross section 1800 shows the plane 1800 of FIG. 16. The cross section 1800 represents a view along an example xz plane, showing word lines running horizontally along the x direction, local digit lines running vertically along a z direction, and global digit line pairs running into and out of the plane of the page along a y direction. Pairs of vertical conductive elements 1814 are shown which runs ‘behind’ the plane of the cross section 1800. The vertical conductive elements 1814 are coupled to the sense amplifiers 1812, which are positioned over the memory cells. The sense amplifiers 1812 shown are in the sense amplifier portions which are positioned over the first memory patch 1810 (e.g., sense amplifier regions 222b of FIG. 2B, 310 of FIG. 3, 1622 of FIG. 16, and/or 1732 of FIG. 7). Each sense amplifier 1812 represents a ‘front’ sense amplifier in a column of sense amplifiers that extends out of the plane of the page.

The cross section 1800 shows a first memory patch 1810 (e.g., 252b of FIG. 2B, 1602 of FIG. 16, and/or 1712 of FIG. 17), a staircase region 1820 (e.g., 286b of FIG. 2B, 1610 of FIG. 16, and/or 1722 of FIG. 17), and a second memory patch 1830 (e.g., 254b of FIG. 2B, 1604 of FIG. 16, and/or 1714 of FIG. 17). The cross section 1800 shows a row a SWDs 1822 associated with the WLs running through the patches 1810 and 1830, a multiplexer driver 1843 (e.g., 176 of FIG. 1 , 244b of FIG. 2B, 316 of FIG. 3, 1654 of FIG. 16, and/or 1744 of FIG. 17) and local row decoder 1845 (e.g., 162 of FIG. 1, 264b of FIG. 2B, and/or 1745 of FIG. 17).

Each word line is coupled to a respective SWD 1822. For example, the cross section 1800 shows SWD0 to SWD99, which are associated with WL0 to WL99 respectively. The SWDs 1822 are located above in the z direction a staircase region 1820 which is between the two patches 1810 and 1830 in the x direction. Each SWD 1822 is coupled to a vertical conductive element 1824 which extends in the z direction to the associated word line. Along a row of SWDs 1822 like the one shown in FIG. 6, each of these vertical conductive elements 1824 may be a different length, since the WLs are at different depths in the z direction. In the example layout of FIG. 6, the shortest vertical conductive element, coupled to the ‘top’ word line WL0, is on the far left, while the longest vertical conductive element, coupled to the ‘bottom’ word line WL99, is on the far right. Other arrangements may be used in other example embodiments.

The cross section 1800 shows a ‘stack’ of word lines. In this case the word lines WL0 to WL99. A number of LDLs extend vertically in the z direction and memory cells 1802 are coupled at the intersection of the LDLs and the WLs. In the example implementation, there are 81,920 LDLs per patch from LDL0 to LDL81919. Each LDL seen in the cross section 1800 represents a ‘top’ of a stack of 80 LDLs which extend in the y direction. Thus the leftmost LDL in the patch 1810 is LDL0L, the next LDL is LDL80L, the next is LDL160L and so forth up to LDL81840L. Similarly, the LDLs in the patch 1820 are LDL0R up to LDL81919R.

The view of FIG. 18 includes digit line multiplexers 1844 (e.g., 184 of FIG. 1, 318 of FIG. 3, and/or 1710 of FIG. 17) in a digit line multiplexer region 1840, and example multiplexer drivers 1843. The multiplexers 1844 selectively couple a set of LDLs to one of the GDLs of the respective GDLs. The multiplexer driver 1843 is coupled to one or more signal lines extending the X direction which carry the multiplexer enable signal MUXE and the bleed enable signal BLDE which control the multiplexer region 1840. For example, a first signal line couples the multiplexers 1844 to MUXE and a second signal line couples the multiplexers 1844 to BLDE. The multiplexer driver 1843 represents a single driver in a multiplexer region. In particular, in the view 1800 of FIG. 618, the driver is MUXD0, which provides signals MUXE0 and BLDE0. Additional drivers are stacked through the plane of the page in the y direction.

Responsive to a row activation command and a row address, local row decoder 1845 selects a SWD 1822 and multiplexer driver 1843 based on the row address. For example, if the row address is associated with WL1, then MUXD0 will provide MUXE0, and the SWD1 1822 will provide an enable signal WL1. The selected multiplexer driver activates a set of multiplexers 1840 to couple the LDLs to one of the pair of GDLs. The selected SWD 1822 activates the associated word line. This couples the memory cells 1802 in the two patches 1810 and 1830 to be coupled to the LDLs which intersect that word line. The memory cells along the activated WL drive a voltage onto the intersecting LDLs, which then drive a voltage to the coupled GDL of the pair of GDLs through the activated multiplexers 1844. The other LDLs, which are stacked in the Y direction and not visible in FIG. 18, are coupled to multiplexers which are coupled to different multiplexer drivers, and thus are not coupled to the GDLs. This may help reduce the capacitance of the GDL since each GDL is only coupled to the selected LDLs during a given operation a time.

In some embodiments, the SWDs 1822 and multiplexer drivers 1842 may be located in a CMOS die (e.g., 212a of FIG. 2A) while the memory cells, LDLs, WLs, and, if used multiplexer region 1840 are located in an array die (e.g., 216a of FIG. 2A). The vertical elements 1824, as well as the vertical portions of the GDLs may extend from the array die to the CMOS die and may include contacts between the two dice not shown in FIG. 6.

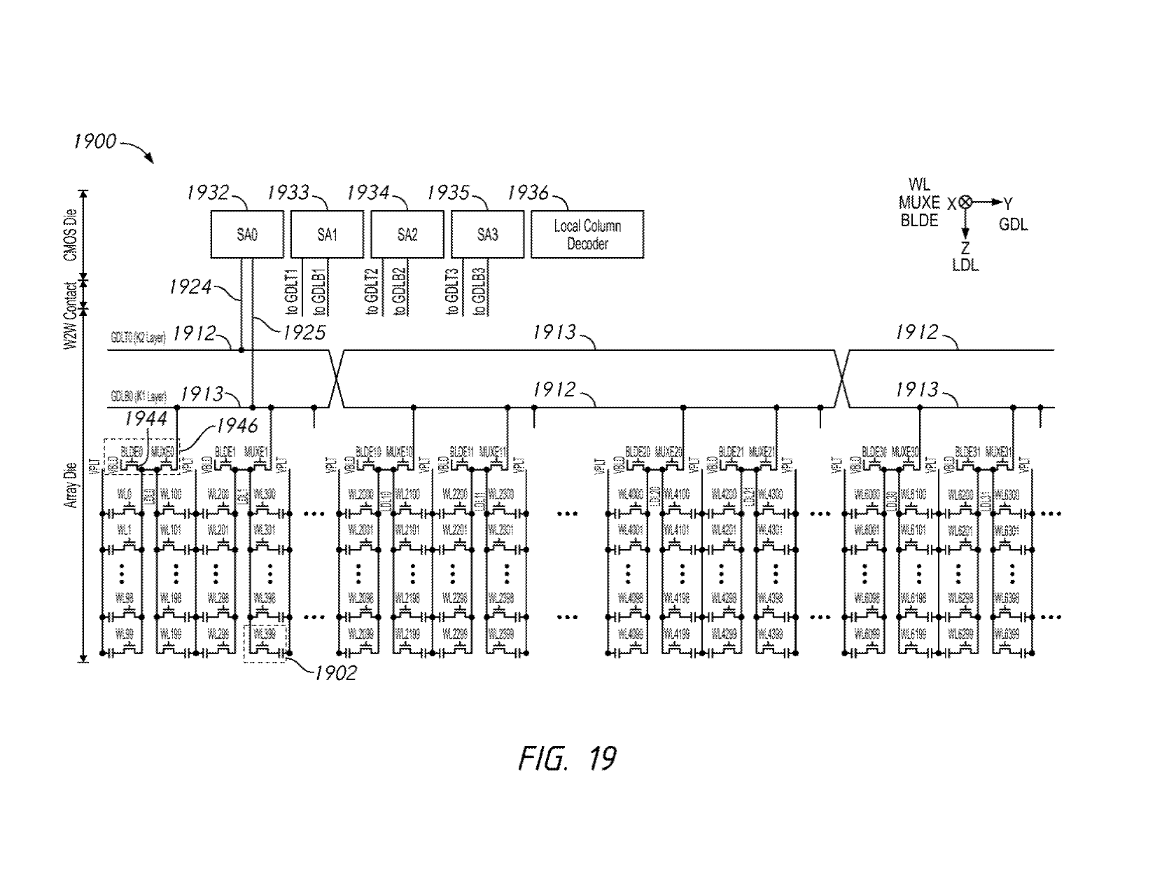

FIG. 19 is a cross sectional view of a memory device according to some embodiments of the present disclosure. The cross section 1900 shows the plane 1900 of FIG. 16. The cross section 1900 represents a view along an example yz plane, showing a pair of global digit lines 1912 and 1913 running horizontally in the y direction, local digit lines running vertically in the z direction, and word lines running through the plane of the page in the x direction. The cross section 1900 shows a portion of a first memory patch 1910 (e.g., 252b of FIG. 2B, 1602 of FIG. 16, 1712 of FIG. 17, and/or 1810 of FIG. 18).

The cross section 1900 intersects 8000 word lines in the memory patch. The word lines are organized in a grid in the yz plane with eighty columns in the z direction that have 100 word lines each. The 8000 word lines in the patch are organized in a grid of 80 memory cells in the x direction and 100 memory cells in the z direction. Each column of memory cells 1902 is coupled to a local digit line, here labelled LDL0 to LDL79. In the embodiment of FIG. 19, the LDLs are paired and each pair LDLs is coupled in common to a multiplexer circuit 1942. In some embodiments, each LDL may couple separately through a multiplexer to the GDL, rather than being paired.

Each memory cell 1902 includes a capacitive element and a transistor. The gate of the transistor is coupled to the word line. One side of the capacitive element is coupled to a plate voltage VPLT, and the other side is coupled through the transistor to the LDL. When the word line is activated, the SWD drives a voltage along the word line which activates the transistor, coupling the capacitive element to the LDL.

The cross section 1900 shows sense amplifiers 1932, 1933, 1934, and 1935. The sense amplifiers are part of a column of sense amplifiers in the y direction in a sense amplifier region. Each sense amplifier 1932-1935 is coupled to a different GDL pair. For example, the sense amplifier SA0 is coupled to GDLT0 1912 and GDLB0 1913, which form a sense amplifier pair. During an example operation, one of the two GDLs 1912/1913 is used to carry information, and the other is used as a reference. Along the length of the two GDLs, different LDLs are coupled to either the first or the second GDL of the pair. For example, as shown in FIG. 7, LDL0 to LDL9 may be coupled to GDLB0 1913, LDL10 to LDL29 may be coupled to GDLT01912, LDL30 to LDL49 may be coupled to GDLT0 1912, and so forth.

In some example embodiments, such as the one shown in FIG. 7, the two GDLs in the pair may be stacked in the z direction, with one GDL over the top of the other. For example one GDL of the pair may be in one metal layer, and the other GDL may be in a second metal layer. In some embodiments, the GDLs of the pair may ‘twist’ or switch which GDL of the pair is in which metal layer. For example, in the embodiment of FIG. 7, GDLT0 is above GDLB0 when the pair runs above LDL0 to LDL9, but then the GDLs twist and GDLB0 is above GDLT0 from LDL10 to LDL19. This may be useful to allow the LDLs to always couple to the lower of the two GDLs (e.g., the GDL closer to the LDLs), but allow for changes in which LDLs are coupled to which of the GDL pair along its length. In this manner, portions of both of the GDLs 1912 and 1913 are in both metal layers.

The sense amplifiers 1932-1935 are shown positioned above at least some of the memory cells 1902. Vertical conductive elements 1924 and 1925 couple the GDLs of the pair GDLB0 1913 and GDLT0 1912 up from the array die to the CMOS die. Since the vertical conductive elements are also over the array and the memory cells 1902, the vertical conductive elements may run directly up from the array die to the sense amplifiers, without the need for additional horizontal (e.g., in the xy plane) routing. The vertical conductive elements 1924 and 1925 may have different lengths (e.g., analogous to the staircase region) since they run to different ‘depths’ along the z direction. FIG. 7 also shows a local column decoder 1936 (e.g., 164 of FIG. 1, 232b of FIG. 2B, and/or 1733 of FIG. 17) which determines which GDL pair is coupled to the global input/output lines during an operation.

Each multiplexer circuit 1942 includes a multiplexer transistor 1946 and a bleed transistor 1944. The bleed transistor 1944 has a gate coupled to a bleed enable signal BLDE provided by a multiplexer driver and terminals coupled between a ground voltage VBLD and the pair of local digit lines. The multiplexer transistor 1946 has a gate coupled to a multiplexer enable signal MUXE and terminals coupled between one of the GDLs and the pair of LDLs.

During an example operation, if a row address is received associated with the first patch 1910, then the multiplexer driver which controls the LDL which intersects the WL activates the multiplexer transistor 1946 by providing MUXE, and each of the other multiplexer drivers provide BLDE to their respective bleed transistor 1944 to couple those LDLs to VBLD and prevent them from floating. For example, if the row address indicates WL 101, which intersects LDL0, then MUXD0 provides MUXE0, which couples LDL0 to GDLB0 1913. The other LDLs receive BLDE at an active level, (e.g., BLDE1 to BLDE39) which couples those LDLs to VBLD instead of to the respective GDL.

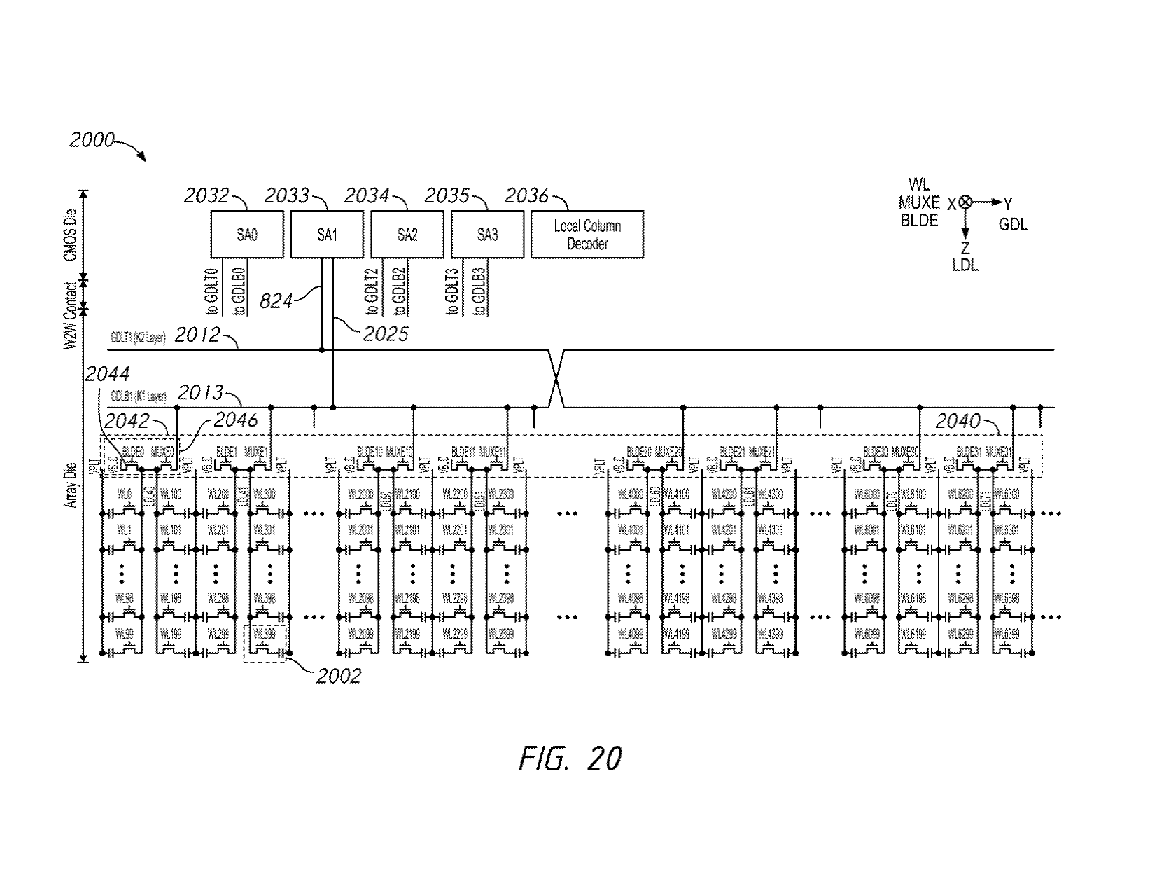

FIG. 20 is a cross sectional view of a memory device according to some embodiments of the present disclosure. The cross section 2000 is generally similar to the cross section 1900 of FIG. 19, except that the cross section 2000 is offset from the cross section 700 in the +x direction and includes the adjacent pair of GDLs GDLT1 2012 and GDLB1 2013. For the sake of brevity certain details which were already previously described with respect to FIG. 19 are not repeated again with respect to FIG. 20. FIG. 20 uses similar reference numbers for similar components, and reuses the same reference numbers as FIG. 19 for components which extend through the x direction to be visible in both cross sections 1900 and 2000.

In the cross-section 2000 of FIG. 20, a different GDL pair is shown. The GDL pair GDLT12012 and GDLB12013 are coupled along vertical elements 2024 and 2025 (respectively) to SA1 1933. Also, the GDL pair of GDLT/B1 twists in different places than the GDL pair of GDLT/B0 of FIG. 19. For example, the GDL pair GDLT12012 and GDLB12013 twist between LDL59 and LDL60 instead of between LDL9 and LDL10.

The present disclosure is drawn to apparatuses, systems, and methods for a refresh scheme that refreshes word lines in a same tier in 3D-DRAM. A 3D memory device may include a 3D memory array in an array patch portion of a memory quilt. The 3D memory array may be refreshed on a word line-by-word line basis, where the memory cells along each word line are refreshed periodically. For example, during a refresh operation, the memory cells along one or more word lines are refreshed. The frequency at which the word lines are refreshed, or the maximum time any given word line will go between refreshes, may be determined based on a refresh specification.

Responsive to a refresh signal, the 3D memory device may perform a refresh operation and one or more word lines in the 3D memory array may be refreshed. For example, during a refresh operation, a word line in one or more memory quilts of a 3D memory device may be refreshed at a time. The word lines being refreshed may all be at a same tier of the 3D memory device. The tier may represent the depth at which the refreshed word line exists within a stack of word lines in the 3D memory array. Refreshing word lines at the same tier across the 3D memory bank may be achieved by the 3D memory device activating the sub-word line drivers one-by-one. The 3D memory device may refresh all of the word lines by iterating through the sub-word line drivers.

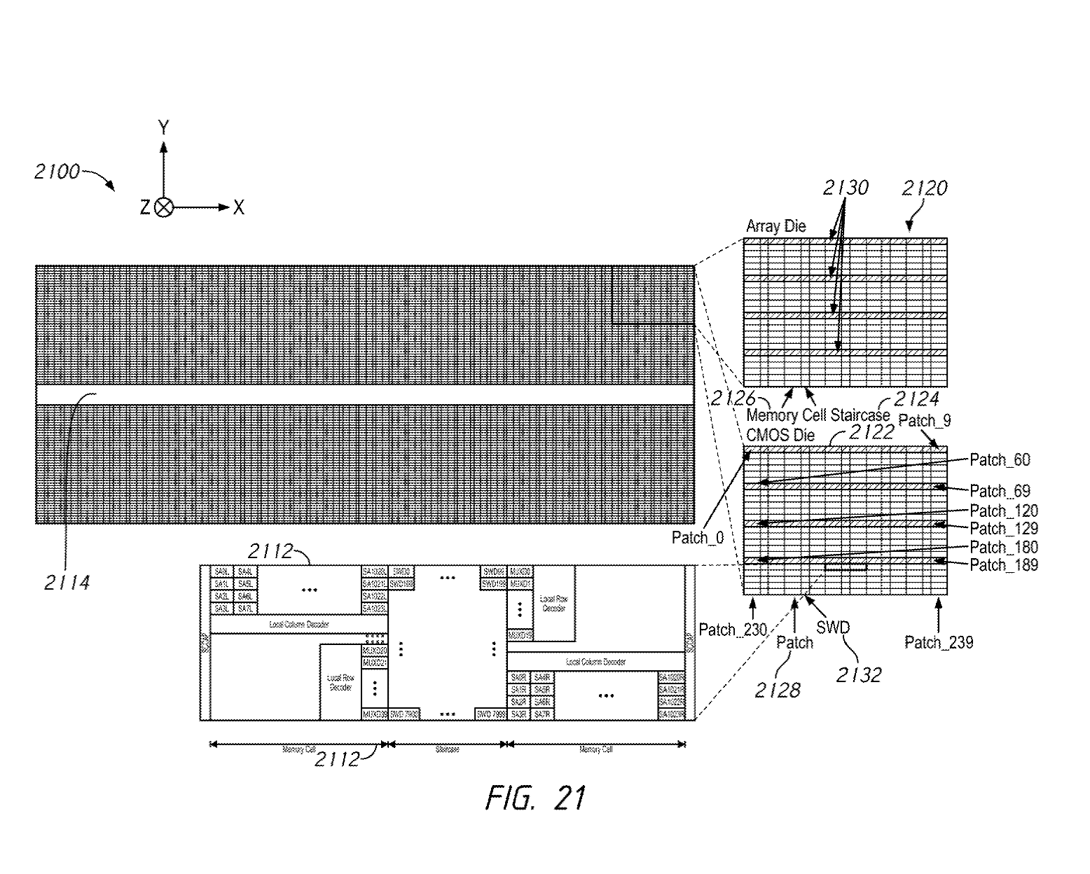

FIG. 21 is a block diagram of a 3D memory device layout according to some example embodiments of the present disclosure. The 3D memory device 2100 may, in some embodiments, represent a layout which implements the 3D memory device 100 of FIG. 1.

The view of FIG. 21 represents a view along an example x-y plane showing a top layer of a memory die, such as array die 216a or CMOS die 212a of FIG. 2A. In some embodiments, the CMOS die may be stacked above the array die in the x-direction. For example, the CMOS die and the array die may be bonded together. The memory device 2100 may include a number of memory quilts 2112, each of which includes one or more portions of memory array, such as memory patches, as well as circuitry which supports the operation of those patches such as sense amplifiers SAs (e.g., 174 of FIG. 1) and sub-word line drivers SWDs (e.g., 172 of FIG. 1). Section 2120 is an enlarged view of a memory bank on the array die and section 2122 is an enlarged view of the memory bank on the CMOS die of the memory device 2100. The memory quilts 2112 are tiled in the x-y plane of the 3D memory device 2100. The memory quilts 2112 may have a portion in the CMOS die 2122 and the array die 2120. Memory quilt 2112 is an enlarged view of an exemplary layout of a CMOS die portion of a memory quilt in the x-y plane. The example layout of the quilt 250b of FIG. 2B is shown to represent the quilt 2112. However, the layout of memory quilt 2112 may be any layout such as any of the layouts of FIGS. 2B-2G.

The memory quilts 2112 include sections of the memory array such as one or more memory array patches. In some embodiments, each quilt may include two patches and a staircase region or a SWD region between them. For example, on the array die portion of the memory bank 2120, each portion of the memory quilt may include two array patches 2126 with a staircase region 2124 between them and the CMOS die portion of each memory quilt may include two patches 2128 that include sense amplifiers and other components with a SWD region 2132 between them. The quilts 2112 may be tiled across the memory device 2100. For example, the CMOS die portion of the memory bank 2122 is depicted with 240 memory patches 2128. The topmost row of patches in the y-direction on the CMOS die portion of the memory bank 2122, shown as part of crosshatched region 2130, includes ten patches, for example, Patch_0 through Patch_9. The array patches 2126 of the array die portion of the memory bank 2120 may be similarly arranged. In some embodiments, the memory bank 2120/2122 may have more or few patches and/or other arrangements of patches.

On the array die portion of the memory bank 2120, the array patches 2126 may each include a 3D array of memory cells at the intersection of word lines and local digit lines, with the local digit lines coupled together by global digit lines. In some embodiments, a word line may be shared by multiple array patches 2126. For example, each word line may extend across two array patches 2126 in the x-direction by crossing the staircase region 2124. When viewed in the x-y plane, the word lines and the global digit lines are arranged in grid with the word lines extending in the x-direction and the global digit lines extending in the y-direction. The word lines may be stacked in the z-direction such that each word line is in a tier of the stack. For example, the tier represents the position in the z-direction of the word line in the stack. The stacks of word lines extending in the z-direction may be arranged in rows of stacks that are side-by-side in the y-direction, thus each tier may include the memory cells in an x-y plane at each position of the stack in the z-direction.

The memory device 2100 also includes a peripheral region 2114 that does not have memory quilts 2112 tiled across it. There may be a peripheral region 2114 on each die, such as the array die and the CMOS die. The peripheral regions 2114 may include other circuits which are used in the operation of the memory such as the command circuit 114 of FIG. 1, the input and output circuits 116-122 of FIG. 1, and other components.

During an example refresh operation, one or more word lines in a memory bank 2120/2122 are refreshed simultaneously. For example, the 3D memory device (e.g., 2100 of FIG. 21) may provide one or more refresh signals directed to one or more memory banks and perform one or more refresh operations in each of those banks responsive to the refresh signal REF. In some embodiments, the one or more refresh signals may be provided responsive to a self-refresh command SREF issued. For example, the self-refresh command SREF may be issued when the memory device enters a self-refresh mode. In some embodiments, the 3D memory device 2100 may refresh one word line from each of a number of quilts 2112 in each of one or more rows of quilts in a memory bank 2120/2122. In such embodiments, the number of word lines refreshed in each bank during the refresh operation is equal to the number of quilts in a row multiplied by the number of rows being refreshed. For example, the memory device 2100 may refresh one word line from each of five quilts 2112, or ten patches, in four rows of the memory bank 2120/2122. In the embodiment of FIG. 21, patches 0-9, 60-69, 120-129, and 180-189 are shown as crosshatched regions 2130 to show that a word line from each quilt containing those patches may be refreshed at the same time. The word lines being refreshed at the same time may all be at the same depth in the z-direction, or in a same tier, of the 3D memory arrays in the patches. The memory device 2100 may refresh word lines from a second set of patches in another row of the memory bank 2120/2122 after refreshing all word lines in the previous row of patches and so on until all word lines are refreshed.

In some embodiments, the word lines in each memory quilt may have a portion of the row address in common with word lines in other memory quilts. For example, a portion of the row address may be a local row address and different quilts may use the same local row addresses, and another portion of the row address may be a global row address which specifies the memory quilt 2112. For example, two or more word lines associated with a same local row address value, but with different global row address values, meaning that the rows are in multiple different memory quilts 2112, may be refreshed at the same time. For example, the global row address (or a portion thereof) may be masked in the refresh address, such that the non-masked portion (e.g., the local address) matches multiple quilts. However, during an activation operation, a single global row address is indicated such that only the word line associated with the local row address in the memory quilt associated with the single global address will be activated. By repeating local row addresses across the memory quilts 2112, the memory quilts may be manufactured with fewer row address signal lines, thus making 3D memory device layouts more efficient. In an example refresh operation, the refresh control circuit may indicate that refresh operation be performed on word lines with a local row address. For example, the refresh control circuit may refresh the word line with the specified local row address in multiple memory quilts 2112 at the same time.

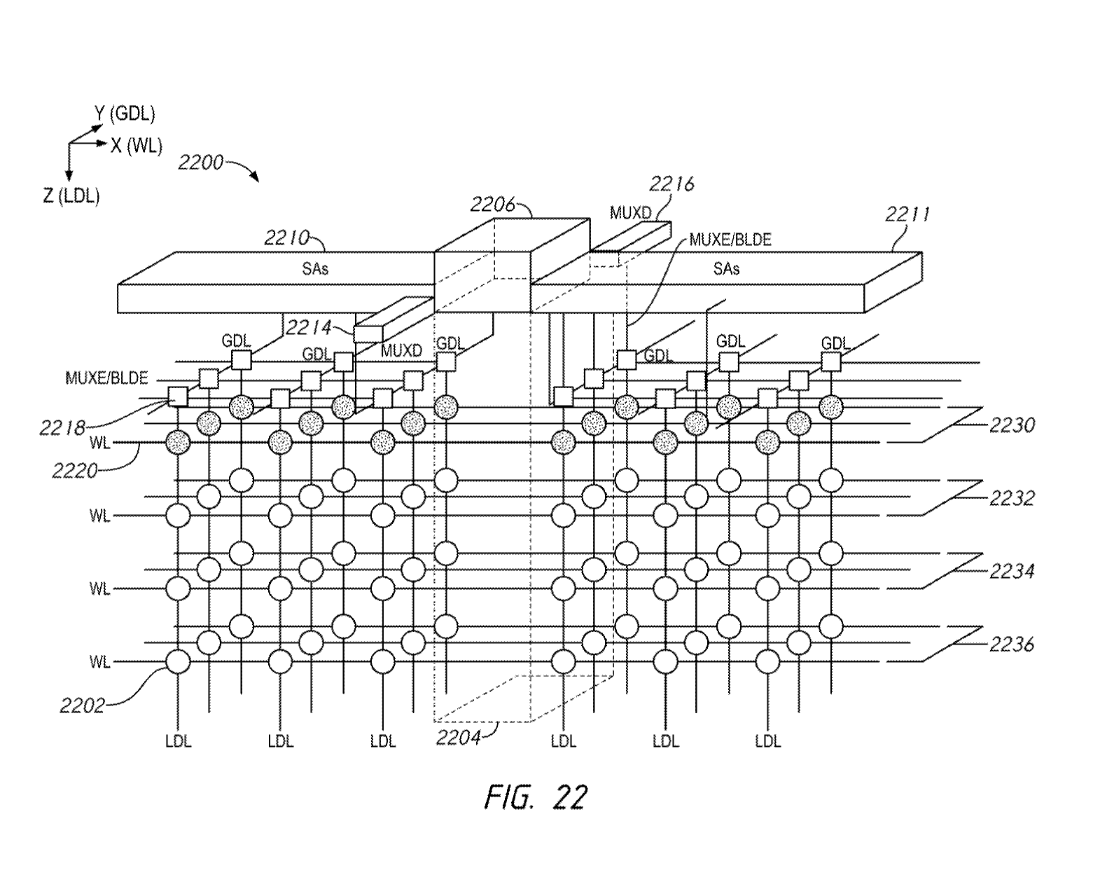

FIG. 22 is a perspective block diagram of a portion of a 3D memory device according to some embodiments of the present disclosure. The 3D memory device 2200 represents a simplified view of an example portion of a memory quilt. For example, the 3D memory device 2200 may represent a portion of the 3D device 180 of FIG. 1, a representation of a portion of the quilt 202a of FIG. 2A, and/or a representation of a portion of quilt 2112 of FIG. 21. The perspective of FIG. 22 shows an example set of memory cells 2202 (e.g., 302 of FIG. 3) and their respective word lines, local digit lines and paired global digit lines. The view of FIG. 22 may be a simplified representational view which shows a relatively small number of word lines, global digit lines, local digit lines etc. Similar to FIG. 21, FIG. 22 uses the layout of FIG. 2B as an example, however any of the example layouts of FIGS. 2B-2G may be used in embodiments of the present disclosure.

The 3D memory device 2200 shows memory arrays with memory cells 2202. Each memory cell 2202 is positioned at the intersection of a word line WL and a local digit line LDL. Each LDL is associated with one or the other of a pair of GDLs. The paired global digit lines are both coupled to a respective sense amplifier in the sense amplifier regions 2210/2211 (e.g., 222/224-226/228 of FIGS. 2 and/or 310/311 of FIG. 3). The word lines are each coupled to a respective SWD in a SWD region 2206 (e.g., 272 of FIG. 2, 306 of FIG. 3, and/or 2132 of FIG. 21). The word lines are coupled via a staircase region 2204 (e.g., 182 of FIG. 1, 282/284/286 of FIG. 2, 304 of FIG. 3, and/or 2124 of FIG. 21) to the SWD region 2206. Also shown are multiplexer driver regions 2214 and 2216 (e.g., 242/244 of FIGS. 2 and/or 314/316 of FIG. 3) which couple to multiplexer circuits 2218 (e.g., 184 of FIG. 1 and/or 318 of FIG. 3).

In some embodiments the sense amplifier regions 2210/2211, multiplexer drivers 2214/2216, and SWD region 2206 may be on a different die than the die that includes the WL, LDL, GDL, memory cells 2202, and multiplexer circuits 2218. The view of FIG. 22 shows an example embodiment where the sense amplifier regions 2210/2211 are positioned above the memory array patches. In particular, the view of FIG. 22 may represent the patches 252 and 254 of FIGS. 2, as well as their associated staircase region 282, sense amplifier regions 222 and 224, SWD regions 272, and multiplexer driver regions 242 and 246. The local column and row decoders are omitted from the view of FIG. 22.

The staircase region 2204 is a 3D region, which may be generally have the form of a rectangular prism. The staircase region 2204 is positioned underneath the SWD region 2206 in the z-direction. In some embodiments, the staircase region 2204 may have the same x-y dimensions as the SWD region 2206. The staircase region 2204 is positioned between two sections of the word lines WL which intersect memory cells 2202. However, the WLs may not intersect any memory cells while they pass through the staircase region 2204. Vertical connection elements which extend in the z-direction (not shown in FIG. 22) couple each word line to a respective SWD in the SWD region 2206. A middle of the word lines WL may be positioned in the staircase region 2204.

The word lines WL are arranged in a grid when considered in the y-z plane. Similarly, the LDLs may be arranged in a grid when considered in the x-y plane. The GDLs are generally arranged in a plane side-by-side with each other.

The LDLs are selectively coupled to one of the GDLs of the associated pair of GDLs through a multiplexer circuit 2218. Some of the LDLs along the length of the GDL pair are coupled to one of the GDLs in the pair and some of the LDLs are coupled to the other. For example, each GDL in the pair of GDLs may be coupled to roughly half of the LDLs along the length of the GDL pair. The multiplexers are coupled to multiplexer drivers in a multiplexer driver region 2214 or 2216. A line of multiplexer drivers may be coupled in common by a multiplexer driver line along the x direction to a multiplexer driver in the multiplexer driver region 2214/2216. The multiplexer driver provides a multiplexer enable signal MUXE. When MUXE is active, all the multiplexers 2218 which are coupled in common to that signal line will couple their respective LDL to the associated GDL.

In some embodiments, the multiplexer drivers 2214/2216 may also provide a bleed enable signal BLDE. When the bleed enable signal BLDE is active, the multiplexer couples the respective LDL to a ground voltage. In an example operation, the multiplexer driver associated with the row address provides an active MUXE signal and an inactive BLDE signal while the multiplexer drivers which are not associated with the row address provide an active BLDE signal and an inactive MUXE signal. Accordingly, during the operation, multiplexers which receive an active MUXE couple their LDLs to the GDL, while the other multiplexers along each GDL isolate their LDLs from the GDL and instead couple them to a ground voltage so they do not float.

In FIG. 22, memory cells 2202, shown as shaded with speckles, are located on a same tier 2230, which represents a position or depth in the z-direction of the memory cells in a 3D array. Each position in the stack of word lines extending in the z-direction may be a tier, for example tiers 2230-2236. Each tier 2230-2236 may exist as an x-y plane at a depth in the z-direction. Thus, each tier 2230-2236 may contain the word lines at that depth in the z-direction or stack position. During an example refresh operation, one word line is refreshed, such as the front word line 2220 in the y-direction, shown bolded. Multiple word lines at the same depth, or of the same tier 2230-2236, in multiple memory quilts (not shown in FIG. 22) may be refreshed at the same time. For example, a word line at the same depth in a memory quilt adjacent to memory quilt 2200 (not shown in FIG. 22) may be refreshed at the same time as word line 2220 during a memory operation. Subsequently, the 3D memory device may refresh a next word line in the stack of word lines extending in the z-direction, and in a next tier such as tier 2232, responsive to the refresh command. For example, the memory device may provide a first and second internal refresh signals responsive to a refresh command received from, for example, a memory controller. Responsive to the first internal refresh signal, the memory device may refresh memory cells 2202 of a first word line of a tier, such as word line 2220 in tier 2230, and a second word line of a second tier, such as tier 2232, below word line 2220 in the z-direction. The memory device may repeat this process responsive to refresh commands until all of the word lines are refreshed. In some embodiments, other refresh schemes may be implemented to move through all of the word lines such as refreshing the top-most word line in all of the stacks in the y-direction before moving to a next word line in the stack in the z-direction.

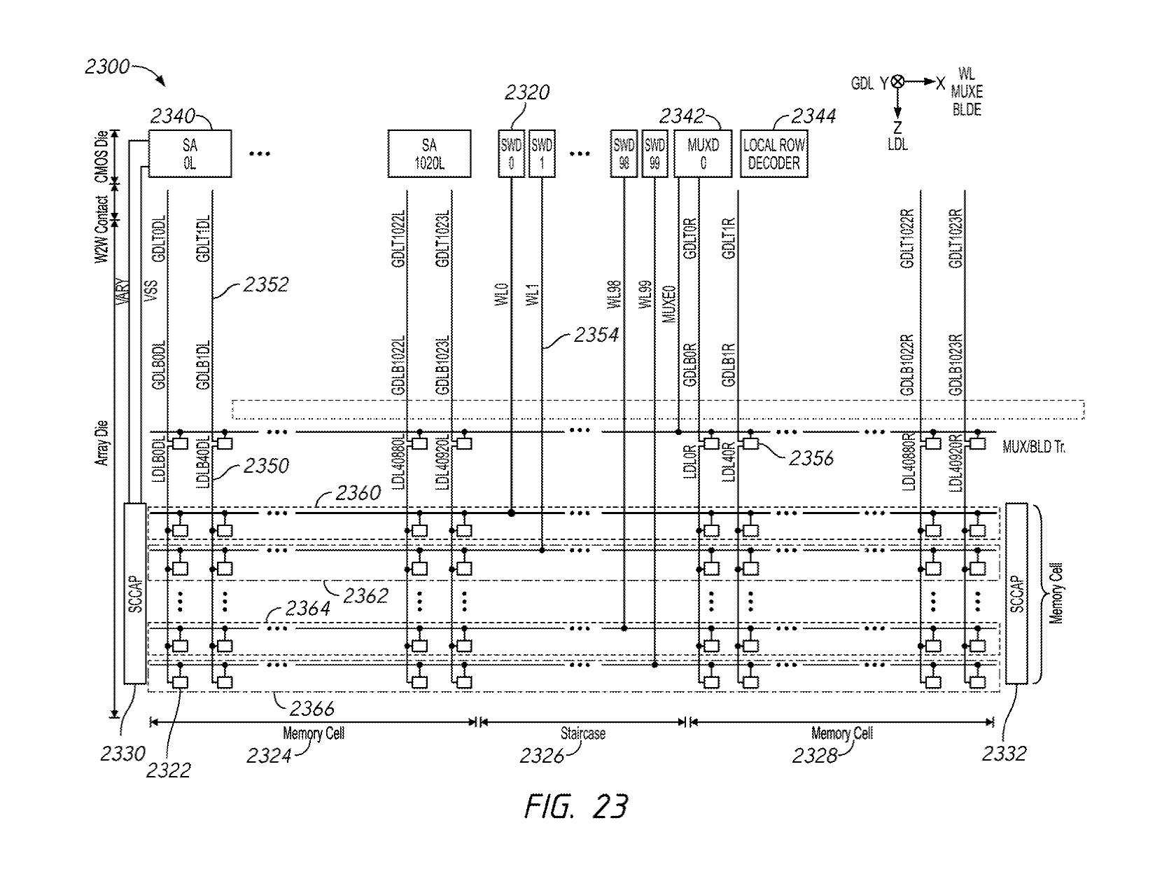

FIG. 23 is a cross sectional schematic view of a 3D memory device according to some embodiments of the present disclosure. The cross section 2300 of FIG. 23 is generally similar to the cross section 1800 of FIG. 18 thus the discussion of cross section 2300 focuses on the differences. The cross section 2300 may be an implementation of the 3D memory device 2100 of FIG. 21 and/or 2200 of FIG. 22. Similar to FIGS. 21 and 22, FIG. 23 uses the layout of FIG. 2B as an example, however any of the example layouts of FIGS. 2B-2G may be used in embodiments of the present disclosure.

The cross section 2300 represents a view along an example x-z plane, showing word lines running horizontally along the x-direction, local digit lines 2350 running vertically along a z-direction, and global digit line pairs 2352 running into and out of the plane of the page along a y-direction. Pairs of vertical conductive elements 2354 (e.g., 1814 of FIG. 18) are shown which run “behind” the plane of the cross section 2300. The vertical conductive elements 2354 are coupled to the sense amplifiers 2340 (e.g., 1812 of FIG. 18) which are positioned over the memory cells 2322. The sense amplifiers 2340 shown are in the sense amplifier portions which are positioned over a first memory patch 2324 (e.g., sense amplifier regions 222b of FIG. 2B, 310 of FIG. 3, and/or 1810 of FIG. 18). Each sense amplifier 2340 represents a “front” sense amplifier in a column of sense amplifiers that extends out of the plane of the page.

The cross section 2300 shows the first memory array region of a memory patch 2324, a staircase region 2326 (e.g., 182 of FIG. 1, 282/284/286 of FIG. 2, 304 of FIG. 3, 2124 of FIG. 21, and/or 2204 of FIG. 22), and a second array region of a second memory patch 2328. The cross section 2300 shows a row a SWDs 2320 (e.g., 172 of FIG. 1 and/or 272/276 of FIGS. 2) associated with the WLs running through the patches 2324 and 2328, a multiplexer driver 2342 (e.g., 176 of FIG. 1, 244 of FIGS. 2, and/or 316 of FIG. 3) and local row decoder 2344 (e.g., 162 of FIG. 1 and/or 264 of FIGS. 2).

The cross section 2300 shows a “stack” of word lines. In this case the word lines WL0 to WL99. A number of LDLs 2350 extend vertically in the z-direction and memory cells 2322 are coupled at the intersection of the LDLs and the WLs. The view of FIG. 23 includes digit line multiplexers 2356 (e.g., 184 of FIG. 1 and/or 318 of FIG. 3) and example multiplexer drivers 2342 (e.g., 1843 of FIG. 18). The multiplexers 2356 selectively couple a set of LDLs to one of the GDLs of the respective GDLs 2352.

In an example refresh operation, a word line 0 (WL0) (shown bolded in FIG. 23) shared by adjacent memory patches 2324 and 2328 may be refreshed responsive to a refresh signal. In some embodiments, the refresh signal may be an internal signal provided responsive to a refresh command. The refresh command may be an external command issued by a memory controller. In some embodiments, the refresh signal may be provided responsive to the memory device entering a self-refresh mode. The WL0 may be in a tier of the “stack” of word lines shown in FIG. 23 as tier 2360. Each word line in an xz cross section of the memory patch may be in a different tier. For example, in FIG. 23 each tier is shown as a dashed box including a word line. WL0 is in tier 2360, WL1 is in tier 2362, WL98 is in tier 2364, and WL99 is in tier 2366. Each tier includes a number of word lines (not shown in the cross section of FIG. 23) which are generally aligned with each other in a same xy plane, but the tiers are at different z coordinates.

The tier may represent the depth of the WL0 in the z-direction. One or more word lines at the same tier 2360 as WL0 in one or more other memory patches (not shown in FIG. 23) may be refreshed at the same time. For example, memory quilt 2300 may be an implementation of memory quilt 2112 of FIG. 21 and WL0 in each of the patches shown as crosshatched regions 2130 in memory bank 2120/2122 of FIG. 21 may be refreshed simultaneously. As shown in FIG. 21, WL0 in each of the patches in multiple rows of one or more memory banks may be refreshed at the same time. WL0 in the rows containing Patch_0 through Patch_9, Patch 60 through Patch_69, Patch_120 through Patch 129, and Patch_180 through 189 of FIG. 21 may be refreshed at the same time.

During the example refresh operation, SWD0 coupled to WL0 may be activated and WL0 may be refreshed. Similarly, in one or more other memory quilts (e.g., memory quilt 2112 of FIG. 21), SWD0 may be activated during the refresh operation and WL0 of the one or more other memory quilts may also be refreshed. WL0 in each memory quilt is at the same depth of the 3D memory arrays in the z-direction and thus, on the same tier. In some embodiments, a second refresh signal may be provided responsive the same refresh command. The second refresh signal may cause a second word line to be refreshed. For example, responsive to the second refresh signal, SWD1 may be activated and WL1 may be refreshed. SWD1 may be activated and WL1 may be refreshed in one or more memory quilts at the same time. The SWDs may be activated, and the associated word lines may be refreshed sequentially responsive to refresh signals until all word lines have been refreshed. The refresh operations may start over with SWD0 and WL0 after all the word lines have been refreshed.

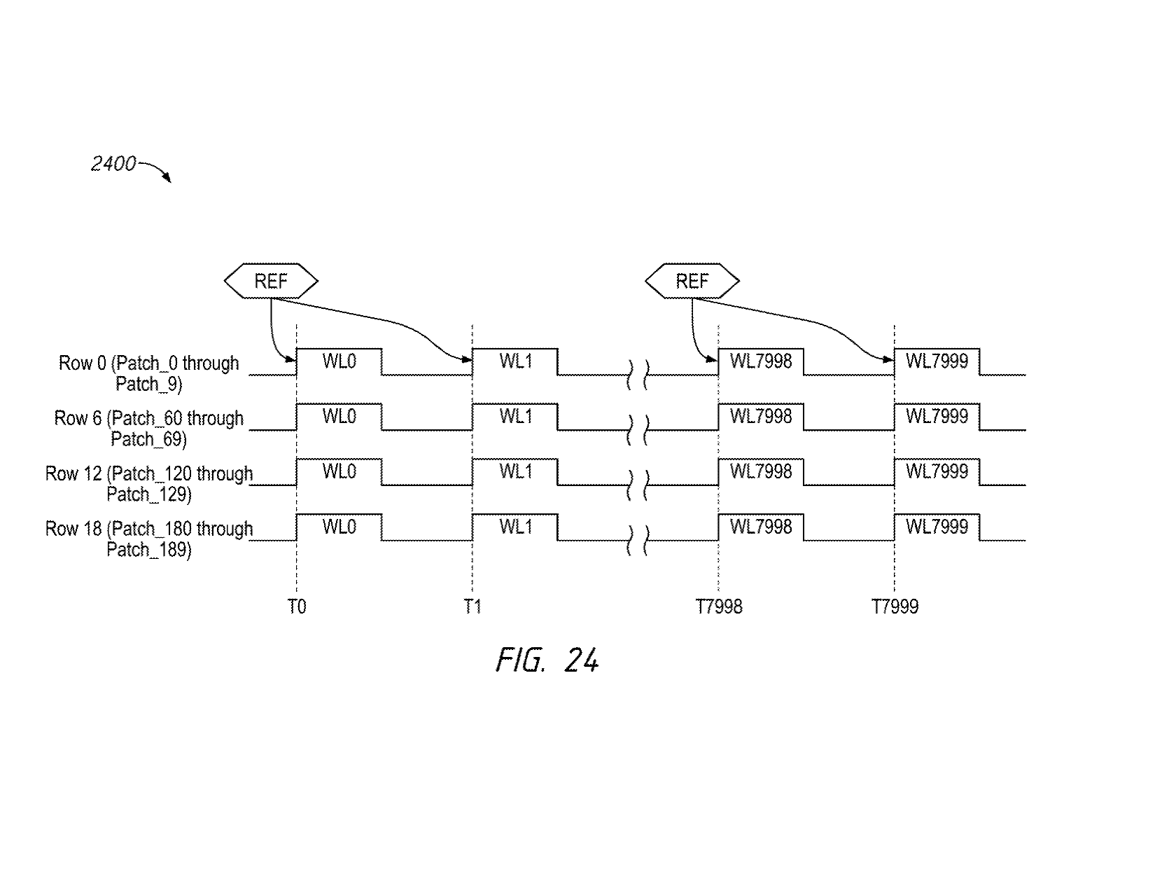

FIG. 24 is a timing diagram for example refresh operations according to some embodiments of the present disclosure. The example refresh operations shown in timing chart 2400 may be implemented on a 3D memory device, such as 3D memory device 180 of FIG. 1 and/or 200 of FIG. 2. Similar to FIGS. 21-23, FIG. 24 is described using the layout of FIG. 2B as an example, however any of the example layouts of FIGS. 2B-2G may be used in embodiments of the present disclosure.

Each of the lines of the timing chart 2400 has an x-axis which represents time. Each line represents refresh operations for word lines located in a row of patches. In the example of FIG. 24, the lines represent refresh operations for word lines in Row 0, Row 6, Row 12, and Row 18 of a memory bank. Row 0, Row 6, Row 12, and Row 18 may correspond to the rows of memory patches in the crosshatched region 2130 of the memory bank 2120/2122 of FIG. 21. For example, Row 0 may represent refresh operations on word lines located in the patches along the row of patches beginning with Patch 0 and ending with Patch_9. The row of patches beginning with Patch 0 may be the topmost row of patches of the memory banks, such as the memory bank 2120/2122 of FIG. 21 (shown as part of the crosshatched region 2130 in FIG. 21). Likewise, Row 6 may represent refresh operations on word lines located in the patches along the row of patches beginning with Patch 60 and ending with Patch_69, Row 12 may represent Patch_120 through 129, and Row 18 may represent Patch_180 through Patch_189. Word lines of more than one row of patches may be refreshed at a time, such as in Row 0, Row 6, Row 12, and Row 18 simultaneously.

Before an initial time T0, a 3D memory device including a plurality of memory banks each including a plurality of memory quilts may receive a refresh command REF. The refresh command REF may be an external command issued, for example, by a memory controller. While the refresh command REF is shown in FIG. 24, the refresh signal may be provided responsive to the memory device entering a self-refresh mode. When the 3D memory device enters the self-refresh mode, a self-refresh command SREF may be issued instead of refresh command REF. A refresh signal may be provided responsive to the self-refresh command SREF, and the refresh operation performed responsive to the self-refresh command SREF would be the same as the refresh operation responsive to the refresh command REF. The 3D memory device may enter the self-refresh mode responsive to a mode register setting.

At time T0, responsive to the refresh command REF, the memory device may provide a first refresh signal. The first refresh signal may cause the 3D memory device to perform a refresh operation on a first word line WL0 of each patch in rows of patches Row 0, Row 6, Row 12, and Row 18. For example, the word line WL0 of each of the patches in the crosshatched regions 2130 of the memory bank 2120/2122 of FIG. 21. The first word line refreshed in each memory patch may be at a same tier in each patch. During the refresh operation, a first SWD may be activated in each memory quilt and the first SWD may be coupled to the first word line which is at a same depth in the z-direction in each patch.

At time T1, responsive to the refresh command REF, the 3D memory device may provide a second refresh signal. The second refresh signal may cause the 3D memory device to perform a refresh operation on a second word line WL1 of each patch in the rows of patches Row 0, Row 6, Row 12, and Row 18. For example, the word line WL1 of each of the patches in the crosshatched regions of the memory bank 2120/2122 of FIG. 21. The word line WL1 refreshed in each memory patch may be at a same tier. During the refresh operation, a second SWD may be activated in each memory quilt and the second SWD may be coupled to the second word line which may be below the first word line in the z-direction. The second word line WL1 may be at the same tier in each patch in the rows of patches Row 0, Row 6, Row 12, and Row 18 or in other words at the same depth in the z-direction.

At time prior to T998, a second refresh command REF may be issued. Time T998 may be any amount of time, such as a longer time after T1 than the amount of time between T0 and T1, as shown by the breaks in the lines. During the time between T1 and T7998, one or more refresh commands may be issued. The frequency at which the word lines are refreshed, or the maximum time any given word line will go between refreshes, may be determined based on a specification.

At time T7998, which is many refresh signals REF after T1, responsive to another refresh command REF, the memory device may provide another refresh signal. The another refresh signal may cause the 3D memory device to perform a refresh operation on another word line WL7998 of each patch in the rows of patches Row 0, Row 6, Row 12, and Row 18. The another word line WL7998 refreshed in each memory patch may be in another tier of the patch. For example, another SWD may be activated in each memory quilt and the another SWD may be coupled to the another word line WL998 at a another depth in the array in the z-direction.

At time T7999, responsive to the another refresh command REF, the memory device may provide another internal refresh signal. The another internal refresh signal may cause the 3D memory device to perform a refresh operation on another word line WL7999 of each patch in the rows of patches Row 0, Row 6, Row 12, and Row 18. The word line WL7999 refreshed in each memory patch may be at another tier of the patch. For example, another SWD may be activated in each memory quilt and the another SWD may be coupled to the word line WL7999 which may be below the word line WL7998 in the z-direction. The word line WL7999 may be at the same tier in each patch in the rows of patches Row 0, Row 6, Row 12, and Row 18 or in other words at the same depth in the z-direction.

In the example of FIG. 24, each quilt contains 8000 word lines arranged in a grid in the y-z plane with 80 word lines in the y-direction and 100 word lines in the z-direction. In other embodiments, there may be more or fewer word lines. Each memory bank (e.g., 2120 of FIG. 21) may contain 240 patches or 120 quilts. In the example refresh operation of FIG. 24, two word lines from each patch in four rows of the memory bank are refreshed responsive to each refresh command REF. In other words, two internal refresh signals are provided responsive to each refresh command REF. In some embodiments, more or fewer internal refresh signals may be provided. Responsive to each refresh signal, one word line is refreshed in each patch and all of the word lines refreshed at a time are at a same tier or depth in the z-direction. In some embodiments, more than one word line may be refreshed in each patch responsive to the internal refresh signal. In the example of FIG. 24, time T7999 may be a total amount of time to refresh the entire memory array. Some standards may call this time a refresh period.

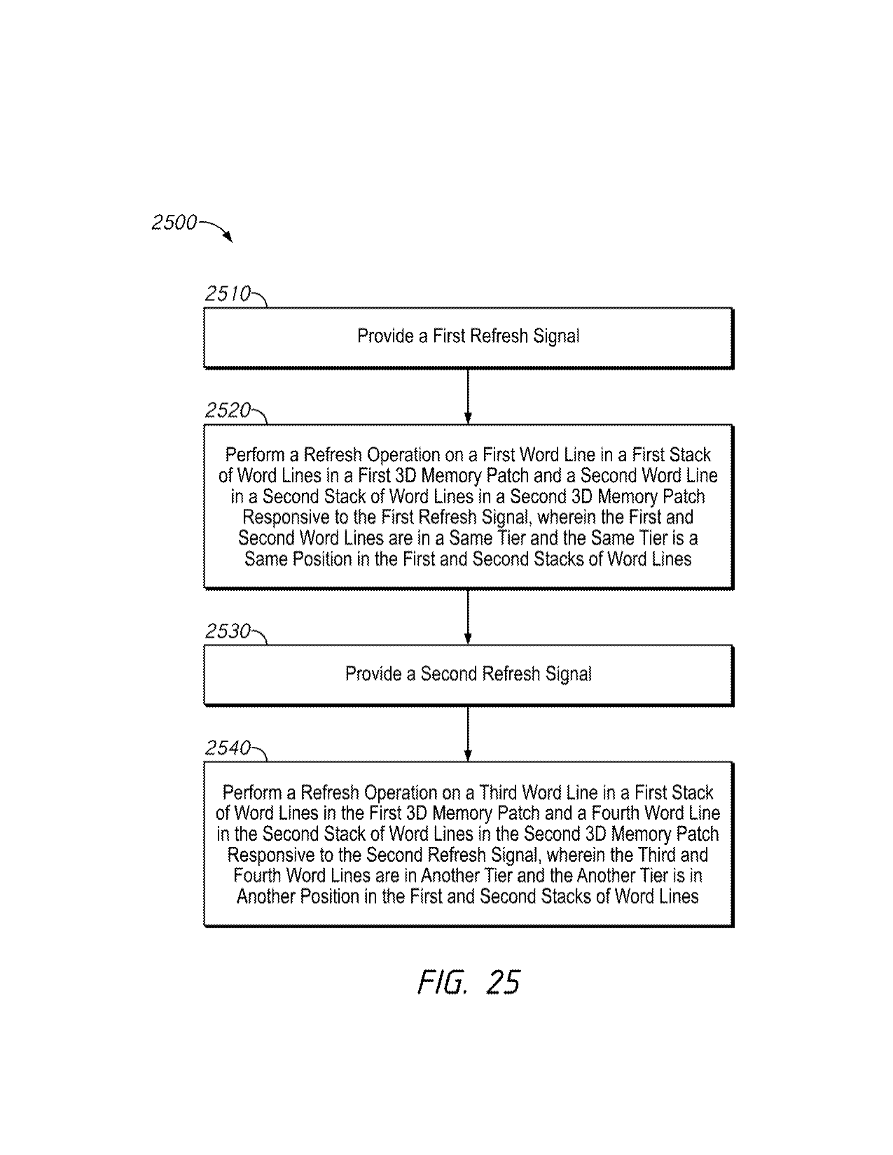

FIG. 25 is a flow chart of a refresh operation according to some embodiments of the present disclosure. The method 2500 may, in some embodiments, be implemented by a 3D memory device such as 3D memory device 2100 in FIG. 21. Similar to FIG. 24, the method of FIG. 25 is described using the layout of FIG. 2B as an example, however any of the example layouts of FIGS. 2B-2G may be used in embodiments of the present disclosure.

The method 2500 may begin at block 2510, which describes performing a first refresh operation on a first word line which is in a first stack of word lines in a first 3D memory patch and a second word line which is in a second stack of word lines in a second 3D memory patch responsive to a first refresh signal, wherein the first and second word lines are in a same tier and the same tier is a same position in the first and second stacks of word lines. In some embodiments, the first refresh signal may be provided responsive to a refresh command. For example, the refresh command may be an external command, such as issued by a memory controller. In some embodiments, the first refresh signal may be provided responsive to the memory device entering a refresh mode, such as a self-refresh mode. For example, the 3D memory device may enter the self-refresh mode responsive to a mode register setting.

In some embodiments, the first and second 3D memory patches (e.g., 2324/2328 of FIG. 23) may be located in rows of a memory bank (e.g., 2130 of FIG. 21). The first word line in each of the 3D memory patches may be in a same tier (e.g., tier 2230 of FIG. 22 and/or tier 2360 of FIG. 23). For example, each first word line may be at the same depth or position in the z-direction in the stack of word lines. In some embodiments, the first refresh operation may include activating a first SWD coupled to the first word line and activating a second SWD coupled to the second word line. The method 2500 may proceed to block 2520.

Block 2520 describes performing a second refresh operation on a third word line which is in the first stack of word lines in the first 3D memory patch and a fourth word line which is in the second stack of word lines in the second 3D memory patch responsive to a second refresh signal, wherein the third and fourth word lines are in another tier and the another tier is in another position in the first and second stacks of word lines. In some embodiments, the second refresh signal may be provided responsive to the refresh command. For example, the refresh command may be an external command, such as issued by a memory controller. In some embodiments, the second refresh signal may be provided responsive to the memory device entering a refresh mode, such as a self-refresh mode.

In some embodiments, the first and second 3D memory patches (e.g., 2324/2328 of FIG. 23) may be located in rows of a memory bank (e.g., 2130 of FIG. 21). The second word line in each of the array patches may each be at a same tier (e.g., tier 2232 of FIG. 22 and/or tier 2362 of FIG. 23). For example, each second word line may be at the same depth, or position, and below the first word line in the z-direction in the stack of word lines. In some embodiments, the second refresh operation may include activating a third SWD coupled to the third word line and activating a fourth SWD coupled to the fourth word line.

In some embodiments, the method 2500 may further include providing a refresh signals and performing a refresh operations on each word line in the stack of word lines on the plurality of 3D memory array patches until all word lines are refreshed. The method 2500 may then start back over with the first word line.

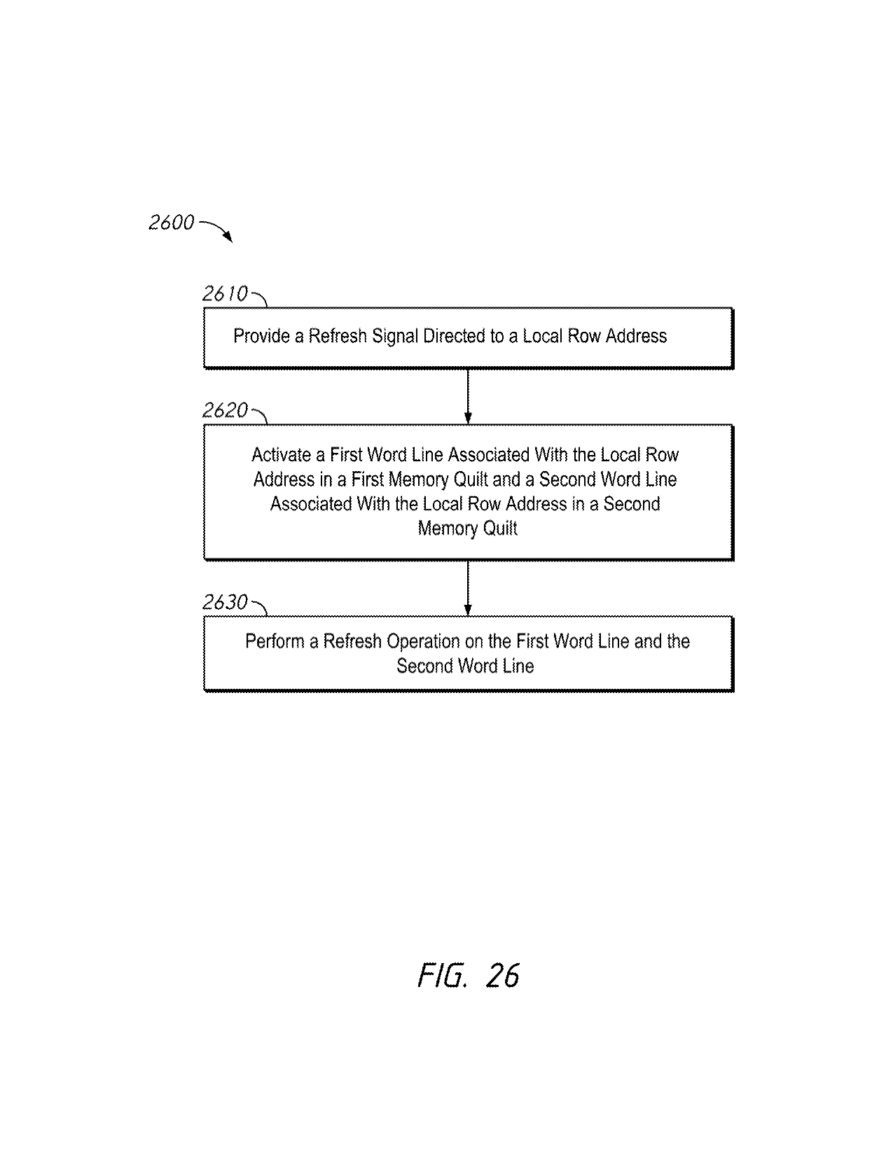

FIG. 26 is a flow chart of a refresh operation according to some embodiments of the present disclosure. The method 2600 may, in some embodiments, be implemented by a 3D memory device such as 3D memory device 2100 in FIG. 21. Similar to FIG. 25, the method of FIG. 26 is described using the layout of FIG. 2B as an example, however any of the example layouts of FIGS. 2B-2G may be used in embodiments of the present disclosure.

The method 2600 may begin at block 2610, which describes providing a refresh signal directed to a local row address. In some embodiments, the refresh signal may be provided by the 3D memory device (e.g., 2100 of FIG. 21) and directed to one or more memory banks to cause the one or more memory banks to perform one or more refresh operations responsive to the refresh signal REF. In some embodiments, the one or more refresh signals may be provided responsive to a self-refresh command SREF issued. The method 2600 may proceed to block 2620. The method 2600 may proceed to block 2620.

Block 2620 describes activating a first word line associated with the local row address in a first memory quilt and a second word line associated with the local row address in a second memory quilt. In some embodiments, the word lines in each memory quilt may have a portion of the row address in common with word lines in other memory quilts. For example, a portion of the row address may be a local row address and different quilts may use the same local row addresses, and another portion of the row address may be a global row address which specifies the memory quilt 2112. For example, two or more word lines associated with a same local row address value, but with different global row address values, meaning that the rows are in multiple different memory quilts 2112, may be refreshed at the same time. For example, the global row address (or a portion thereof) may be masked in the refresh address, such that the non-masked portion (e.g., the local address) matches multiple quilts. The method 2600 may proceed to block 2630.

Block 2630 describes performing a refresh operation on the first word line and the second word line. Responsive to the refresh signal, the 3D memory device (e.g., 2100 of FIG. 21) may perform the refresh operation on the first word line associated with the local address and located in the first memory quilt (e.g., 2112 of FIG. 21) and the second word line associated with the local address and located in the second memory quilt (e.g., 2112 of FIG. 21). The first and second word lines associated with the local address may be refreshed simultaneously. In some embodiments, word lines associated with the local address across more than two memory quilts of a 3D memory device may be refreshed at the same time.

Of course, it is to be appreciated that any one of the examples, embodiments or processes described herein may be combined with one or more other examples, embodiments and/or processes or be separated and/or performed amongst separate devices or device portions in accordance with the present systems, devices and methods.

Finally, the above-discussion is intended to be merely illustrative of the present system and should not be construed as limiting the appended claims to any particular embodiment or group of embodiments. Thus, while the present system has been described in particular detail with reference to exemplary embodiments, it should also be appreciated that numerous modifications and alternative embodiments may be devised by those having ordinary skill in the art without departing from the broader and intended spirit and scope of the present system as set forth in the claims that follow. Accordingly, the specification and drawings are to be regarded in an illustrative manner and are not intended to limit the scope of the appended claims.

What is claimed is:

1. An apparatus comprising:
a first word line which is in a first stack of word lines in a first 3D memory array patch;
a second word line which is in a second stack of word lines in a second 3D memory array patch, wherein the first word line and the second word line are in a same tier and the same tier is a same position in the first and second stacks of word lines;
a third word line which is in the first stack of word lines in the first 3D memory array patch;
a fourth word line which is in the second stack of word lines in the second 3D memory array patch, wherein the third word line and the fourth word lines are in another tier and the another tier is another position in the first and second stacks of word lines; and
a refresh control circuit configured to perform a first refresh operation on the first and second word lines simultaneously and a second refresh operation on the third and fourth word lines simultaneously.

2. The apparatus of claim 1, further comprising:
a first sub-word line driver coupled to the first word line;
a second sub-word line driver coupled to the second word line;
a third sub-word line driver coupled to the third word line; and
a fourth sub-word line driver coupled to the fourth word line, wherein the first and second sub-word line drivers are activated responsive to the first refresh operation and the third and fourth sub-word line drivers are activated responsive to the second refresh operation.

3. The apparatus of claim 1, further comprising:
a first die, wherein the first die includes the first and second 3D memory array patches; and
a second die, wherein the second die includes the refresh control circuit and wherein the first die and the second die are stacked.

4. The apparatus of claim 1, wherein the third word line is below the first word line in the first stack of word lines and the fourth word line is below the second word line in the second stack of word lines.

5. The apparatus of claim 1, wherein the first and second refresh operations are performed responsive to a refresh command.

6. The apparatus of claim 1, wherein the first and second refresh operations are performed responsive to a self-refresh mode.

7. An apparatus comprising:
a plurality of pairs of 3D memory array patches, each pair comprising a plurality of memory cells arranged at the intersection of a plurality of word lines and a plurality of local digit lines, wherein sets of the plurality of local digit lines are coupled to one of a plurality of global digit lines and wherein the plurality of word lines extend in a first direction, the plurality of local digit lines extend in a second direction orthogonal to the first direction, and the plurality of global digit lines extend in a third direction orthogonal to the first and the second direction and wherein the plurality of word lines are organized in one or more stacks extending in the second direction, each of the plurality of word lines having a tier representing its position in the respective one of the one or more stacks in the second direction; and
a refresh control circuit configured to perform a refresh operation on a first set of word lines, wherein the first set of word lines comprises a word line of each plurality of word lines of each pair of 3D memory array patches of the plurality of pairs of 3D memory array patches and wherein each word line of the first set of word lines is in a same tier of the one or more stacks of word lines.

8. The apparatus of claim 7, wherein the refresh control circuit is further configured to perform a second refresh operation on a second set of word lines, wherein the second set of word lines comprises a second word line of each plurality of word lines of each pair of 3D memory array patches of the plurality of pairs of 3D memory array patches and each word line of the second set of word lines in in a second tier of the one or more stacks of word lines.

9. The apparatus of claim 8, wherein the second tier is below the first tier in the second direction.

10. The apparatus of claim 7, further comprising:
a first die, wherein the first die includes the plurality of pairs of 3D memory array patches; and
a second die, wherein the second die includes the refresh control circuit and wherein the first die and the second die are stacked.

11. The apparatus of claim 10, further comprising a plurality of sub-word line drivers and a plurality of sense amplifiers, wherein each sub-word line driver of the plurality of sub-word line drivers and each sense amplifier of the plurality of sense amplifiers is coupled to a word line of the plurality of word lines and wherein the second die includes the plurality of sub-word line drivers and the plurality of sense amplifiers.

12. The apparatus of claim 7, wherein the refresh operation is performed responsive to a refresh command.

13. The apparatus of claim 12, wherein the refresh command is issued by a controller.

14. The apparatus of claim 7, wherein the refresh operation is performed responsive to a self-refresh mode.

15. A method comprising:
performing a first refresh operation on a first word line which is in a first stack of word lines in a first 3D memory patch and a second word line which is in a second stack of word lines in a second 3D memory patch responsive to a first refresh signal, wherein the first and second word lines are in a same tier and the same tier is a same position in the first and second stacks of word lines; and
performing a second refresh operation on a third word line which is in the first stack of word lines in the first 3D memory patch and a fourth word line which is in the second stack of word lines in the second 3D memory patch responsive to a second refresh signal, wherein the third and fourth word lines are in another tier and the another tier is in another position in the first and second stacks of word lines.

16. The method of claim 15, wherein the first refresh signal is provided responsive to receiving a refresh command.

17. The method of claim 16, wherein the refresh command is issued by a controller.

18. The method of claim 15, wherein the first refresh signal is provided responsive to entering a self-refresh mode.

19. The method of claim 15, wherein the first refresh operation further includes activating a first SWD coupled to the first word line and activating a second SWD coupled to the second word line and the second refresh operation further includes activating a third SWD coupled to the third word line and activating a fourth SWD coupled to the fourth word line.

20. The method of claim 15, wherein the first and second refresh signals are provided responsive to a same refresh command.
